SEQUENCE LISTING (89 sequences)

SEQ ID NO: 1 - DNA, 8131 bp, Artificial Sequence
  Biosynthetic Gene Cluster (BGC)
  misc_feature (1767)..(1769): n is a, g, c or t each nucleotide is a nucleotide which is part of a base triplet coding for an amino acid
  misc_feature (1773)..(1784): n is a, g, c or t each nucleotide is a nucleotide which is part of a base triplet coding for an amino acid
  misc_feature (2073)..(2075): n is a, g, c or t each nucleotide is a nucleotide which is part of a base triplet coding for an amino acid
  misc_feature (2079)..(2090): n is a, g, c or t each nucleotide is a nucleotide which is part of a base triplet coding for an amino acid
cgcgcgcgattctaaccggtataagaaataaagtggagcgccggcgattatccctaggtcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcataataccttaaatgaaaccgttaaaactcaagaagcactcaattctcttgctgcatcattcaaagagactgaactctcaattactgataaagcactaaacgaattaagcaataaacctaagatccctgagatcacggcctggnnntggnnnnnnnnnnnncaggaaatttaaagcttatcccatcaggttattttattttcctgaaaaaacaatgtccattgggataaacaatcaagttgttcagttaaaataaataattttttgaaccttaatcatagggtaatcctatgtattatccgggattaccctaaatactttcataaagtaaatataaaaatttgaataatacagataagtttttttaacctgaatggaaggtttgtattttatcattaaacacaaccaccttttaccactaatattattggattttttcatttcaagatgaaaatataatagatttcagtaactaaaaagggtaatagcctgtcccagcaggtgttactgatgcaggcaaataaaatagccctaataggatgggctttaagtaaggaacggtagccataactgcagtcctgaggatccccaccctgcaagggcccggaaccgcaaggttccgggcccttcgccttttatcaggttatttgcggccgctggacagcaagcgaaccggaattgccagctggggcgccctctggtaaggttgggaagccctgcaaagtaaactggatggctttcttgccgccaaggatctgatggcgcaggggatcaagatctgatcaagagacaggataaggaggtacagattatgaatgctattaacatctcagaatccatgaaacgtttcaatatattggtatcgatactctcagtatcaatagcaatattagcaatttatttactttctactgccttctattcagatgctcgagttgataactattatcaagataatggcaacatttaccgggtggaaaccacctttaatttgcccaatggagaaaaagtgagatcggcaaaatcgccattgccgctcatagacgagctagaaaaagataaaaggataaatcgtgcagactactttttcaaacttaacacgacgattgagttacaaggaaaaaagatcaccaaagttcccgtgtttgcagtcagtcatcaattcctgaataccctgtctccctttaagaaaatcgataatatcctgggagcaaatgagatttacatcacaaaagagttcaataatcgctatctcggcctggaaaatcccaaaggaaaatctattgttctggataatgaaacctatatcatcaaggatatcgtagaaaaacgtcatgattcgagtcttaatatgaatgctattatttcatttaagccaacacttatcaaaaactacaataaagaaatattcaattggtatgatacacatgcttatatttttataaaactctctgatgaaaaatcaatatccaatcgcgatgcgcttcttaatcatattattgcaaccaaagcccccaatcttccaggcgctccatttactgccagtgaatttattcacctatcattaaaaaaatcaaaagatattcattaccaagatggattgcctgatgaaatttctattggtttatcaaaggagataatttatagctcatatatcgcgttcgcatttatcttattttccacattaaccaactattataatttaacttctgctgattcagcagaaaaaagggatatttatcagttaaagaaagcgctaggcgcttcaactttcaatattatttgcgattcactgccggccatgttgatcaaaataatcctttccacccttatctttttgttacttatcataactatatttaatgtctctctctatataaataatatatttaccctattagaagcagacagtttaatatatctagggatattttcagttgttattgcctttatctttatttcatcaatacatttttcattctcattacgtttttatatttatcaaagaaacagaaaaatggatatgagatatgaatcaacccttgcctcttggtttaggaaatcatcattggctattcaattacttgtttccggaatatctatttatttagtcaccggattaaccaatcaatatattcaccttgttgatttacatactccttatgataatgccaagacaatatcaatagcaattaataacgataataataaaaaatcaactattgatgaattaagctatgagtttatcagaaaatacaatacttcaaaaataaccctcagtaattggcgaccatttgatatgtcaagagaaaatatcactattaattattccggccaacaaaacaggaataactatatctcttcaaatgttattactgcggatgaaaatttcactgatgtgtggagaatgaagatattagccggtggtgataaacgagtatatcagagtgataatgctgatgtagtccatgcattagtgacaaaagaatttttaaaacagaatggaattctcaaatatgatgatatttttaacaactactattggtacacaatggaggataaaaagatccaaatcagatttattcagattatagatgatttcaatttaggtgctgtagatgaaccattcaggccaatagttgttttcatcaaaaaagatcatggaaaatatgcttcgctgaatttaaacaatatgaaaaacctttctccggtacttaacgaactatcaaaacagggttttgataataatgaaattgatttaaccagtcacttatttaactcttattatgataattatttaaaagtaataaaattatcatctgccgtcgctattttctcggttctattattagtcatcgcctgctttactaccagcatgactgactttaacgcgatgaaaagggagttaagtataatggaatctattggtggttctatttacaccaatctcattcactttttatcgaaaagtgtcatcccgataatcgcatcaattttcgtggcatatatatcaggaagaactattttagtttatctgcttgatgaatatagtgtgatttacaccgattcttttgtatacagtgctatcgccctatctgctacaatgatattcaccgttattataatgatgactacctatttggtgaattatagaaaattatccgccactaaatttttgtgagattaattatggatatagaaatcagaaaaaaaggcatacataacagagagaaattactcattctcattttctttatcgcttttatttcatttgttgtatttattatctattatgtcgtttcggctaaagacgctttcgtgaatgaaaatgaagttacccagtttcaggtcagcagcaaagtagatagtgatattctgaaaaccagggcagttgtggttcctgataaaagtgtctctatctcaaccgagatagggggaatcgttaccgatataatgaaaaaaccatcacaagatgttttaaagaatgaagaaatcgtgaaattatctaattttaatttcacattgaataattcttcgatgttagcggacgtgacagataaactcaataatttaataaacataagaataaacttgcaatctgactatcgggatatcaataaccgattcttagaagccgccaaagaactaaaagaagtagaagataaattaaagcgatatcacgatcttgtacataaaaattatatttctaaagaaactatgtccgatttaaggattaaaagagattattggcacgatgtttatctattctacaaaaaattaaaaaatgataaagatcgtgatatatcaaaacagttaaaggaaatcgacgagtttgtcgaaaaacagagaaaactctctgacatcatcgaaaatggtttcgagcagctttcaataaaatcccctatcgccggtaatatcagttcattagatttaattttaggacaacggttaaaacctggcgataaaatagctattgttgatgatttatcaaacttttattttgaatcagaaatcaacgaatactatttaaacaaaataacataccattcatctgccagtttaatctataacaatggaaaaatacctttactggtgaaattaatttcatctgaagtgaataatggaacattcaaagtcagatttgaattggaagataaaaaaacaattaacttcaaacgggggcaatccgttgatgtcatgattaaacttgatgaagatcggaaaatattctccgtaccttcatctatggtgttttctattgacaataaaaactatgtgtttgtttaccatccagataaaaaaattgcccgacgtattcaagtttccaccggccaagataatggttctgatattgagatcaaaagtggtgttactgaaggacaaacgctcgttagctttggtaagaataaattagttaataatgatacagtaaggattgaataaaatgattagcatgatgaatatctgtaaatcatataaaacgaaattcattcaaacaaatgttttgaatgacataaatctcaatatagataaaggcgaatttatttctataatgggagcgtcagggtcagggaaatcaacattactgaatgtcattggcatgtttgaaacaatcgatagcggtcaattaacactgaacaatagcaatattttaacaatgaaatattctgaaaaaatatcattcagacgagaatttattggttatatttttcaatcattcaatctgttacccaatttgacggtatttgaaaacatagagttaccgttaaaatacagaggttttcctaaaaaacagcggaaaagcaaagtatttgatgcaataaatagttttggattagaaaatcgggaaaatcataaaccgatacaattatctggtggacaacagcaacgtgttgctatcgccagagcaatgatagcaaaccccaccattttattagctgatgaaccaacggggaacctggatagcgtaaatggtcagaatattttatcttcacttaaggagctgaatgaaaacggtacgacgattgttatggtaacccattccgtagaagcggcagcgttttctgacagaatattaacgatgagggatggccacttgcttgtttgagcataaataacataataaaggagttctttatggacacaataatccccataaaatatttagattcagacgaatcatcgattcttaagaaatcatctaaaattaactacaggcaattagcttgcagaattatcggtgaaatctccgccgaaaaaatattagatgatgatgaactggctttatataataaagaaatcagtatacatttcagccctgaaattattaatgctaataaattagttgtggttgtgaaagccaccaggctttgcaatttaagatgcacttattgtcactcctgggcagaaggaaaaggaaataccttaacattcttcaatttaatgcgttccattcaccgtttcttatccctaccgaatatcaagcgatttgaattcgtctggcatggcggcgaagtaacgttgttgaatgttaattactttaagaaactcatctggttacaggaacaatttaaaaaaccggatcaagttatcaccaattcggtacagacaaatgccgtcaatattcctgaagattggttagtgttcctcaaaggtattggaatgggggtaggaataagcgttgatggtattccggaaatacacgatagcaggagattagattacagaggaaggccaacatcccataaagtcgcggcaagtatgaaaaagttaagaagttatggcataccttacggtgcgcttatcgtcgtcgaccgcgatgtttatgaatcaaatatagaaaaaatgctctcttatttttacgaaatcggtttaacggatattgaatttctgaatattgtcccagataaccgatgccagccgggtgatgatcctggaggaagttatataacttaccataactatattaatttcctttctaaggttttccgtgtctggtggaatggttatcaaggcaaaatcaatattcgcttgtttgacggatttattgacagtatcaaatcgtcccaaaagaaaatgtcagattgttattgggcgggtaactgttctcaggaaataatcacattagaacctaatggtacggtatcagcatgtgataaatatgttggtgctgaagggaataattatggttcgattattgataatgatcttgggaatttactatctaaatcaaatacaaataaggatcatcttaaagaggaaatggaatcttatgaaaaaatgcatcaatgtaaatggtttcatttgtgtaatggtggatgcccacacgatcgagtgaccaacaggaagcacaatccaaattatgatggttcatgttgtggaaccggcggtttgttggagacaataaaacaaaccatcgcggcgtaaattctaactagttcctgattaattaaccaccctgcaagggcccggaaccgcaaggttccgggcccttcgccttttcgtcgacg

SEQ ID NO: 2 - DNA, 21 bp, Photorhabdus khanii
  CDS (1)..(21): Propetide core sequence - Darobactin A
tggaactggtcaaaaagcttc

SEQ ID NO: 3 - protein, 7 aa, Photorhabdus khanii
WNWSKSF

SEQ ID NO: 4 - DNA, 21 bp, Artificial Sequence
  Propetide core sequence - Darobactin B
  CDS (1)..(21): Propetide core sequence - Darobactin B
tggaactggaccaaacgattc

SEQ ID NO: 5 - protein, 7 aa, Artificial Sequence
  Synthetic Construct
WNWTKRF

SEQ ID NO: 6 - DNA, 21 bp, Artificial Sequence
  Propetide core sequence - Darobactin C
  CDS (1)..(21): Propetide core sequence - Darobactin C
tggtcatggtcaagaagcttc

SEQ ID NO: 7 - protein, 7 aa, Artificial Sequence
  Synthetic Construct
WSWSRSF

SEQ ID NO: 8 - DNA, 21 bp, Artificial Sequence
  Propetide core sequence - Darobactin D
  CDS (1)..(21): Propetide core sequence - Darobactin D
tggaactggtcaagaagcttc

SEQ ID NO: 9 - protein, 7 aa, Artificial Sequence
  Synthetic Construct
WNWSRSF

SEQ ID NO: 10 - DNA, 21 bp, Artificial Sequence
  Propetide core sequence - Darobactin E
  CDS (1)..(21): Propetide core sequence - Darobactin E
tggtcatggtcaaaaagcttc

SEQ ID NO: 11 - protein, 7 aa, Artificial Sequence
  Synthetic Construct
WSWSKSF

SEQ ID NO: 12 - DNA, 21 bp, Artificial Sequence
  Propetide core sequence - Darobactin 1
  CDS (1)..(21): Propetide core sequence - Darobactin 1
tggtcatggaccaaacgattc

SEQ ID NO: 13 - protein, 7 aa, Artificial Sequence
  Synthetic Construct
WSWTKRF

SEQ ID NO: 14 - DNA, 21 bp, Artificial Sequence
  Propetide core sequence - Darobactin 2
  CDS (1)..(21): Propetide core sequence - Darobactin 2
tggtcatggaccagaagcttc

SEQ ID NO: 15 - protein, 7 aa, Artificial Sequence
  Synthetic Construct
WSWTRSF

SEQ ID NO: 16 - DNA, 21 bp, Artificial Sequence
  Propetide core sequence - Darobactin 4
  CDS (1)..(21): Propetide core sequence - Darobactin 4
tggaactggaccaaaagcttc

SEQ ID NO: 17 - protein, 7 aa, Artificial Sequence
  Synthetic Construct
WNWTKSF

SEQ ID NO: 18 - DNA, 21 bp, Artificial Sequence
  Propetide core sequence - Darobactin 5
  CDS (1)..(21): Propetide core sequence - Darobactin 5
tggtgctggtcaaaaagcttc

SEQ ID NO: 19 - protein, 7 aa, Artificial Sequence
  Synthetic Construct
WCWSKSF

SEQ ID NO: 20 - DNA, 21 bp, Artificial Sequence
  Propetide core sequence - Darobactin 6
  CDS (1)..(21): Propetide core sequence - Darobactin 6
tggaactggtgcaaaagcttc

SEQ ID NO: 21 - protein, 7 aa, Artificial Sequence
  Synthetic Construct
WNWCKSF

SEQ ID NO: 22 - DNA, 21 bp, Artificial Sequence
  Propetide core sequence - Darobactin 7
  CDS (1)..(21): Propetide core sequence - Darobactin 7
tggtgctggtgcaaaagcttc

SEQ ID NO: 23 - protein, 7 aa, Artificial Sequence
  Synthetic Construct
WCWCKSF

SEQ ID NO: 24 - DNA, 21 bp, Artificial Sequence
  Propetide core sequence - Darobactin 8
  CDS (1)..(21): Propetide core sequence - Darobactin 8
tggaactggtcaaaatgcttc

SEQ ID NO: 25 - protein, 7 aa, Artificial Sequence
  Synthetic Construct
WNWSKCF

SEQ ID NO: 26 - DNA, 21 bp, Artificial Sequence
  Propetide core sequence - Darobactin 9
  CDS (1)..(21): Propetide core sequence - Darobactin 9
tggaactggtcaaaaagctgg

SEQ ID NO: 27 - protein, 7 aa, Artificial Sequence
  Synthetic Construct
WNWSKSW

SEQ ID NO: 28 - DNA, 21 bp, Artificial Sequence
  Propetide core sequence - Darobactin 10
  CDS (1)..(21): Propetide core sequence - Darobactin 10
tggaactggtcaaaaagctac

SEQ ID NO: 29 - protein, 7 aa, Artificial Sequence
  Synthetic Construct
WNWSKSY

SEQ ID NO: 30 - DNA, 21 bp, Artificial Sequence
  Propetide core sequence - Darobactin 11
  CDS (1)..(21): Propetide core sequence - Darobactin 11
tggcagtggtcaaaaagcttc

SEQ ID NO: 31 - protein, 7 aa, Artificial Sequence
  Synthetic Construct
WQWSKSF

SEQ ID NO: 32 - DNA, 21 bp, Artificial Sequence
  Propetide core sequence - Darobactin 12
  CDS (1)..(21): Propetide core sequence - Darobactin 12
tggacctggtcaaaaagcttc

SEQ ID NO: 33 - protein, 7 aa, Artificial Sequence
  Synthetic Construct
WTWSKSF

SEQ ID NO: 34 - DNA, 21 bp, Artificial Sequence
  Propetide core sequence - Darobactin 13
  CDS (1)..(21): Propetide core sequence - Darobactin 13
tgggcatggtcaaaaagcttc

SEQ ID NO: 35 - protein, 7 aa, Artificial Sequence
  Synthetic Construct
WAWSKSF

SEQ ID NO: 36 - DNA, 21 bp, Artificial Sequence
  Propetide core sequence - Darobactin 14
  CDS (1)..(21): Propetide core sequence - Darobactin 14
tggaactgggcaaaaagcttc

SEQ ID NO: 37 - protein, 7 aa, Artificial Sequence
  Synthetic Construct
WNWAKSF

SEQ ID NO: 38 - DNA, 21 bp, Artificial Sequence
  Propetide core sequence - Darobactin 15
  CDS (1)..(21): Propetide core sequence - Darobactin 15
tggaactggtcagcaagcttc

SEQ ID NO: 39 - protein, 7 aa, Artificial Sequence
  Synthetic Construct
WNWSASF

SEQ ID NO: 40 - DNA, 21 bp, Artificial Sequence
  Propetide core sequence - Darobactin 16
  CDS (1)..(21): Propetide core sequence - Darobactin 16
tggaactggtcaaaagctttc

SEQ ID NO: 41 - protein, 7 aa, Artificial Sequence
  Synthetic Construct
WNWSKAF

SEQ ID NO: 42 - DNA, 21 bp, Artificial Sequence
  Propetide core sequence - Darobactin 17
  CDS (1)..(21): Propetide core sequence - Darobactin 17
tggaactggtcaaaaagcgca

SEQ ID NO: 43 - protein, 7 aa, Artificial Sequence
  Synthetic Construct
WNWSKSA

SEQ ID NO: 44 - DNA, 8131 bp, Artificial Sequence
  Biosynthetic Gene Cluster (BGC)
  misc_feature (1773)..(1775): n is a, g, c or t each nucleotide is a nucleotide which is part of a base triplet coding for an amino acid
  misc_feature (1779)..(1781): n is a, g, c or t each nucleotide is a nucleotide which is part of a base triplet coding for an amino acid
cgcgcgcgattctaaccggtataagaaataaagtggagcgccggcgattatccctaggtcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcataataccttaaatgaaaccgttaaaactcaagaagcactcaattctcttgctgcatcattcaaagagactgaactctcaattactgataaagcactaaacgaattaagcaataaacctaagatccctgagatcacggcctggaactggnnnaaannntggcaggaaatttaaagcttatcccatcaggttattttattttcctgaaaaaacaatgtccattgggataaacaatcaagttgttcagttaaaataaataattttttgaaccttaatcatagggtaatcctatgtattatccgggattaccctaaatactttcataaagtaaatataaaaatttgaataatacagataagtttttttaacctgaatggaaggtttgtattttatcattaaacacaaccaccttttaccactaatattattggattttttcatttcaagatgaaaatataatagatttcagtaactaaaaagggtaatagcctgtcccagcaggtgttactgatgcaggcaaataaaatagccctaataggatgggctttaagtaaggaacggtagccataactgcagtcctgaggatccccaccctgcaagggcccggaaccgcaaggttccgggcccttcgccttttatcaggttatttgcggccgctggacagcaagcgaaccggaattgccagctggggcgccctctggtaaggttgggaagccctgcaaagtaaactggatggctttcttgccgccaaggatctgatggcgcaggggatcaagatctgatcaagagacaggataaggaggtacagattatgaatgctattaacatctcagaatccatgaaacgtttcaatatattggtatcgatactctcagtatcaatagcaatattagcaatttatttactttctactgccttctattcagatgctcgagttgataactattatcaagataatggcaacatttaccgggtggaaaccacctttaatttgcccaatggagaaaaagtgagatcggcaaaatcgccattgccgctcatagacgagctagaaaaagataaaaggataaatcgtgcagactactttttcaaacttaacacgacgattgagttacaaggaaaaaagatcaccaaagttcccgtgtttgcagtcagtcatcaattcctgaataccctgtctccctttaagaaaatcgataatatcctgggagcaaatgagatttacatcacaaaagagttcaataatcgctatctcggcctggaaaatcccaaaggaaaatctattgttctggataatgaaacctatatcatcaaggatatcgtagaaaaacgtcatgattcgagtcttaatatgaatgctattatttcatttaagccaacacttatcaaaaactacaataaagaaatattcaattggtatgatacacatgcttatatttttataaaactctctgatgaaaaatcaatatccaatcgcgatgcgcttcttaatcatattattgcaaccaaagcccccaatcttccaggcgctccatttactgccagtgaatttattcacctatcattaaaaaaatcaaaagatattcattaccaagatggattgcctgatgaaatttctattggtttatcaaaggagataatttatagctcatatatcgcgttcgcatttatcttattttccacattaaccaactattataatttaacttctgctgattcagcagaaaaaagggatatttatcagttaaagaaagcgctaggcgcttcaactttcaatattatttgcgattcactgccggccatgttgatcaaaataatcctttccacccttatctttttgttacttatcataactatatttaatgtctctctctatataaataatatatttaccctattagaagcagacagtttaatatatctagggatattttcagttgttattgcctttatctttatttcatcaatacatttttcattctcattacgtttttatatttatcaaagaaacagaaaaatggatatgagatatgaatcaacccttgcctcttggtttaggaaatcatcattggctattcaattacttgtttccggaatatctatttatttagtcaccggattaaccaatcaatatattcaccttgttgatttacatactccttatgataatgccaagacaatatcaatagcaattaataacgataataataaaaaatcaactattgatgaattaagctatgagtttatcagaaaatacaatacttcaaaaataaccctcagtaattggcgaccatttgatatgtcaagagaaaatatcactattaattattccggccaacaaaacaggaataactatatctcttcaaatgttattactgcggatgaaaatttcactgatgtgtggagaatgaagatattagccggtggtgataaacgagtatatcagagtgataatgctgatgtagtccatgcattagtgacaaaagaatttttaaaacagaatggaattctcaaatatgatgatatttttaacaactactattggtacacaatggaggataaaaagatccaaatcagatttattcagattatagatgatttcaatttaggtgctgtagatgaaccattcaggccaatagttgttttcatcaaaaaagatcatggaaaatatgcttcgctgaatttaaacaatatgaaaaacctttctccggtacttaacgaactatcaaaacagggttttgataataatgaaattgatttaaccagtcacttatttaactcttattatgataattatttaaaagtaataaaattatcatctgccgtcgctattttctcggttctattattagtcatcgcctgctttactaccagcatgactgactttaacgcgatgaaaagggagttaagtataatggaatctattggtggttctatttacaccaatctcattcactttttatcgaaaagtgtcatcccgataatcgcatcaattttcgtggcatatatatcaggaagaactattttagtttatctgcttgatgaatatagtgtgatttacaccgattcttttgtatacagtgctatcgccctatctgctacaatgatattcaccgttattataatgatgactacctatttggtgaattatagaaaattatccgccactaaatttttgtgagattaattatggatatagaaatcagaaaaaaaggcatacataacagagagaaattactcattctcattttctttatcgcttttatttcatttgttgtatttattatctattatgtcgtttcggctaaagacgctttcgtgaatgaaaatgaagttacccagtttcaggtcagcagcaaagtagatagtgatattctgaaaaccagggcagttgtggttcctgataaaagtgtctctatctcaaccgagatagggggaatcgttaccgatataatgaaaaaaccatcacaagatgttttaaagaatgaagaaatcgtgaaattatctaattttaatttcacattgaataattcttcgatgttagcggacgtgacagataaactcaataatttaataaacataagaataaacttgcaatctgactatcgggatatcaataaccgattcttagaagccgccaaagaactaaaagaagtagaagataaattaaagcgatatcacgatcttgtacataaaaattatatttctaaagaaactatgtccgatttaaggattaaaagagattattggcacgatgtttatctattctacaaaaaattaaaaaatgataaagatcgtgatatatcaaaacagttaaaggaaatcgacgagtttgtcgaaaaacagagaaaactctctgacatcatcgaaaatggtttcgagcagctttcaataaaatcccctatcgccggtaatatcagttcattagatttaattttaggacaacggttaaaacctggcgataaaatagctattgttgatgatttatcaaacttttattttgaatcagaaatcaacgaatactatttaaacaaaataacataccattcatctgccagtttaatctataacaatggaaaaatacctttactggtgaaattaatttcatctgaagtgaataatggaacattcaaagtcagatttgaattggaagataaaaaaacaattaacttcaaacgggggcaatccgttgatgtcatgattaaacttgatgaagatcggaaaatattctccgtaccttcatctatggtgttttctattgacaataaaaactatgtgtttgtttaccatccagataaaaaaattgcccgacgtattcaagtttccaccggccaagataatggttctgatattgagatcaaaagtggtgttactgaaggacaaacgctcgttagctttggtaagaataaattagttaataatgatacagtaaggattgaataaaatgattagcatgatgaatatctgtaaatcatataaaacgaaattcattcaaacaaatgttttgaatgacataaatctcaatatagataaaggcgaatttatttctataatgggagcgtcagggtcagggaaatcaacattactgaatgtcattggcatgtttgaaacaatcgatagcggtcaattaacactgaacaatagcaatattttaacaatgaaatattctgaaaaaatatcattcagacgagaatttattggttatatttttcaatcattcaatctgttacccaatttgacggtatttgaaaacatagagttaccgttaaaatacagaggttttcctaaaaaacagcggaaaagcaaagtatttgatgcaataaatagttttggattagaaaatcgggaaaatcataaaccgatacaattatctggtggacaacagcaacgtgttgctatcgccagagcaatgatagcaaaccccaccattttattagctgatgaaccaacggggaacctggatagcgtaaatggtcagaatattttatcttcacttaaggagctgaatgaaaacggtacgacgattgttatggtaacccattccgtagaagcggcagcgttttctgacagaatattaacgatgagggatggccacttgcttgtttgagcataaataacataataaaggagttctttatggacacaataatccccataaaatatttagattcagacgaatcatcgattcttaagaaatcatctaaaattaactacaggcaattagcttgcagaattatcggtgaaatctccgccgaaaaaatattagatgatgatgaactggctttatataataaagaaatcagtatacatttcagccctgaaattattaatgctaataaattagttgtggttgtgaaagccaccaggctttgcaatttaagatgcacttattgtcactcctgggcagaaggaaaaggaaataccttaacattcttcaatttaatgcgttccattcaccgtttcttatccctaccgaatatcaagcgatttgaattcgtctggcatggcggcgaagtaacgttgttgaatgttaattactttaagaaactcatctggttacaggaacaatttaaaaaaccggatcaagttatcaccaattcggtacagacaaatgccgtcaatattcctgaagattggttagtgttcctcaaaggtattggaatgggggtaggaataagcgttgatggtattccggaaatacacgatagcaggagattagattacagaggaaggccaacatcccataaagtcgcggcaagtatgaaaaagttaagaagttatggcataccttacggtgcgcttatcgtcgtcgaccgcgatgtttatgaatcaaatatagaaaaaatgctctcttatttttacgaaatcggtttaacggatattgaatttctgaatattgtcccagataaccgatgccagccgggtgatgatcctggaggaagttatataacttaccataactatattaatttcctttctaaggttttccgtgtctggtggaatggttatcaaggcaaaatcaatattcgcttgtttgacggatttattgacagtatcaaatcgtcccaaaagaaaatgtcagattgttattgggcgggtaactgttctcaggaaataatcacattagaacctaatggtacggtatcagcatgtgataaatatgttggtgctgaagggaataattatggttcgattattgataatgatcttgggaatttactatctaaatcaaatacaaataaggatcatcttaaagaggaaatggaatcttatgaaaaaatgcatcaatgtaaatggtttcatttgtgtaatggtggatgcccacacgatcgagtgaccaacaggaagcacaatccaaattatgatggttcatgttgtggaaccggcggtttgttggagacaataaaacaaaccatcgcggcgtaaattctaactagttcctgattaattaaccaccctgcaagggcccggaaccgcaaggttccgggcccttcgccttttcgtcgacg

SEQ ID NO: 45 - DNA, 20 bp, Artificial Sequence
  Forward Primer - pr1dar_fw for overlap extension PCR
ggtgatgtcggcgatatagg

SEQ ID NO: 46 - DNA, 27 bp, Artificial Sequence
  Forward Primer - pr3dartrp_fw for overlap extension PCR
tggcaggaaatttaaagcttatcccat

SEQ ID NO: 47 - DNA, 21 bp, Artificial Sequence
  Reverse Primer - pr4dar_rv for overlap extension PCR
tggggatcctcaggactgcag

SEQ ID NO: 48 - DNA, 30 bp, Artificial Sequence
  Reverse Primer - pr2dar22_rv containing mutations of D22
aaatttcctgccaccgtttagtccagttcc

SEQ ID NO: 49 - DNA, 30 bp, Artificial Sequence
  Reverse Primer - pr2dar23_rv containing mutations of D23
aaatttcctgccaatgtttagtccagttcc

SEQ ID NO: 50 - DNA, 30 bp, Artificial Sequence
  Reverse Primer - pr2dar24_rv containing mutations of D24
aaatttcctgccatgatttagtccagttcc

SEQ ID NO: 51 - DNA, 30 bp, Artificial Sequence
  Reverse Primer - pr2dar25_rv containing mutations of D25
aaatttcctgccacgctttagtccagttcc

SEQ ID NO: 52 - DNA, 30 bp, Artificial Sequence
  Reverse Primer - pr2dar30_rv containing mutations of D30
aaatttcctgccatgatttcgcccagttcc

SEQ ID NO: 53 - DNA, 30 bp, Artificial Sequence
  Reverse Primer - pr2dar31_rv containing mutations of D31
aaatttcctgccaccgtttcgcccagttcc

SEQ ID NO: 54 - DNA, 30 bp, Artificial Sequence
  Reverse Primer - pr2dar32_rv containing mutations of D32
aaatttcctgccaccgtttacaccagttcc

SEQ ID NO: 55 - DNA, 30 bp, Artificial Sequence
  Reverse Primer - pr2dar33_rv containing mutations of D33
aaatttcctgccatgatttacaccagttcc

SEQ ID NO: 56 - DNA, 30 bp, Artificial Sequence
  Reverse Primer - pr2dar34_rv containing mutations of D34
aaatttcctgccacgctttacaccagttcc

SEQ ID NO: 57 - DNA, 30 bp, Artificial Sequence
  Reverse Primer - pr2dar36_rv containing mutations of D36
aaatttcctgccattttttagtccagttcc

SEQ ID NO: 58 - DNA, 30 bp, Artificial Sequence
  Reverse Primer - pr2dar37_rv containing mutations of D37
aaatttcctgccaagtttttgaccagttcc

SEQ ID NO: 59 - DNA, 30 bp, Artificial Sequence
  Reverse Primer - pr2dar38_rv containing mutations of D38
aaatttcctgccaagttttagtccagttcc

SEQ ID NO: 60 - DNA, 21 bp, Artificial Sequence
  Propetide core sequence - Darobactin 9
  CDS (1)..(21): Propetide core sequence - Darobactin 9
tggaactggtcaaaaagctgg

SEQ ID NO: 61 - protein, 7 aa, Artificial Sequence
  Synthetic Construct
WNWSKSW

SEQ ID NO: 62 - DNA, 21 bp, Artificial Sequence
  Propetide core sequence
  CDS (1)..(21): Propetide core sequence - Darobactin 22
tggaactggactaaacggtgg

SEQ ID NO: 63 - protein, 7 aa, Artificial Sequence
  Synthetic Construct
WNWTKRW

SEQ ID NO: 64 - DNA, 21 bp, Artificial Sequence
  Propetide core sequence
  CDS (1)..(21): Propetide core sequence - Darobactin 23
tggaactggactaaacattgg

SEQ ID NO: 65 - protein, 7 aa, Artificial Sequence
  Synthetic Construct
WNWTKHW

SEQ ID NO: 66 - DNA, 21 bp, Artificial Sequence
  Propetide core sequence
  CDS (1)..(21): Propetide core sequence - Darobactin 24
tggaactggactaaatcatgg

SEQ ID NO: 67 - protein, 7 aa, Artificial Sequence
  Synthetic Construct
WNWTKSW

SEQ ID NO: 68 - DNA, 21 bp, Artificial Sequence
  Propetide core sequence
  CDS (1)..(21): Propetide core sequence - Darobactin 25
tggaactggactaaagcgtgg

SEQ ID NO: 69 - protein, 7 aa, Artificial Sequence
  Synthetic Construct
WNWTKAW

SEQ ID NO: 70 - DNA, 21 bp, Artificial Sequence
  Propetide core sequence
  CDS (1)..(21): Propetide core sequence - Darobactin 30
tggaactgggcgaaatcatgg

SEQ ID NO: 71 - protein, 7 aa, Artificial Sequence
  Synthetic Construct
WNWAKSW

SEQ ID NO: 72 - DNA, 21 bp, Artificial Sequence
  Propetide core sequence
  CDS (1)..(21): Propetide core sequence - Darobactin 31
tggaactgggcgaaacggtgg

SEQ ID NO: 73 - protein, 7 aa, Artificial Sequence
  Synthetic Construct
WNWAKRW

SEQ ID NO: 74 - DNA, 21 bp, Artificial Sequence
  Propetide core sequence
  CDS (1)..(21): Propetide core sequence - Darobactin 32
tggaactggtgtaaacggtgg

SEQ ID NO: 75 - protein, 7 aa, Artificial Sequence
  Synthetic Construct
WNWCKRW

SEQ ID NO: 76 - DNA, 21 bp, Artificial Sequence
  Propetide core sequence
  CDS (1)..(21): Propetide core sequence - Darobactin 33
tggaactggtgtaaatcatgg

SEQ ID NO: 77 - protein, 7 aa, Artificial Sequence
  Synthetic Construct
WNWCKSW

SEQ ID NO: 78 - DNA, 21 bp, Artificial Sequence
  Propetide core sequence
  CDS (1)..(21): Propetide core sequence - Darobactin 34
tggaactggtgtaaagcgtgg

SEQ ID NO: 79 - protein, 7 aa, Artificial Sequence
  Synthetic Construct
WNWCKAW

SEQ ID NO: 80 - DNA, 21 bp, Artificial Sequence
  Propetide core sequence
  CDS (1)..(21): Propetide core sequence - Darobactin 36
tggaactggactaaaaaatgg

SEQ ID NO: 81 - protein, 7 aa, Artificial Sequence
  Synthetic Construct
WNWTKKW

SEQ ID NO: 82 - DNA, 21 bp, Artificial Sequence
  Propetide core sequence
  CDS (1)..(21): Propetide core sequence - Darobactin 37
tggaactggtcaaaaacttgg

SEQ ID NO: 83 - protein, 7 aa, Artificial Sequence
  Synthetic Construct
WNWSKTW

SEQ ID NO: 84 - DNA, 21 bp, Artificial Sequence
  Propetide core sequence
  CDS (1)..(21): Propetide core sequence - Darobactin 38
tggaactggactaaaacttgg

SEQ ID NO: 85 - protein, 7 aa, Artificial Sequence
  Synthetic Construct
WNWTKTW

SEQ ID NO: 86 - DNA, 8131 bp, Artificial Sequence
  Biosynthetic Gene Cluster (BGC)
  misc_feature (1773)..(1775): n is a, g, c or t each nucleotide is a nucleotide which is part of a base triplet coding for an amino acid
  misc_feature (1776)..(1778): each nucleotide is a nucleotide which is part of a base triplet coding for arginine or lysine, including agg, aga, aag, aaa, cga, cgc, cgg and cgt
  misc_feature (1779)..(1781): n is a, g, c or t each nucleotide is a nucleotide which is part of a base triplet coding for an amino acid
  misc_feature (1782)..(1784): each nucleotide is a nucleotide which is part of a base triplet coding for tryptophan, alanine or histidine, including tgg, gca, gcc, gcg, gct, cac and cat
cgcgcgcgattctaaccggtataagaaataaagtggagcgccggcgattatccctaggtcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcataataccttaaatgaaaccgttaaaactcaagaagcactcaattctcttgctgcatcattcaaagagactgaactctcaattactgataaagcactaaacgaattaagcaataaacctaagatccctgagatcacggcctggaactggnnnnnnnnnnnncaggaaatttaaagcttatcccatcaggttattttattttcctgaaaaaacaatgtccattgggataaacaatcaagttgttcagttaaaataaataattttttgaaccttaatcatagggtaatcctatgtattatccgggattaccctaaatactttcataaagtaaatataaaaatttgaataatacagataagtttttttaacctgaatggaaggtttgtattttatcattaaacacaaccaccttttaccactaatattattggattttttcatttcaagatgaaaatataatagatttcagtaactaaaaagggtaatagcctgtcccagcaggtgttactgatgcaggcaaataaaatagccctaataggatgggctttaagtaaggaacggtagccataactgcagtcctgaggatccccaccctgcaagggcccggaaccgcaaggttccgggcccttcgccttttatcaggttatttgcggccgctggacagcaagcgaaccggaattgccagctggggcgccctctggtaaggttgggaagccctgcaaagtaaactggatggctttcttgccgccaaggatctgatggcgcaggggatcaagatctgatcaagagacaggataaggaggtacagattatgaatgctattaacatctcagaatccatgaaacgtttcaatatattggtatcgatactctcagtatcaatagcaatattagcaatttatttactttctactgccttctattcagatgctcgagttgataactattatcaagataatggcaacatttaccgggtggaaaccacctttaatttgcccaatggagaaaaagtgagatcggcaaaatcgccattgccgctcatagacgagctagaaaaagataaaaggataaatcgtgcagactactttttcaaacttaacacgacgattgagttacaaggaaaaaagatcaccaaagttcccgtgtttgcagtcagtcatcaattcctgaataccctgtctccctttaagaaaatcgataatatcctgggagcaaatgagatttacatcacaaaagagttcaataatcgctatctcggcctggaaaatcccaaaggaaaatctattgttctggataatgaaacctatatcatcaaggatatcgtagaaaaacgtcatgattcgagtcttaatatgaatgctattatttcatttaagccaacacttatcaaaaactacaataaagaaatattcaattggtatgatacacatgcttatatttttataaaactctctgatgaaaaatcaatatccaatcgcgatgcgcttcttaatcatattattgcaaccaaagcccccaatcttccaggcgctccatttactgccagtgaatttattcacctatcattaaaaaaatcaaaagatattcattaccaagatggattgcctgatgaaatttctattggtttatcaaaggagataatttatagctcatatatcgcgttcgcatttatcttattttccacattaaccaactattataatttaacttctgctgattcagcagaaaaaagggatatttatcagttaaagaaagcgctaggcgcttcaactttcaatattatttgcgattcactgccggccatgttgatcaaaataatcctttccacccttatctttttgttacttatcataactatatttaatgtctctctctatataaataatatatttaccctattagaagcagacagtttaatatatctagggatattttcagttgttattgcctttatctttatttcatcaatacatttttcattctcattacgtttttatatttatcaaagaaacagaaaaatggatatgagatatgaatcaacccttgcctcttggtttaggaaatcatcattggctattcaattacttgtttccggaatatctatttatttagtcaccggattaaccaatcaatatattcaccttgttgatttacatactccttatgataatgccaagacaatatcaatagcaattaataacgataataataaaaaatcaactattgatgaattaagctatgagtttatcagaaaatacaatacttcaaaaataaccctcagtaattggcgaccatttgatatgtcaagagaaaatatcactattaattattccggccaacaaaacaggaataactatatctcttcaaatgttattactgcggatgaaaatttcactgatgtgtggagaatgaagatattagccggtggtgataaacgagtatatcagagtgataatgctgatgtagtccatgcattagtgacaaaagaatttttaaaacagaatggaattctcaaatatgatgatatttttaacaactactattggtacacaatggaggataaaaagatccaaatcagatttattcagattatagatgatttcaatttaggtgctgtagatgaaccattcaggccaatagttgttttcatcaaaaaagatcatggaaaatatgcttcgctgaatttaaacaatatgaaaaacctttctccggtacttaacgaactatcaaaacagggttttgataataatgaaattgatttaaccagtcacttatttaactcttattatgataattatttaaaagtaataaaattatcatctgccgtcgctattttctcggttctattattagtcatcgcctgctttactaccagcatgactgactttaacgcgatgaaaagggagttaagtataatggaatctattggtggttctatttacaccaatctcattcactttttatcgaaaagtgtcatcccgataatcgcatcaattttcgtggcatatatatcaggaagaactattttagtttatctgcttgatgaatatagtgtgatttacaccgattcttttgtatacagtgctatcgccctatctgctacaatgatattcaccgttattataatgatgactacctatttggtgaattatagaaaattatccgccactaaatttttgtgagattaattatggatatagaaatcagaaaaaaaggcatacataacagagagaaattactcattctcattttctttatcgcttttatttcatttgttgtatttattatctattatgtcgtttcggctaaagacgctttcgtgaatgaaaatgaagttacccagtttcaggtcagcagcaaagtagatagtgatattctgaaaaccagggcagttgtggttcctgataaaagtgtctctatctcaaccgagatagggggaatcgttaccgatataatgaaaaaaccatcacaagatgttttaaagaatgaagaaatcgtgaaattatctaattttaatttcacattgaataattcttcgatgttagcggacgtgacagataaactcaataatttaataaacataagaataaacttgcaatctgactatcgggatatcaataaccgattcttagaagccgccaaagaactaaaagaagtagaagataaattaaagcgatatcacgatcttgtacataaaaattatatttctaaagaaactatgtccgatttaaggattaaaagagattattggcacgatgtttatctattctacaaaaaattaaaaaatgataaagatcgtgatatatcaaaacagttaaaggaaatcgacgagtttgtcgaaaaacagagaaaactctctgacatcatcgaaaatggtttcgagcagctttcaataaaatcccctatcgccggtaatatcagttcattagatttaattttaggacaacggttaaaacctggcgataaaatagctattgttgatgatttatcaaacttttattttgaatcagaaatcaacgaatactatttaaacaaaataacataccattcatctgccagtttaatctataacaatggaaaaatacctttactggtgaaattaatttcatctgaagtgaataatggaacattcaaagtcagatttgaattggaagataaaaaaacaattaacttcaaacgggggcaatccgttgatgtcatgattaaacttgatgaagatcggaaaatattctccgtaccttcatctatggtgttttctattgacaataaaaactatgtgtttgtttaccatccagataaaaaaattgcccgacgtattcaagtttccaccggccaagataatggttctgatattgagatcaaaagtggtgttactgaaggacaaacgctcgttagctttggtaagaataaattagttaataatgatacagtaaggattgaataaaatgattagcatgatgaatatctgtaaatcatataaaacgaaattcattcaaacaaatgttttgaatgacataaatctcaatatagataaaggcgaatttatttctataatgggagcgtcagggtcagggaaatcaacattactgaatgtcattggcatgtttgaaacaatcgatagcggtcaattaacactgaacaatagcaatattttaacaatgaaatattctgaaaaaatatcattcagacgagaatttattggttatatttttcaatcattcaatctgttacccaatttgacggtatttgaaaacatagagttaccgttaaaatacagaggttttcctaaaaaacagcggaaaagcaaagtatttgatgcaataaatagttttggattagaaaatcgggaaaatcataaaccgatacaattatctggtggacaacagcaacgtgttgctatcgccagagcaatgatagcaaaccccaccattttattagctgatgaaccaacggggaacctggatagcgtaaatggtcagaatattttatcttcacttaaggagctgaatgaaaacggtacgacgattgttatggtaacccattccgtagaagcggcagcgttttctgacagaatattaacgatgagggatggccacttgcttgtttgagcataaataacataataaaggagttctttatggacacaataatccccataaaatatttagattcagacgaatcatcgattcttaagaaatcatctaaaattaactacaggcaattagcttgcagaattatcggtgaaatctccgccgaaaaaatattagatgatgatgaactggctttatataataaagaaatcagtatacatttcagccctgaaattattaatgctaataaattagttgtggttgtgaaagccaccaggctttgcaatttaagatgcacttattgtcactcctgggcagaaggaaaaggaaataccttaacattcttcaatttaatgcgttccattcaccgtttcttatccctaccgaatatcaagcgatttgaattcgtctggcatggcggcgaagtaacgttgttgaatgttaattactttaagaaactcatctggttacaggaacaatttaaaaaaccggatcaagttatcaccaattcggtacagacaaatgccgtcaatattcctgaagattggttagtgttcctcaaaggtattggaatgggggtaggaataagcgttgatggtattccggaaatacacgatagcaggagattagattacagaggaaggccaacatcccataaagtcgcggcaagtatgaaaaagttaagaagttatggcataccttacggtgcgcttatcgtcgtcgaccgcgatgtttatgaatcaaatatagaaaaaatgctctcttatttttacgaaatcggtttaacggatattgaatttctgaatattgtcccagataaccgatgccagccgggtgatgatcctggaggaagttatataacttaccataactatattaatttcctttctaaggttttccgtgtctggtggaatggttatcaaggcaaaatcaatattcgcttgtttgacggatttattgacagtatcaaatcgtcccaaaagaaaatgtcagattgttattgggcgggtaactgttctcaggaaataatcacattagaacctaatggtacggtatcagcatgtgataaatatgttggtgctgaagggaataattatggttcgattattgataatgatcttgggaatttactatctaaatcaaatacaaataaggatcatcttaaagaggaaatggaatcttatgaaaaaatgcatcaatgtaaatggtttcatttgtgtaatggtggatgcccacacgatcgagtgaccaacaggaagcacaatccaaattatgatggttcatgttgtggaaccggcggtttgttggagacaataaaacaaaccatcgcggcgtaaattctaactagttcctgattaattaaccaccctgcaagggcccggaaccgcaaggttccgggcccttcgccttttcgtcgacg

SEQ ID NO: 87 - DNA, 21 bp, Artificial Sequence
  Peptide Derivative - Darobactin 39
  CDS (1)..(21): Propetide core sequence - Darobactin 39
tggaactggactcggcggtgg

SEQ ID NO: 88 - protein, 7 aa, Artificial Sequence
  Synthetic Construct
WNWTRRW

SEQ ID NO: 89 - DNA, 31 bp, Artificial Sequence
  Reverse Primer - pr2dar39_rv containing mutations of D39
aaatttcctgccaccgccgagtccagttcca

(19) United States
(12) Patent Application Publication
(10) Pub. No.: US 2025/0276998 A1
(43) Pub. Date: Sep. 4, 2025

(54) NOVEL DAROBACTIN DERIVATIVES

(71) Applicant: Helmholtz-Zentrum Fur Infektionsforschung GmbH, Braunschweig (DE)

(72) Inventors: Carsten E. Seyfert, Braunschweig (DE); Fabian T. Panter, Braunschweig (DE); Rolf Müller, Braunschweig (DE); Selina Deckarm, Braunschweig (DE); Sebastian Gross, Braunschweig (DE); Domen Scherzer, Braunschweig (DE); Chantal Bader, Braunschweig (DE); Jennifer Herrmann, Braunschweig (DE)

(21) Appl. No.: 18/277,870

(22) PCT Filed: Feb. 18, 2022
(86) PCT No.: PCT/EP2022/054063

§ 371 Date: Aug. 18, 2023

(30) Foreign Application Priority Data
Feb. 19, 2021 (EP) 21158189.7
Jul. 22, 2021 (EP) 21187173.6

(51) Int. Cl. C07K 7/06; A61K 38/08; A61P 31/04; C12N 15/70

(52) U.S. Cl. CPC C07K 7/06; A61K 38/08; A61P 31/04; C12N 15/70

(57) ABSTRACT

The present invention relates to darobactin derivatives of formula (I), methods of their use, and a method of production.

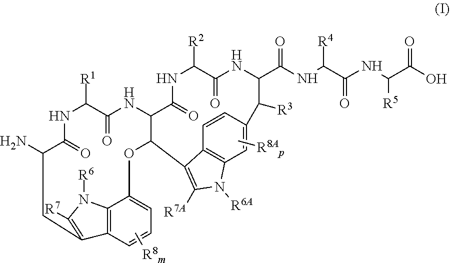

### CROSS-REFERENCE TO RELATED APPLICATIONS

This application is a U.S. national phase application, pursuant to 35 U.S.C. § 371, of PCT Application No. PCT/EP2022/054063, filed Feb. 18, 2022, which claims priority from European Patent Application No. 21158189.7, filed on Feb. 19, 2021 and European Application No. 21187173.6, filed on Jul. 22, 2021. The entire contents of these applications are incorporated herein by reference in their entirety.

### INCORPORATION BY REFERENCE OF SEQUENCE LISTING

The instant application contains a Sequence Listing which has been submitted electronically in ASCII format and is hereby incorporated by reference in its entirety. Said ASCII copy, created on Feb. 19, 2024, is named 348710_01501_SequenceListing.txt and is 58,400 bytes in size.

The present invention relates to novel derivatives of darobactin, methods of their use, and a method of production.

The emerging number of antibiotic-resistant bacterial pathogens leads to increasing mortality rates in humans. Many deaths are caused by the especially troublesome bacteria belonging to the ESKAPE panel (vancomycin-resistant Enterococci, Staphylococcus aureus, Klebsiella pneumonia, Acinetobacter baumannii, Pseudomonas aeruginosa and Enterobacter species). The World Health Organization recommended focusing on the discovery and development of new antibiotics with anti-Gram-negative activity.

Darobactin A is a ribosomally-produced peptide antibiotic that selectively kills Gram-negative pathogens, including bacteria from the ESKAPE panel (Imai, Y., Meyer, K. J., Iinishi, A. et al. A new antibiotic selectively kills Gram-negative pathogens. Nature 576, 459-464 (2019); WO 2020/018173). However, the production yield of Darobactin A in hitherto known producer strains is poor, which limits the possibilities to perform structure engineering or isolate sufficient amounts of compound needed for potential semi-synthesis approaches.

In one aspect, the present invention provides compounds of formula (I) or a salt thereof:

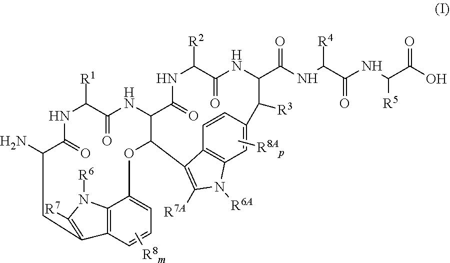

wherein
R¹, R², R⁴ and R⁵ are independently selected from H, CH₃, CH₂OH, one of the following groups:

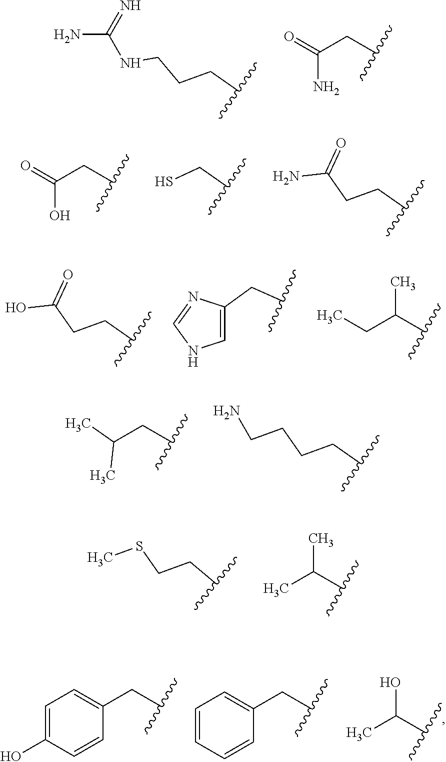

—CH₂—SR^{1A}, wherein R^{1A} is independently selected from an alkyl, an alkenyl, an alkynyl, a heteroalkyl, a cycloalkyl, a heterocycloalkyl, an alkylcycloalkyl, a heteroalkylcycloalkyl, an aryl, a heteroaryl, an aralkyl or a heteroaralkyl group, all of which groups may optionally be substituted;
or —CH₂-Ind, wherein Ind is an optionally substituted indole group;
R³ is selected from H, OH, SH, COOH, CONH₂, one of the following groups:

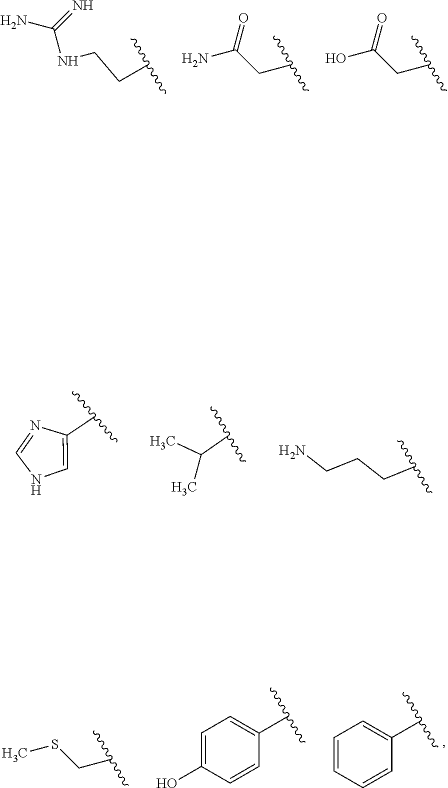

—SR^{3A}, wherein R^{3A} is selected from an alkyl, an alkenyl, an alkynyl, a heteroalkyl, a cycloalkyl, a heterocycloalkyl, an alkylcycloalkyl, a heteroalkylcycloalkyl, an aryl, a heteroaryl, an aralkyl or a heteroaralkyl group, all of which groups may optionally be substituted;
or -Ind, wherein Ind is an optionally substituted indole group;
R⁶ is a hydrogen atom or a methyl group (especially a hydrogen atom);
R⁷ is a hydrogen atom, a halogen atom, a hydroxy group or a methoxy group (especially a hydrogen atom);
R⁸ is, at each occasion, independently selected from a halogen atom, a hydroxy group or a methoxy group; and
m is an integer of from 0 to 3 (especially 0 or 1; preferably 0);
R^{6A} is a hydrogen atom or a methyl group (especially a hydrogen atom);
R^{7A} is a hydrogen atom, a halogen atom, a hydroxy group or a methoxy group (especially a hydrogen atom);
R^{8A} is, at each occasion, independently selected from a halogen atom, a hydroxy group or a methoxy group; and
p is an integer of from 0 to 3 (especially 0 or 1; preferably 0);
with the proviso, that R⁵ is not a group of formula —CH₂-phenyl if R⁴ is a group of formula —CH₂—OH or —CH₂—CH₂—CH₂—NH—C(═NH)—NH₂.

Preferably, R⁵ is selected from H, CH₃, CH₂OH or one of the following groups:

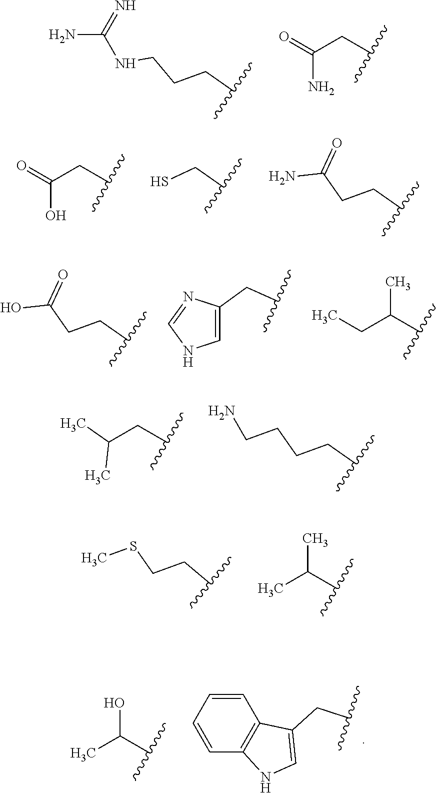

Further preferably, R⁵ is a group of formula —CH₂-Ind, wherein Ind is an optionally substituted indole group.

Moreover preferably, R⁵ is a group of formula —CH₃.

Further preferably, R⁵ is a group of the following formula:

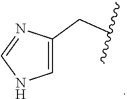 [SMILES: *Cc1c[nH]cn1]

Moreover preferably, the group of formula —CH₂-Ind is a group of the following formula:

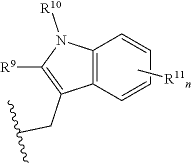

wherein R⁹ is a hydrogen atom, a halogen atom, a hydroxy group or a methoxy group (especially a hydrogen atom); R¹⁰ is a hydrogen atom or a methyl group (especially a hydrogen atom); R¹¹ is, at each occasion, independently selected from a halogen atom, a hydroxy group or a methoxy group and n is an integer of from 0 to 4 (especially 0 or 1; preferably 0).

Especially preferably, R⁵ is a group of the following formula:

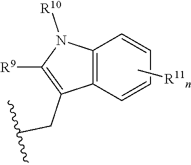

wherein R⁹ is a hydrogen atom, a halogen atom, a hydroxy group or a methoxy group (especially a hydrogen atom); R¹⁰ is a hydrogen atom or a methyl group (especially a hydrogen atom); R¹¹ is, at each occasion, independently selected from a halogen atom, a hydroxy group or a methoxy group and n is an integer of from 0 to 4 (especially 0 or 1; preferably 0).

In some preferred embodiments, R⁵ is a group of the following formula:

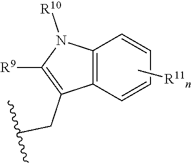

wherein R⁹ is a hydrogen or halogen atom (especially a hydrogen atom); R¹⁰ is a hydrogen atom or a methyl group (especially a hydrogen atom); R¹¹ is, at each occasion, independently selected from a halogen atom (especially fluor or chlor) and n is an integer of from 0 to 4 (especially 0 or 1; preferably 0).

Further especially preferably, R⁵ is a group of the following formula:

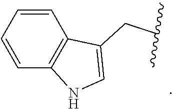 [SMILES: *Cc1c[nH]c2ccccc12]

In some embodiments, the group of formula —CH₂—SR^{1A} can preferably be:

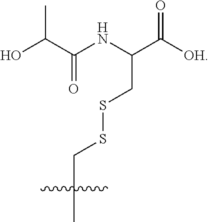 [SMILES: *CSSCC(NC(=O)C(C)O)C(=O)O]

Further preferably, R¹ is selected from the following groups: —CH₂—CONH₂, —CH₂—OH, —CH₂—CH₂—CONH₂ and —CH₂—CH(CH₃)—OH; and more preferably R¹ is a group of —CH₂—CONH₂.

Preferably, R² is selected from CH₃, CH₂OH, one of the following groups:

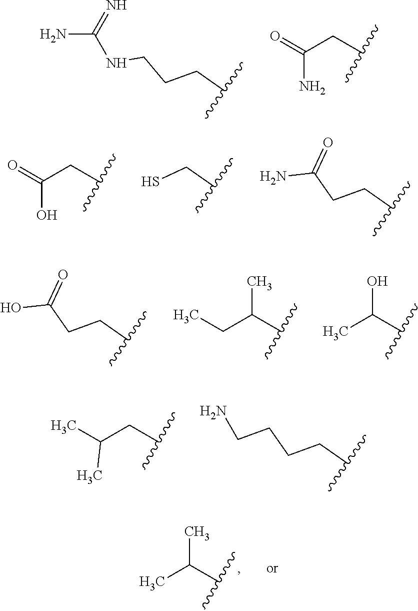

—CH₂—SR^{1A}, wherein R^{1A} is independently selected from an alkyl, an alkenyl, an alkynyl, a heteroalkyl, a cycloalkyl, a heterocycloalkyl, an alkylcycloalkyl, a heteroalkylcycloalkyl, an aryl, a heteroaryl, an aralkyl or a heteroaralkyl group, all of which groups may optionally be substituted.

Moreover preferably, R² is selected from the following groups: —CH₃, —CH₂—OH, —CH₂—SH, —CH₂—CONH₂, —CH₂—CH₂—CONH₂, —CH₂—CH₂—CH₂—NH—C(═NH)—NH₂, and —CH₂—CH(CH₃)—OH, such as —CH₃, —CH₂—OH, —CH₂—SH, and —CH₂—CH(CH₃)—OH, or such as —CH₂—OH and —CH₂—CH(CH₃)—OH.

Moreover preferably, R² is selected from the following groups: —CH₂—OH, —CH₂—CH(CH₃)—OH and —CH₃, or represents a group:

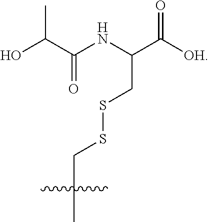 [SMILES: *CSSCC(NC(=O)C(C)O)C(=O)O]

Further preferably, R³ is a hydrogen atom or selected from the following groups: —CH₂—CH₂—CH₂—NH₂ and —CH₂—CH₂—NH—C(═NH)—NH₂. In some preferred embodiments, R³ is a group: —CH₂—CH₂—CH₂—NH₂ or —CH₂—CH₂—NH—C(═NH)—NH₂, preferably the group: —CH₂—CH₂—CH₂—NH₂.

R⁴ can preferably represent a hydrogen atom, CH₃, CH₂OH, one of the following groups:

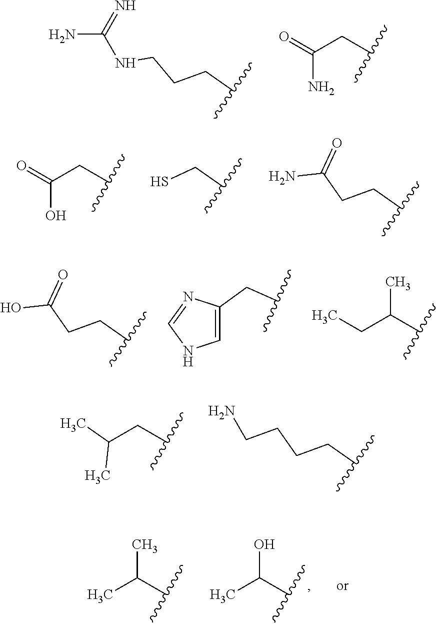

—CH₂—SR^{1A}, wherein R^{1A} is independently selected from an alkyl, an alkenyl, an alkynyl, a heteroalkyl, a cycloalkyl, a heterocycloalkyl, an alkylcycloalkyl, a heteroalkylcycloalkyl, an aryl, a heteroaryl, an aralkyl or a heteroaralkyl group, all of which groups may optionally be substituted (e.g. a group:

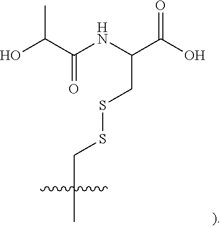 [SMILES: *CSSCC(NC(=O)C(C)O)C(=O)O]

Further preferably, R⁴ is CH₃, CH₂OH, or selected from one of the following groups:

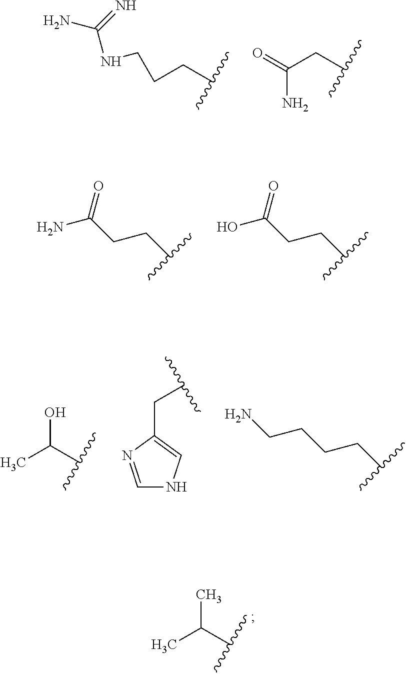

such as CH₃, CH₂OH, —CH₂—CH₂—CH₂—NH₂, —CH₂—CH₂—NH—C(═NH)—NH₂, —CH₂—CH(CH₃)—OH or

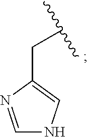 [SMILES: *Cc1c[nH]cn1]

or such as CH₃, CH₂OH, —CH₂—CH₂—NH—C(═NH)—NH₂, or —CH₂—CH(CH₃)—OH.

Moreover preferably, R⁴ is selected from the following groups: —CH₂—OH, —CH₃ and —CH₂—CH₂—CH₂—NH—C(═NH)—NH₂.

Further preferably, R⁴ is a group of formula —CH₂—SR^{4A}, wherein R^{4A} is selected from a hydrogen atom, an alkyl, an alkenyl, an alkynyl, a heteroalkyl, a cycloalkyl, a heterocycloalkyl, an alkylcycloalkyl, a heteroalkylcycloalkyl, an aryl, a heteroaryl, an aralkyl or a heteroaralkyl group, all of which groups may optionally be substituted. A preferred example of a group of formula —CH₂—SR^{4A} is:

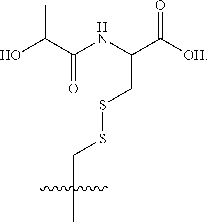 [SMILES: *CSSCC(NC(=O)C(C)O)C(=O)O]

In some embodiments, R⁶ is a methyl group or a hydrogen atom, preferably a hydrogen atom. In some embodiments, R^{6A} is a methyl group or a hydrogen atom, preferably a hydrogen atom. In some embodiments, each of R⁶ and R^{6A} is a hydrogen atom.

In some embodiments, R⁷ is a hydrogen or halogen atom, preferably a hydrogen atom.

In some embodiments, R^{7A} is a hydrogen or halogen atom, preferably a hydrogen atom.

In some embodiments, each of R⁷ and R^{7A} is a hydrogen atom.

In some embodiments, each of R⁶, R^{6A}, R⁷, and R^{7A} is a hydrogen atom.

In some embodiments, R⁸ is a halogen atom, preferably a fluor or chlor atom, and more preferably a fluor atom. In some embodiments, R^{8A} is a halogen atom, preferably a fluor or chlor atom, and more preferably a fluor atom. In some embodiments, one of R⁸ and R^{8A} is present.

In some embodiments, m is 0, 1, or 2, preferably 0 or 1, more preferably 0.

In some embodiments, p is 0, 1, or 2, preferably 0 or 1, more preferably 0.

In some embodiments, n is 0, 1, 2 or 3, preferably 0, 1 or 2, more preferably 0 or 1, especially 0.

Moreover preferred are compounds of formula (Ia) or a salt thereof:

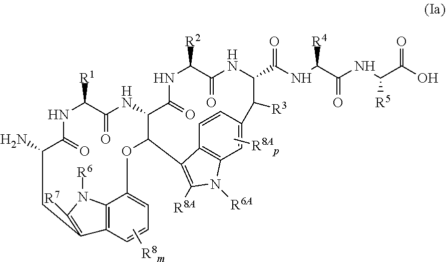

wherein R¹, R², R³, R⁴, R⁵, R⁶, R⁷, R⁸, R^{6A}, R^{7A}, R^{8A}, m and p are as defined above.

Further preferred are compounds of formula (Ia′) or a salt thereof:

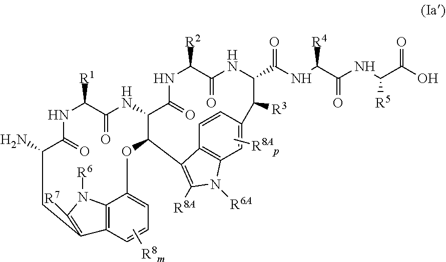

wherein R¹, R², R³, R⁴, R⁵, R⁶, R⁷, R⁸, R^{6A}, R^{7A}, R^{8A}, m and p are as defined above.

Moreover preferred are compounds of formula (Ib) or a salt thereof:

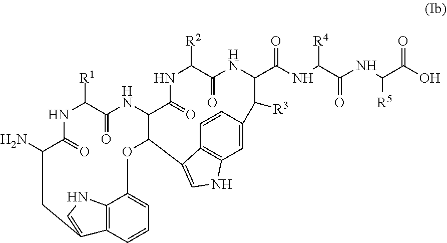

wherein R¹, R², R³, R⁴ and R⁵ are as defined above.

Further preferred are compounds of formula (Ib′) or a salt thereof:

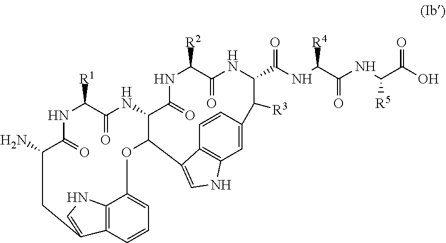

wherein R¹, R², R³, R⁴ and R⁵ are as defined above.

Moreover preferred are compounds of formula (Ib″) or a salt thereof:

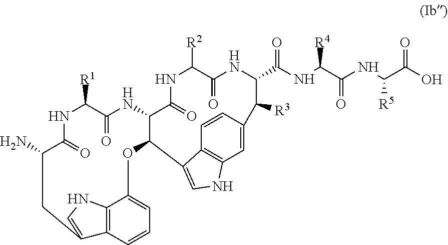

wherein R¹, R², R³, R⁴ and R⁵ are as defined above.

In some embodiments, the compound of formula (I) is a compound of formula (1):

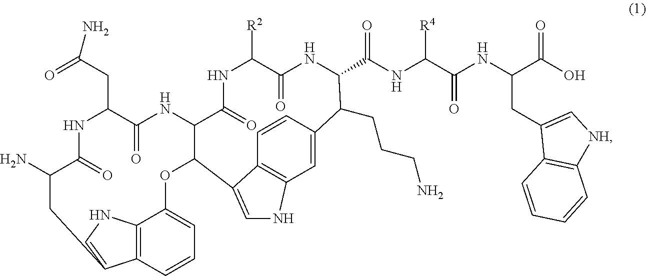

or a salt (especially a pharmaceutically acceptable salt) thereof, wherein R² and R⁴ are as defined above.

In some embodiments, the compound of formula (1) is a compound of formula (1a) or formula (1a′):

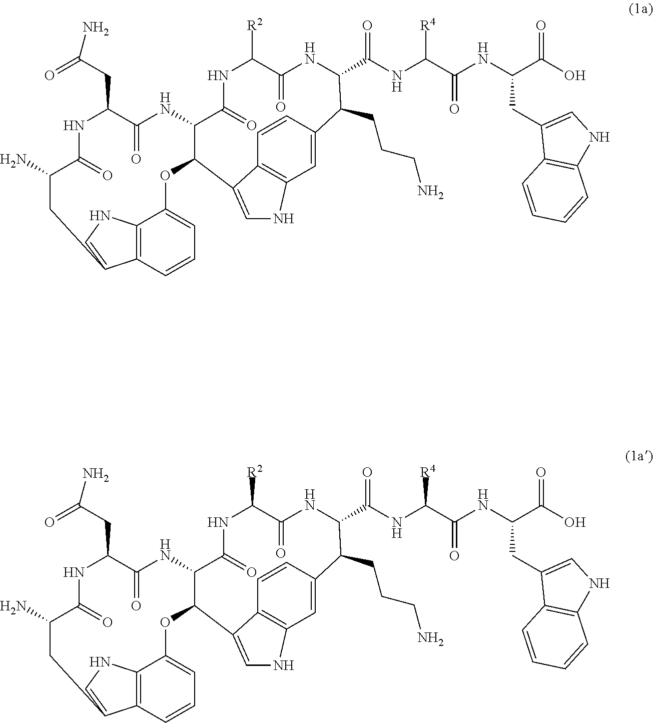

or a salt (especially a pharmaceutically acceptable salt) thereof, wherein R² and R⁴ are as defined above.

Compounds including suitable combinations of preferred embodiments of the compounds according to the invention, or a salt thereof, are particularly preferred; e.g. a compound or salt thereof including a combination of preferred embodiments of residues R¹, R², R³, R⁴ and R⁵ as disclosed herein. In other words, the present invention specifically encompasses all possible combinations of residues as indicated above, which result in a stable compound.

The most preferred compounds of the present invention are the compounds disclosed in the examples, or a salt thereof.

The expression alkyl refers to a saturated, straight-chain or branched hydrocarbon group that contains from 1 to 20 carbon atoms, preferably from 1 to 10 carbon atoms, especially from 1 to 6 (e.g. 1, 2, 3 or 4) carbon atoms, for example a methyl (Me, CH₃), ethyl (Et), n-propyl (nPr), iso-propyl (iPr), n-butyl (nBu), iso-butyl (iBu), sec-butyl (sBu), tert-butyl (tBu), n-pentyl, iso-pentyl, n-hexyl, 2,2-dimethylbutyl or n-octyl group.

Especially preferred alkyl groups are C₁₋₆ alkyl groups; moreover preferred alkyl groups are C₁₋₄ alkyl groups.

The expression C₁₋₆ alkyl refers to a saturated, straight-chain or branched hydrocarbon group that contains from 1 to 6 carbon atoms. The expression C₁₋₄ alkyl refers to a saturated, straight-chain or branched hydrocarbon group that contains from 1 to 4 carbon atoms. Examples are the methyl, ethyl, n-propyl, iso-propyl, n-butyl, iso-butyl, sec-butyl or the tert-butyl group.

The expressions alkenyl and alkynyl refer to at least partially unsaturated, straight-chain or branched hydrocarbon groups that contain from 2 to 20 carbon atoms, preferably from 2 to 10 carbon atoms, especially from 2 to 6 (e.g. 2, 3 or 4) carbon atoms, for example an ethenyl (vinyl), propenyl (allyl), iso-propenyl, butenyl, ethynyl (acetylenyl), propynyl (e.g. propargyl), butynyl, isoprenyl or hex-2-enyl group. Preferably, alkenyl groups have one or two (especially preferably one) double bond(s), and alkynyl groups have one or two (especially preferably one) triple bond(s).

Furthermore, the terms alkyl, alkenyl and alkynyl refer to groups in which one or more hydrogen atoms have been replaced by a halogen atom (preferably F or Cl) such as, for example, a 2,2,2-trichloroethyl or a trifluoromethyl group.

The expression heteroalkyl refers to an alkyl, alkenyl or alkynyl group in which one or more (preferably 1 to 8; especially preferably 1, 2, 3 or 4) carbon atoms have been replaced by an oxygen, nitrogen, phosphorus, boron, selenium, silicon or sulfur atom (preferably by an oxygen, sulfur or nitrogen atom) or by a SO or a SO₂ group. The expression heteroalkyl furthermore refers to a carboxylic acid or to a group derived from a carboxylic acid, such as, for example, acyl, acylalkyl, alkoxycarbonyl, acyloxy, acyloxyalkyl, carboxyalkylamide or alkoxycarbonyloxy. Furthermore, the term heteroalkyl refers to groups in which one or more hydrogen atoms have been replaced by a halogen atom (preferably F or Cl).

Preferably, a heteroalkyl group contains from 1 to 12 carbon atoms and from 1 to 8 heteroatoms selected from oxygen, nitrogen and sulfur (especially oxygen and nitrogen). Especially preferably, a heteroalkyl group contains from 1 to 6 (e.g. 1, 2, 3 or 4) carbon atoms and 1, 2, 3 or 4 (especially 1, 2 or 3) heteroatoms selected from oxygen, nitrogen and sulfur (especially oxygen and nitrogen). The term C₁-C₁₀ heteroalkyl refers to a heteroalkyl group containing from 1 to 10 carbon atoms and 1, 2, 3, 4, 5 or 6 heteroatoms selected from O, S and/or N (especially O and/or N). The term C₁-C₆ heteroalkyl refers to a heteroalkyl group containing from 1 to 6 carbon atoms and 1, 2, 3 or 4 heteroatoms selected from O, S and/or N (especially O and/or N). The term C₁-C₄ heteroalkyl refers to a heteroalkyl group containing from 1 to 4 carbon atoms and 1, 2 or 3 heteroatoms selected from O, S and/or N (especially O and/or N).

Further preferably, the expression heteroalkyl refers to an alkyl group as defined above (straight-chain or branched) in which one or more (preferably 1 to 6; especially preferably 1, 2, 3 or 4) carbon atoms have been replaced by an oxygen, sulfur or nitrogen atom or a CO group or a SO group or a SO₂ group; this group preferably contains from 1 to 6 (e.g. 1, 2, 3 or 4) carbon atoms and 1, 2, 3 or 4 (especially 1, 2 or 3) heteroatoms selected from oxygen, nitrogen and sulfur (especially oxygen and nitrogen); this group may preferably be substituted by one or more (preferably 1 to 6; especially preferably 1, 2, 3 or 4) fluorine, chlorine, bromine or iodine atoms or OH, ═O, SH, ═S, NH₂, ═NH, N₃, CN or NO₂ groups.

Examples of heteroalkyl groups are groups of formulae: R^{a}—O—Y^{a}—, R^{a}—S—Y^{a}—, R^{a}—SO—Y^{a}—R^{a}—SO₂—Y^{a}—, R^{a}—N(R^{b})—SO₂—Y^{a}—, R^{a}—SO₂—N(R^{b})—Y^{a}—, R^{a}—N(R^{b})—Y^{a}—, R^{a}—CO—Y^{a}—, R^{a}—O—CO—Y^{a}—, R^{a}—CO—O—Y^{a}—, R^{a}—CO—N(R^{b})—Y^{a}—, R^{a}—N(R^{b})—CO—Y^{a}—, R^{a}—O—CO—N(R^{b})—Y^{a}—, R^{a}—N(R^{b})—CO—O—Y^{a}—, R^{a}—N(R^{b})—CO—N(R^{c})—Y^{a}—, R^{a}—O—CO—O—Y^{a}—, R^{a}—N(R^{b})—C(═NR^{d})—N(R^{c})—Y^{a}—, R^{a}—CS—Y^{a}—, R^{a}—O—CS—Y^{a}—, R^{a}—CS—O—Y^{a}—, R^{a}—CS—N(R^{b})—Y^{a}—, R^{a}—N(R^{b})—CS—Y^{a}—, R^{a}—O—CS—N(R^{b})—Y^{a}—, R^{a}—N(R^{b})—CS—O—Y^{a}—, R^{a}—N(R^{b})—CS—N(R^{c})—Y^{a}—, R^{a}—O—CS—O—Y^{a}—, R^{a}—S—CO—Y^{a}—, R^{a}—CO—S—Y^{a}—, R^{a}—S—CO—N(R^{b})—Y^{a}—, R^{a}—N(R^{b})—CO—S—Y^{a}—, R^{a}—S—CO—O—Y^{a}—, R^{a}—O—CO—S—Y^{a}—, R^{a}—S—CO—S—Y^{a}—, R^{a}—S—CS—Y^{a}—, R^{a}—CS—S—Y^{a}—R^{a}—S—CS—N(R^{b})—Y^{a}—, R^{a}—N(R^{b})—CS—S—Y^{a}—, R^{a}—S—CS—O—Y^{a}—, R^{a}—O—CS—S—Y^{a}—, wherein R^{a} being a hydrogen atom, a C₁-C₆ alkyl, a C₂-C₆ alkenyl or a C₂-C₆ alkynyl group; R^{b} being a hydrogen atom, a C₁-C₆ alkyl, a C₂-C₆ alkenyl or a C₂-C₆ alkynyl group; R^{c} being a hydrogen atom, a C₁-C₆ alkyl, a C₂-C₆ alkenyl or a C₂-C₆ alkynyl group; R^{d} being a hydrogen atom, a C₁-C₆ alkyl, a C₂-C₆ alkenyl or a C₂-C₆ alkynyl group and Y^{a} being a bond, a C₁-C₆ alkylene, a C₂-C₆ alkenylene or a C₂-C₆ alkynylene group, wherein each heteroalkyl group contains at least one carbon atom and one or more hydrogen atoms may be replaced by fluorine or chlorine atoms.

Specific examples of heteroalkyl groups are methoxy, trifluoromethoxy, ethoxy, n-propyloxy, iso-propyloxy, n-butoxy, tert-butyloxy, methoxymethyl, ethoxymethyl, —CH₂CH₂OH, —CH₂OH, —SO₂Me, —NHAc, methoxyethyl, 1-methoxyethyl, 1-ethoxyethyl, 2-methoxyethyl or 2-ethoxyethyl, methylamino, ethylamino, propylamino, isopropylamino, dimethylamino, diethylamino, isopropylethylamino, methylamino methyl, ethylamino methyl, diisopropylamino ethyl, methylthio, ethylthio, isopropylthio, enol ether, dimethylamino methyl, dimethylamino ethyl, acetyl, propionyl, butyryloxy, acetyloxy, methoxycarbonyl, ethoxycarbonyl, propionyloxy, acetylamino or propionylamino, carboxymethyl, carboxyethyl or carboxypropyl, N-ethyl-N-methylcarbamoyl or N-methyl-carbamoyl. Further examples of heteroalkyl groups are nitrile (—CN), isonitrile, cyanate, thiocyanate, isocyanate, isothiocyanate and alkylnitrile groups.

The expression cycloalkyl refers to a saturated or partially unsaturated (for example, a cycloalkenyl group) cyclic group that contains one or more rings (preferably 1 or 2), and contains from 3 to 14 ring carbon atoms, preferably from 3 to 10 (especially 3, 4, 5, 6 or 7) ring carbon atoms. The expression cycloalkyl refers furthermore to groups in which one or more hydrogen atoms have been replaced by fluorine, chlorine, bromine or iodine atoms or by OH, ═O, SH, ═S, NH₂, ═NH, N₃ or NO₂ groups, thus, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetraline, cyclopentylcyclohexyl, fluorocyclohexyl or cyclohex-2-enyl group. Preferably, the expression cycloalkyl refers to a saturated cyclic group that contains one or more rings (preferably 1 or 2), and contains from 3 to 14 ring carbon atoms, preferably from 3 to 10 (especially 3, 4, 5, 6 or 7) ring carbon atoms.

The expression heterocycloalkyl refers to a cycloalkyl group as defined above in which one or more (preferably 1, 2 or 3) ring carbon atoms have been replaced by an oxygen, nitrogen, silicon, selenium, phosphorus or sulfur atom (preferably by an oxygen, sulfur or nitrogen atom) or a SO group or a SO₂ group. A heterocycloalkyl group has preferably 1 or 2 ring(s) and 3 to 10 (especially 3, 4, 5, 6 or 7) ring atoms (preferably selected from C, O, N and S). The expression heterocycloalkyl refers furthermore to groups that are substituted by fluorine, chlorine, bromine or iodine atoms or by OH, ═O, SH, ═S, NH₂, ═NH, N₃ or NO₂ groups. Examples are a piperidyl, prolinyl, imidazolidinyl, piperazinyl, morpholinyl (e.g. —N(CH₂CH₂)₂O), urotropinyl, pyrrolidinyl, tetrahydrothiophenyl, tetrahydropyranyl, tetrahydrofuryl or 2-pyrazolinyl group and also lactames, lactones, cyclic imides and cyclic anhydrides.

The expression alkylcycloalkyl refers to groups that contain both cycloalkyl and alkyl, alkenyl or alkynyl groups in accordance with the above definitions, for example alkylcycloalkyl, cycloalkylalkyl, alkylcycloalkenyl, alkenylcycloalkyl and alkynylcycloalkyl groups. An alkylcycloalkyl group preferably contains a cycloalkyl group that contains one or two rings and from 3 to 10 (especially 3, 4, 5, 6 or 7) ring carbon atoms, and one or two alkyl, alkenyl or alkynyl groups (especially alkyl groups) having 1 or 2 to 6 carbon atoms.

The expression heteroalkylcycloalkyl refers to alkylcycloalkyl groups as defined above in which one or more (preferably 1, 2 or 3) carbon atoms have been replaced by an oxygen, nitrogen, silicon, selenium, phosphorus or sulfur atom (preferably by an oxygen, sulfur or nitrogen atom) or a SO group or a SO₂ group. A heteroalkylcycloalkyl group preferably contains 1 or 2 rings having from 3 to 10 (especially 3, 4, 5, 6 or 7) ring atoms, and one or two alkyl, alkenyl, alkynyl or heteroalkyl groups (especially alkyl or heteroalkyl groups) having from 1 or 2 to 6 carbon atoms. Examples of such groups are alkylheterocycloalkyl, alkylheterocycloalkenyl, alkenylheterocycloalkyl, alkynylheterocycloalkyl, heteroalkylcycloalkyl, heteroalkylheterocycloalkyl and heteroalkylheterocycloalkenyl, the cyclic groups being saturated or mono-, di- or tri-unsaturated.

The expression aryl refers to an aromatic group that contains one or more rings and from 6 to 14 ring carbon atoms, preferably from 6 to 10 (especially 6) ring carbon atoms. The expression aryl refers furthermore to groups that are substituted by fluorine, chlorine, bromine or iodine atoms or by OH, SH, NH₂, N₃ or NO₂ groups. Examples are the phenyl (Ph), naphthyl, biphenyl, 2-fluorophenyl, anilinyl, 3-nitrophenyl or 4-hydroxyphenyl group.

The expression heteroaryl refers to an aromatic group that contains one or more rings and from 5 to 14 ring atoms, preferably from 5 to 10 (especially 5 or 6 or 9 or 10) ring atoms, comprising one or more (preferably 1, 2, 3 or 4) oxygen, nitrogen, phosphorus or sulfur ring atoms (preferably O, S or N). The expression heteroaryl refers furthermore to groups that are substituted by fluorine, chlorine, bromine or iodine atoms or by OH, SH, N₃, NH₂ or NO₂ groups. Examples are pyridyl (e.g. 4-pyridyl), imidazolyl (e.g. 2-imidazolyl), phenylpyrrolyl (e.g. 3-phenylpyrrolyl), thiazolyl, isothiazolyl, 1,2,3-triazolyl, 1,2,4-triazolyl, oxadiazolyl, thiadiazolyl, indolyl, indazolyl, tetrazolyl, pyrazinyl, pyrimidinyl, pyridazinyl, 4-hydroxypyridyl (4-pyridonyl), 3,4-hydroxypyridyl (3,4-pyridonyl), oxazolyl, isoxazolyl, triazolyl, tetrazolyl, isoxazolyl, indazolyl, indolyl, benzimidazolyl, benzoxazolyl, benzisoxazolyl, benzthiazolyl, pyridazinyl, quinolinyl, isoquinolinyl, pyrrolyl, purinyl, carbazolyl, acridinyl, pyrimidyl, 2,3′-bifuryl, pyrazolyl (e.g. 3-pyrazolyl) and isoquinolinyl groups.

The expression aralkyl refers to groups containing both aryl and also alkyl, alkenyl, alkynyl and/or cycloalkyl groups in accordance with the above definitions, such as, for example, arylalkyl, arylalkenyl, arylalkynyl, arylcycloalkyl, arylcycloalkenyl, alkylarylcycloalkyl and alkylarylcycloalkenyl groups. Specific examples of aralkyls are phenylcyclopentyl, cyclohexylphenyl as well as groups derived from toluene, xylene, mesitylene, styrene, benzyl chloride, o-fluorotoluene, 1H-indene, tetraline, dihydronaphthalene, indanone, cumene, fluorene and indane. An aralkyl group preferably contains one or two aromatic ring systems (especially 1 or 2 rings), each containing from 6 to 10 carbon atoms and one or two alkyl, alkenyl and/or alkynyl groups containing from 1 or 2 to 6 carbon atoms and/or a cycloalkyl group containing 3, 4, 5, 6 or 7 ring carbon atoms.

The expression heteroaralkyl refers to groups containing both aryl and/or heteroaryl groups and also alkyl, alkenyl, alkynyl and/or heteroalkyl and/or cycloalkyl and/or het-erocycloalkyl groups in accordance with the above definitions. A heteroaralkyl group preferably contains one or two aromatic ring systems (especially 1 or 2 rings), each containing from 5 or 6 to 9 or 10 ring atoms (preferably selected from C, N, O and S) and one or two alkyl, alkenyl and/or alkynyl groups containing 1 or 2 to 6 carbon atoms and/or one or two heteroalkyl groups containing 1 to 6 carbon atoms and 1, 2 or 3 heteroatoms selected from O, S and N and/or one or two cycloalkyl groups each containing 3, 4, 5, 6 or 7 ring carbon atoms and/or one or two heterocycloalkyl groups, each containing 3, 4, 5, 6 or 7 ring atoms comprising 1, 2, 3 or 4 oxygen, sulfur or nitrogen atoms.

Examples are arylheteroalkyl, arylheterocycloalkyl, arylheterocycloalkenyl, arylalkylheterocycloalkyl, arylalkenylheterocycloalkyl, arylalkynylheterocycloalkyl, arylalkylheterocycloalkenyl, heteroarylalkyl, heteroarylalkenyl, heteroarylalkynyl, heteroarylheteroalkyl, heteroarylcycloalkyl, heteroarylcycloalkenyl, heteroaryl-heterocycloalkyl, heteroarylheterocycloalkenyl, heteroarylalkylcycloalkyl, heteroaryl-alkylheterocycloalkenyl, heteroarylheteroalkylcycloalkyl, heteroarylheteroalkyl-cycloalkenyl and heteroarylheteroalkylheterocycloalkyl groups, the cyclic groups being saturated or mono-, di- or tri-unsaturated. Specific examples are a tetrahydroisoquinolinyl, benzoyl, phthalidyl, 2- or 3-ethylindolyl, 4-methylpyridino, 2-, 3- or 4-methoxyphenyl, 4-ethoxyphenyl, 2-, 3- or 4-carboxyphenylalkyl group.

As already stated above, the expressions cycloalkyl, heterocycloalkyl, alkylcycloalkyl, heteroalkylcycloalkyl, aryl, heteroaryl, aralkyl and heteroaralkyl also refer to groups that are substituted by fluorine, chlorine, bromine or iodine atoms or by OH, ═O, SH, ═S, NH₂, ═NH, N₃ or NO₂ groups.

The term halogen refers to F, Cl, Br or I. Preferred halogens are F, Cl and Br. Especially preferred halogens are F and Cl.

The term “optionally substituted” refers to a group, which is unsubstituted or substituted by one or more (especially by one, two or three; preferably by one or two) substituents.

If a group comprises more than one substituent, these substituents are independently selected, i.e., they may be the same or different.

Examples for substituents are fluorine, chlorine, bromine and iodine and OH, SH, NH₂, —SO₃H, —SO₂NH₂, —COOH, —COOMe, —COMe (Ac), —NHSO₂Me, —SO₂NMe₂, —CH₂NH₂, —NHAc, —SO₂Me, —CONH₂, —CN, —NHCONH₂, —NHC(NH)NH₂, —NOHCH₃, —N₃ and —NO₂ groups. Further examples of substituents are C₁-C₁₀ alkyl, C₂-C₁a alkenyl, C₂-C₁a alkynyl, C₁-C₁₀ heteroalkyl, C₃-C₁₈ cycloalkyl, C₁-C₁₇ heterocycloalkyl, C₄-C₂₀ alkylcycloalkyl, C₁-C₁₉ heteroalkylcycloalkyl, C₆-C₁₈ aryl, C₁-C₁₇ heteroaryl, C₇-C₂₀ aralkyl and C₁-C₁₉ heteroaralkyl groups; especially C₁-C₆ alkyl, C₂-C₆ alkenyl, C₂-C₆ alkynyl, C₁-C₆ heteroalkyl, C₃-C₁₀ cycloalkyl, C₁-C₉ heterocycloalkyl, C₄-C₁₂ alkylcycloalkyl, C₁-C₁₁ heteroalkylcycloalkyl, C₆-C₁₀ aryl, C₁-C₉ heteroaryl, C₇-C₁₂ aralkyl and C₁-C₁₁ heteroaralkyl groups, further preferably C₁-C₆ alkyl and C₁-C₆ heteroalkyl groups.

When an aryl, heteroaryl, cycloalkyl, alkylcycloalkyl, heteroalkylcycloalkyl, heterocycloalkyl, aralkyl or heteroaralkyl group contains more than one ring, these rings may be bonded to each other via a single or double bond or these rings may be annulated or fused or bridged.

Owing to their substitution, the compounds of the present invention may contain one or more centers of chirality. The present invention therefore includes both all pure enantiomers and all pure diastereomers and also mixtures thereof in any mixing ratio. The present invention moreover also includes all cis/trans-isomers of the compounds of the present invention and also mixtures thereof. The present invention moreover includes all tautomeric forms of the compounds of the present invention.

The present invention further provides pharmaceutical compositions comprising a compound or a salt thereof according to the present invention and optionally one or more carrier substances and/or one or more adjuvants and/or one or more further active pharmaceutical ingredient(s).

The present invention furthermore provides compounds (or a salt thereof) or pharmaceutical compositions as described herein for use as a medicament, e.g. for use in the prophylaxis or treatment of a bacterial infection; especially for use in the prophylaxis or treatment of a bacterial infection caused by Gram-negative bacteria.

The compounds and/or compositions described herein can also be useful in the prophylaxis treatment of infections by bacteria, which are susceptible or multidrug resistant, polymyxin resistant mutant, carbapenam-resistant bacteria, methicillin-resistant Staphylococcus aureus, vancomycin-resistant Enterococci or multi-drug resistant Neisseria gonorrhoeae.

Examples of Gram-negative bacteria include, but are not limited to, Escherichia coli, Pseudomonas aeruginosa, Candidatus liberibacter, Agrobacterium tumefaciens, Branhamella catarrhalis, Citrobacter di versus, Enterobacter aerogenes, Klebsiella pneumoniae, Proteus mirabilis, Salmonella typhimurium, Neisseria meningitidis, Serratia marcescens, Shigella sonnei, Shigella boydii, Neisseria gonorrhoeae, Acinetobacter baumannii, Salmonella enteriditis, Fusobacterium nucleatum, Veillonella parvula, Actinobacillus actinomycetemcomitans, Aggregatibacter actinomycetemcomitans, Porphyromonas gingivalis, Helicobacter pylon, Francisella tularensis, Yersinia pestis, Vibrio cholera, Morganella morganii, Ectwardsiella tarda, Campylobacter jejuni, or Haemophilus influenza, Enterobacter cloacae and numerous others. Other notable groups of Gram-negative bacteria include the cyanobacteria, spirochaetes, green sulfur and green non-sulfur bacteria.

The present invention further provides a compound (or a salt thereof) as described herein or a pharmaceutical composition as defined herein for the preparation of a medicament for use in the prophylaxis or treatment of a bacterial infection; especially for use in the prophylaxis or treatment of a bacterial infection caused by Gram-negative bacteria.

Examples of salts of sufficiently basic compounds are salts of physiologically acceptable mineral acids like hydrochloric, hydrobromic, sulfuric and phosphoric acid; or salts of organic acids like methanesulfonic, p-toluenesulfonic, lactic, acetic, trifluoroacetic, citric, succinic, fumaric, maleic and salicylic acid. Further, a sufficiently acidic compound may form alkali or earth alkali metal salts, for example sodium, potassium, lithium, calcium or magnesium salts; ammonium salts; or organic base salts, for example methylamine, dimethylamine, trimethylamine, triethylamine, ethylenediamine, ethanolamine, choline hydroxide, meglumin, piperidine, morpholine, tris-(2-hydroxyethyl)amine, lysine or arginine salts; all of which are also further examples of salts of the compounds described herein.

The compounds described herein may be solvated, especially hydrated. The solvation/hydration may occur during the process of production or as a consequence of the hygroscopic nature of the initially water-free compounds. The solvates and/or hydrates may e.g. be present in solid or liquid form.

The therapeutic use of the compounds described herein, their salts, as well as formulations and pharmaceutical compositions also lie within the scope of the present invention.

In general, the compounds and pharmaceutical compositions described herein will be administered by using the established and acceptable modes known in the art.

For oral administration, such therapeutically useful agents can be administered by one of the following routes: oral, e.g. as tablets, dragees, coated tablets, pills, semisolids, soft or hard capsules, for example soft and hard gelatine capsules, aqueous or oily solutions, emulsions, suspensions or syrups, parenteral including intravenous, intramuscular and subcutaneous injection, e.g. as an injectable solution or suspension, rectal as suppositories, by inhalation or insufflation, e.g. as a powder formulation, as microcrystals or as a spray (e.g. liquid aerosol), transdermal, for example via an transdermal drug delivery system (TDDS) such as a plaster containing the active ingredient or intranasal. For the production of such tablets, pills, semisolids, coated tablets, dragees and hard, e.g. gelatine, capsules the therapeutically useful product may be mixed with pharmaceutically inert, inorganic or organic excipients as are e.g. lactose, sucrose, glucose, gelatine, malt, silica gel, starch or derivatives thereof, talc, stearinic acid or their salts, dried skim milk, and the like. For the production of soft capsules, one may use excipients as are e.g. vegetable, petroleum, animal or synthetic oils, wax, fat, and polyols. For the production of liquid solutions, emulsions or suspensions or syrups one may use as excipients e.g. water, alcohols, aqueous saline, aqueous dextrose, polyols, glycerin, lipids, phospholipids, cyclodextrins, vegetable, petroleum, animal or synthetic oils. Especially preferred are lipids and more preferred are phospholipids (preferred of natural origin, especially preferred with a particle size between 300 to 350 nm) preferred in phosphate buffered saline (pH=7 to 8, preferred 7.4). For suppositories, one may use excipients as are e.g. vegetable, petroleum, animal or synthetic oils, wax, fat and polyols. For aerosol formulations, one may use compressed gases suitable for this purpose, e.g. oxygen, nitrogen and carbon dioxide. The pharmaceutically useful agents may also contain additives for conservation, stabilization, e.g. UV stabilizers, emulsifiers, sweetener, aromatizers, salts to change the osmotic pressure, buffers, coating additives and antioxidants.

In general, in the case of oral or parenteral administration to adult humans weighing approximately 80 kg, a daily dosage of about 1 mg to about 10,000 mg, preferably from about 5 mg to about 1,000 mg, should be appropriate, although the upper limit may be exceeded when indicated. The daily dosage can be administered as a single dose or in divided doses, or for parenteral administration, it may be given as continuous infusion or subcutaneous injection.

According to a moreover preferred embodiment, the present invention provides a method for treating a bacterial infection, which comprises administering to a subject in need of such treatment a therapeutically effective amount of a compound as described herein, or a salt thereof.

According to a further preferred embodiment, the present invention provides a method of treating, ameliorating, or preventing a bacterial infection in a subject in need thereof, comprising administering to the subject a therapeutically effective amount of at least one compound described herein, a salt thereof, or a pharmaceutical composition comprising at least one of the (specific) compounds, or a salt thereof, described herein. Administration of the compound may be topical, such as subcutaneous, transdermal, rectal, intravaginal, intranasal, intrabronchial, intraocular, or intra-aural. Alternatively, administration may be systemic, such as oral administration In still other alternatives, administration may be parenteral, intravenous, intramuscular, or intraperitoneal.

As used herein, the term “administration” can also include administering a combination of compounds. Thus, administration may be in the form of dosing an organism with a compound or combination of compounds, such that the organism's circulatory system will deliver a compound or combination of compounds to the target area, including but not limited to a cell or cells, synaptic junctions and circulation. Administration may also mean that a compound or combination of compounds is placed in direct contact with an organ, tissue, area, region, cell or group of cells, such as but not limited to direct injection of the combination of compounds.

In select embodiments, a combination of compounds can be administered, and thus the individual compounds can also be said to be co-administered with one another. As used herein, “co-administer” indicates that each of at least two compounds is administered during a time frame wherein the respective periods of biological activity or effects overlap. Thus, the term co-administer includes sequential as well as coextensive administration of the individual compounds, at least one of which is a compound of the present invention. Accordingly, “administering” a combination of compounds according to some of the methods of the present invention includes sequential as well as coextensive administration of the individual compounds of the present invention. Likewise, the phrase “combination of compounds” indicates that the individual compounds are co-administered, and the phrase “combination of compounds” does not mean that the compounds must necessarily be administered contemporaneously or coextensively. In addition, the routes of administration of the individual compounds need not be the same.

The invention also relates to a combination preparation or pharmaceutical composition containing at least one compound according to the invention, or a salt thereof, and at least one further (different) active pharmaceutical ingredient. The combination preparation of the invention can be used as a medicament, in particular in the treatment or prophylaxis of a bacterial infection with Gram-negative bacteria or Gram-negative and -positive bacteria.

Preferably, in the combination preparation or pharmaceutical composition of the invention the further active pharmaceutical ingredient is another antibiotic. The other antibiotic can be selected from the group consisting of β-lactam antibiotics, including penams, carbapenams, oxapenams, penems, carbapenems, monobactams, cephems, carbacephems, oxacephems, and monobactams; aminoglycoside antibiotics, including amikacin, arbekacin, astromicin, bekanamycin, dibekacin, framycetin, gentamicin, hygromycin B, isepamicin, kanamycin, neomycin, netilmicin, paromomycin, paromomycin sulfate, ribostamycin, sisomicin, spectinomycin, streptomycin, tobramycin, and verdamicin; quinolone antibiotics, including ciprofloxacin, enoxacin, gatifloxacin, grepafloxacin, levofloxacin, lomefloxacin, moxifloxacin, nalidixic acid, norfloxacin, ofloxacin, sparfloxacin, temafloxacin, and trovafloxacin; or glycopeptide antibiotics, e.g. vancomycin, telavancin, bleomycin, ramoplanin, and decaplanin; linezolid; or daptomycin.

As used herein, the terms “treat” and “treatment” refer to a slowing of or a reversal of the progress of the disease or infection. Treating a disease includes treating a symptom and/or reducing the symptoms of the disease or infection. The terms “prophylaxis” and “preventing” refer to a slowing of the disease or of the onset of the disease, infection or the symptoms thereof. Prophylaxis of, or preventing, a disease or infection can include stopping the onset of the disease, infection or symptom thereof.

As used herein, the term “subject” may be an animal, vertebrate animal, mammal, rodent (e.g., a guinea pig, a hamster, a rat, a mouse), a murine (e g., a mouse), a canine (e g., a dog), a feline (e.g. a cat), an equine (e.g., a horse), a primate, a simian (e.g., a monkey or ape), a monkey (e.g., marmoset, a baboon), an ape (e.g., gorilla, chimpanzee, orangutan, gibbon), or a human.

As used herein, the term “dosage unit” refers to a physically discrete unit, such as a capsule or tablet suitable as a unitary dosage for a subject. Each unit contains a predetermined quantity of a compound of the invention, or a salt thereof, which was discovered or believed to produce the desired pharmacokinetic profile, which yields the desired therapeutic effect. The dosage unit is composed of a compound of the present invention, or a salt thereof, in association with at least one pharmaceutically acceptable carrier, salt, excipient or a combination thereof. The term “dose” or “dosage” refers to the amount of active ingredient that an individual takes or is administered at one time.

The term “therapeutically effective amount” refers to the amount sufficient to produce a desired biological effect in a subject. Accordingly, a therapeutically effective amount of a compound may be an amount which is sufficient to treat or prevent a disease or infection, and/or delay the onset or progression of a disease or infection, and or alleviate one or more symptoms of the disease or infection, when administered to a subject suffered from or susceptible to that disease or infection. A “pharmaceutically acceptable carrier” or “pharmaceutically acceptable excipient” herein refers to a non-API (where API refers to Active Pharmaceutical Ingredient) substances such as disintegrators, binders, fillers, and lubricants used in formulating pharmaceutical products. They are generally safe for administering to humans.

The term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, and the like.

In a further aspect, the present invention provides a method for the production of a compound of formula (Ic), or a compound of formula (Ic′) or (Ic″):

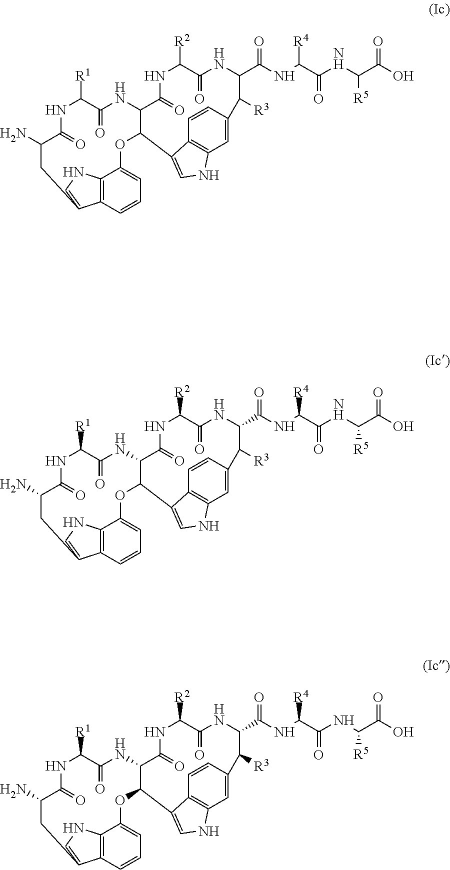

wherein R¹, R², R⁴ and R⁵ each independently represents H, CH₃, CH₂OH or a group:

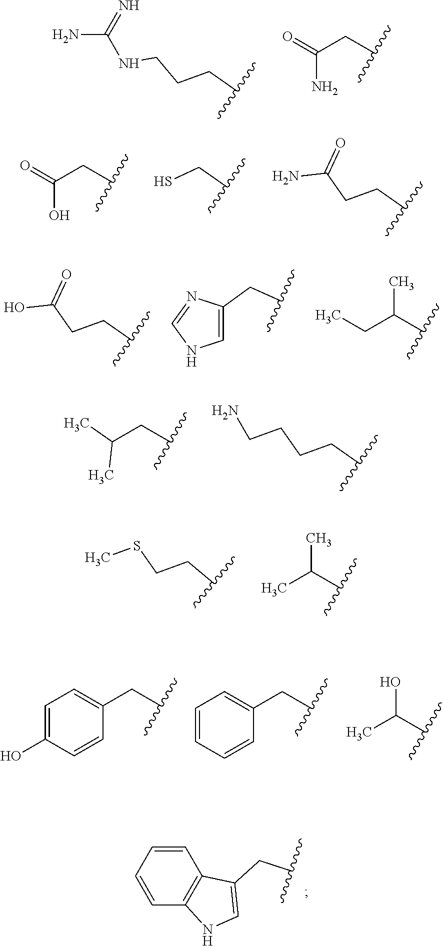

and
R³ represents H, OH, SH, COOH, CONH₂ or a group:

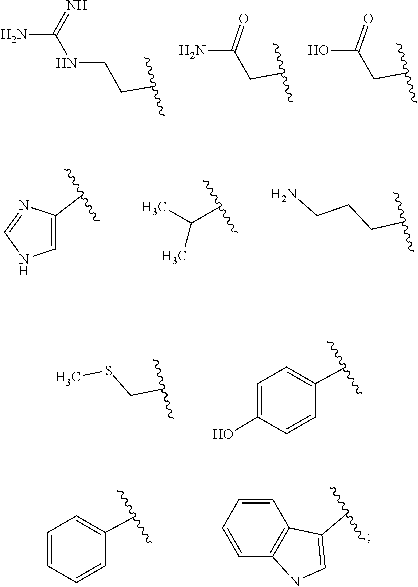

wherein the method comprises the steps of:
(a) culturing a bacterial host cell comprising:
(aa) at least one synthetic or recombinant nucleic acid sequence, which encodes a biosynthetic gene cluster of a compound of formula (Ic) (or (Ic′), or (Ic″)) and has:
(i) a sequence identity to the full-length sequence of SEQ ID NO. 1 of at least 95%, 96%, 97%, 98%, 98.5%, 99%, or 99.5% to 100%; or
(ii) a sequence completely complementary to any nucleic acid sequence of (i); or
(ab) a plasmid comprising at least one synthetic or recombinant nucleic acid sequence as defined in (aa); and
(b) separating and retaining the compound of formula (Ic) (or (Ic′), or (Ic″)) from the culture broth.

In a production method of the invention, the compound of the invention can be prepared by growing a recombinant host, such as a microorganism, e.g., a bacterium such as a bacterium of the species of E. coli, Bacillus, Corynebacteria (e.g. C. glutamicum), Lactobacillus (e.g. Lactococcus lactis) or Streptomyces (e.g. S. albus, S. lividans), or a yeast such as Saccharomyces cerevisiae, Saccharomyces pombe, Pichia pastoris or Yarrowia lipolytica, under controlled conditions (e.g., as known in the art or described hereinafter), and recovering the compound from the culture broth in substantially pure form as described herein. In the production method of the invention, the compound is prepared by growing the respective bacterial host cell under controlled conditions as described hereinafter, and recovering the compound from the culture broth in substantially pure form as described herein.

The culturing step can, for example, be performed in liquid culture, by growing the respective recombinant host (e.g., microorganism, e.g. bacterial host cell) in media containing one or several different carbon sources, and one or different nitrogen sources. Also salts are essential for growth and production. Suitable carbon sources are different mono-, di-, and polysaccharides like maltose, glucose or carbon from amino acids like peptones. Nitrogen sources are ammonium, nitrate, urea, chitin or nitrogen from amino acids. The following inorganic ions support the growth or are essential in synthetic media: Mg-ions, Ca-ions, Fe-ions, Mn-ions, Zn-ions, K-ions, sulfate-ions, CI-ions, phosphate-ions. Preferred compositions of nutrient media used in the production method of the invention are described in more detail in the examples. In an embodiment, the recombinant host is a microorganism, e.g., a bacterium such as a bacterium of the species of E. coli, Bacillus, Corynebacteria (e.g. C. glutamicum), Lactobacillus (e.g. Lactococcus lactis) or Streptomyces (e.g. S. albus, S. lividans), or a yeast such as Saccharomyces cerevisiae, Saccharomyces pombe, Pichia pastoris or Yarrowia lipolytica. In one embodiment, the recombinant host can be a bacterial host cell, e.g. an Escherichia coli or a Lactobacillus cell, preferably an E. coli cell, and more preferably an E. coli BL21(DE3) cell. Preferred embodiments include E. coli BL21 (DE3) pNOSOdarA-E-D9 (deposited at DSMZ on 29 Jan. 2021; DSM 33798) and E. coli BL21 (DE3) pNOSOdarA-E-D17 (deposited at DSMZ on 29 Jan. 2021; DSM 33799). Temperatures for growth and production are between 15° C. to 40° C., preferred temperatures are between 25° C. and 35° C., especially at 30° C. The pH of the culture solution is from 5 to 8, preferably a pH of 6.9 to 7.2, more preferable a pH of about 7.1.

The compound may be recovered from the fermentation broth by resin absorption and eluted from the resin by washing with solvents of various polarities. Purification may be furthered by chromatographic separation such as high performance liquid chromatography (HPLC) or reverse-phase high performance liquid chromatography (RP-HPLC).

Compounds of formula (Ic), (Ic′) or (Ic″), i.e. darobactin A and darobactin derivatives, are ribosomally synthesized and post-translationally modified peptide compounds (RiPPs). The artificial synthetic or recombinant biosynthetic gene cluster (BGC) encoding said peptide compounds comprises (consists of) genes with the following predicted functions: darA or modified variant thereof: darobactin propeptide or propetide of darobactin derivative; darBCD: ABC-type trans envelope exporter; and darE: radical S-adenosyl methionine (SAM) methyltransferase (proposed to catalyze the cyclization reactions to connect W₃-K₅ and W₁-W₃). The bacterial host cell comprises at least one synthetic or recombinant nucleic acid sequence, which encodes such a synthetic or recombinant BGC of a compound of formula (Ic), (Ic′) or (Ic″), and thus being capable of synthesizing a compound according to formula (Ic), (Ic′) or (Ic″). The synthetic or recombinant nucleic acid sequence utilized in the production method of the present invention, may, in addition to the nucleic acid sequence encoding the BGC, also include regulatory sequences, such as promoter and translation initiation and termination sequences, and can further include sequences that facilitate stable maintenance in a host cell, i.e., sequences that provide the function of an origin of replication or facilitate integration into host cell chromosomal or other DNA by homologous recombination. The synthetic or recombinant nucleic acid sequence utilized in the production method of the present invention is a synthetic or recombinant nucleic acid sequence, which encodes a BGC of a compound of formula (Ic) (or of formula (Ic′), or of formula (Ic″)), and has:
(i) a sequence identity to the full-length sequence of SEQ ID NO. 1 of at least 95%, 96%, 97%, 98%, 98.5%, 99%, or 99.5% to 100%, or
(ii) a sequence completely complementary to any nucleic acid sequence of (i).

The compounds of the invention described herein, i.e. darobactin derivatives, are ribosomally synthesized and post-translationally modified peptide compounds (RiPPs).

These peptide compounds can be produced using any method. For example, the compounds can be produced by chemical synthesis. Alternatively, peptide compounds described herein can be produced by standard recombinant technology using heterologous expression vectors encoding polypeptides. Expression vectors can be introduced into host cells, e.g., by transformation or transfection, for expression of the encoded polypeptide, which then can be purified. Expression systems that can be used for small or large scale production of polypeptides include, without limitation, microorganisms such as bacteria (e.g., species of E. coli, Bacillus, Corynebacteria (e.g. C. glutamicum), Lactobacillus (e.g. Lactococcus lactis) or Streptomyces (e.g. S. albus, S. lividans) transformed with recombinant bacteriophage DNA, plasmid DNA, or cosmid DNA expression vectors containing the nucleic acid molecules described herein, and yeast (e.g., Saccharomyces cerevisiae, Saccharomyces pombe, Pichia pastoris or Yarrowia lipolytica) transformed with recombinant yeast expression vectors containing the nucleic acid molecules described herein. Useful expression systems also include insect cell systems infected with recombinant virus expression vectors (e.g., baculovirus) containing the nucleic acid molecules described herein, and plant cell systems infected with recombinant virus expression vectors (e.g., tobacco mosaic virus) or transformed with recombinant plasmid expression vectors (e.g., Ti plasmid) containing the nucleic acid molecules described herein. Peptide compounds of this invention also can be produced using mammalian expression system harboring recombinant expression constructs containing promoters derived from the genome of mammalian cells (e.g., the metallothionein promoter) or from mammalian viruses (e.g., the adenovirus late promoter and the cytomegalovirus promoter), along with the nucleic acids described herein. Peptide compounds of this invention can have an N-terminal or C-terminal tag.

In one embodiment, described herein is a method for the production of a compound of formula (1′), (1b) or (1b′):

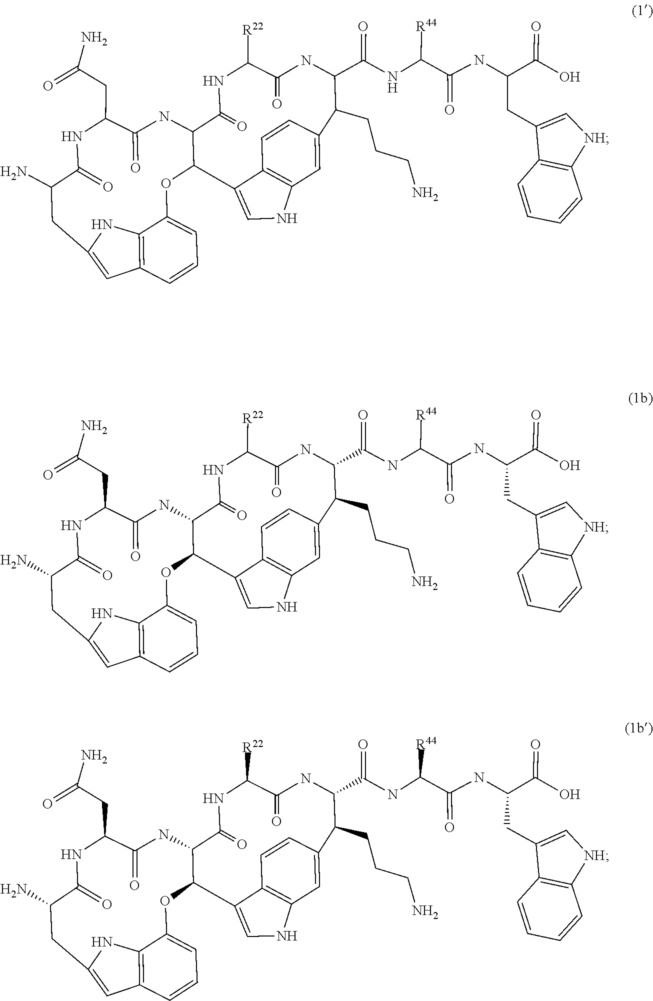

wherein each of R²² and R⁴⁴ independently of one another, represents H, CH₃, CH₂OH or a group:

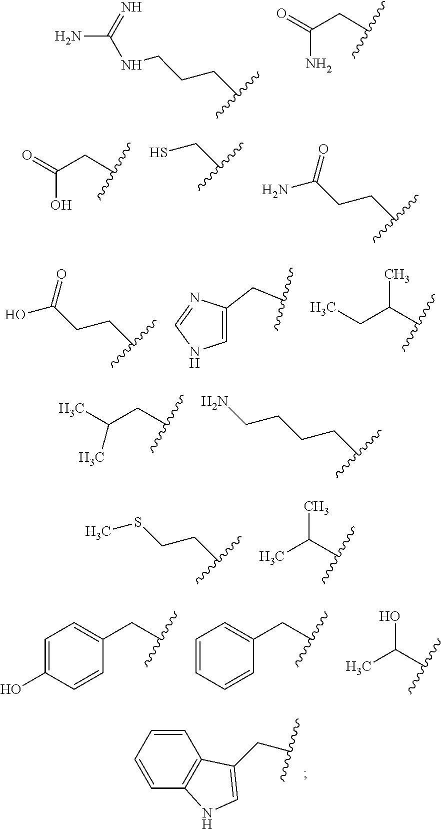

wherein the method comprises the steps of:
(a) providing a recombinant host capable of producing said compound of formula (1′), (1b) or (1b′), wherein said recombinant host harbors at least one synthetic or recombinant nucleic acid encoding a biosynthetic gene cluster (BGC) of the compound, which BGC has:
   (i) a sequence identity to the full-length sequence of SEQ ID NO. 44 of at least 95%, 96%, 97%, 98%, 98.5%, 99%, or 99.5% to 100%; or
   (ii) a sequence completely complementary to any nucleic acid sequence of (i);
(b) cultivating said recombinant host for a time sufficient for said recombinant host to produce the compound of formula (1′), (1b) or (1b′);
(c) isolating the compound from said recombinant host or from cultivation supernatant, thereby producing the compound of formula (1′), (1b) or (1b′).

In one embodiment, described herein is a method for the production of a compound of formula (II), (IIa) or (IIa′):

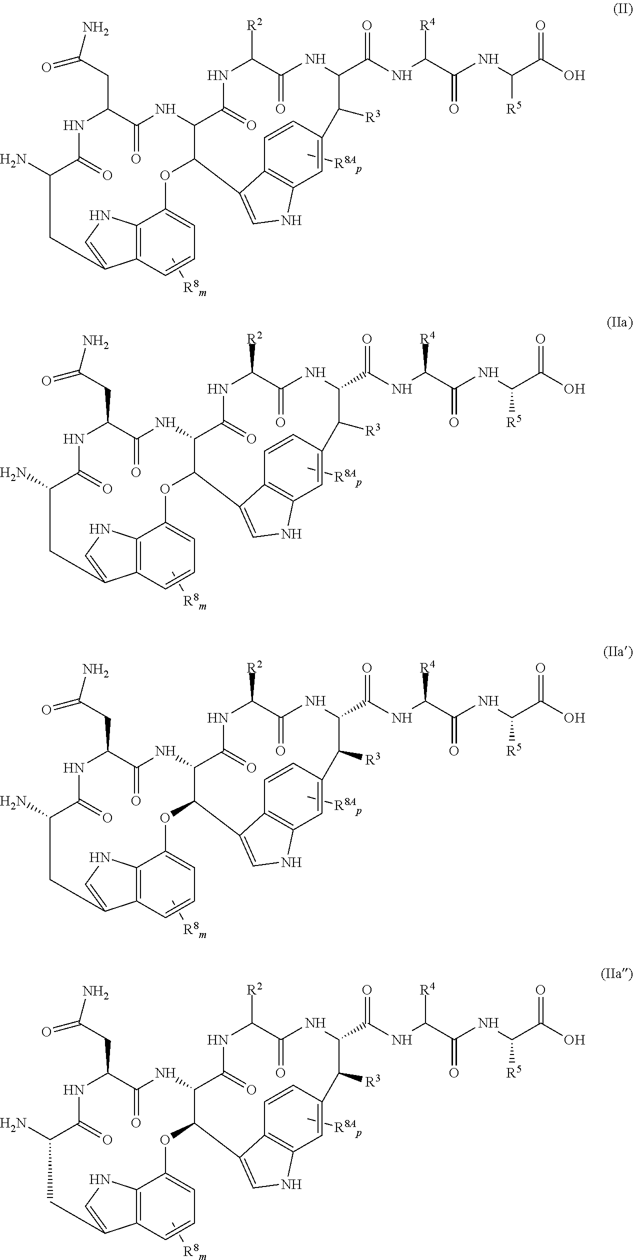

wherein
R² and R⁴ each independently represents H, CH₃, CH₂OH or a group:

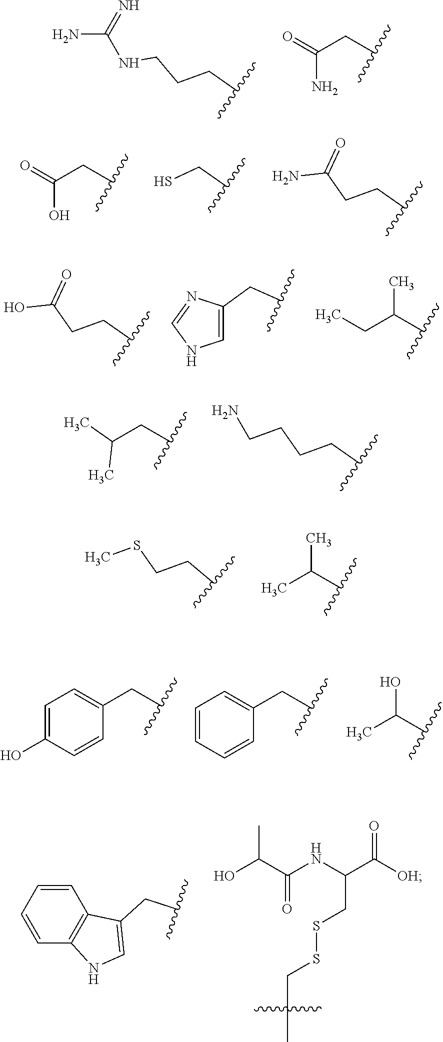

R³ is a group

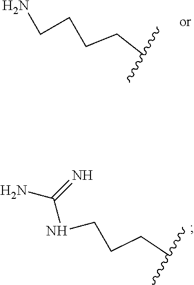 [SMILES: *CCCCN ; *CCCNC(=N)N]

R⁵ is a group of the following formula:

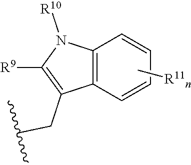

wherein R⁹ is a hydrogen or halogen atom; R¹⁰ is a hydrogen atom or a methyl group; R¹¹ is, at each occasion, independently selected from a halogen atom (especially fluor or chlor) and n is an integer of from 0 to 4 (especially 0 or 1; preferably 0); or a group of formula —CH₃; or a group of the following formula:

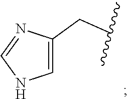 [SMILES: *Cc1c[nH]cn1]

and
wherein the method comprises the steps of:
(a) providing a recombinant host capable of producing said compound of formula (II) (or (IIa), (IIa′) or (IIa″)), wherein said recombinant host harbors at least one synthetic or recombinant nucleic acid encoding a biosynthetic gene cluster (BGC) of the compound, which BGC has:
   (i) a sequence identity to the full-length sequence of SEQ ID NO. 86 of at least 95%, 96%, 97%, 98%, 98.5%, 99%, or 99.5% to 100%; or
   (ii) a sequence completely complementary to any nucleic acid sequence of (i);
(b) cultivating said recombinant host for a time sufficient for said recombinant host to produce the compound of formula (II) (or (IIa), (IIa′) or (IIa″));
(c) isolating the compound from said recombinant host or from cultivation supernatant, thereby producing the compound of formula (II) (or (IIa), (IIa′) or (IIa″)).

In some embodiments of the compound of formula (II), (IIa), (IIa′) or (IIa″), R⁵ is a group of the following formula:

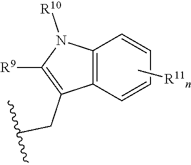

wherein R⁹ is a hydrogen atom; R¹⁰ is a hydrogen atom; R¹¹ is, at each occasion, independently selected from a halogen atom (especially fluor or chlor) and n is an integer of from 0 to 4 (especially 0 or 1; preferably 0).

In some embodiments of the compound of formula (II), (IIa), (IIa′) or (IIa″), R² is a group of the following formula:

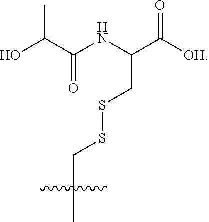 [SMILES: *CSSCC(NC(=O)C(C)O)C(=O)O]

In some embodiments of the compound of formula (II), (IIa), (IIa′) or (IIa″), R⁴ is a group of the following formula:

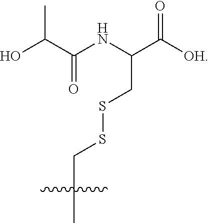 [SMILES: *CSSCC(NC(=O)C(C)O)C(=O)O]

In some embodiments of the compound of formula (II), (IIa), (IIa′) or (IIa″), R³ is a group:

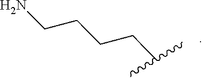 [SMILES: *CCCCN]

In some embodiments of the compound of formula (II), (IIa), (IIa′) or (IIa″), R³ is a group:

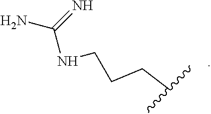 [SMILES: *CCCNC(=N)N]

In some preferred embodiments of the compound of formula (II), (IIa), (IIa′) or (IIa″), R³ is a group:

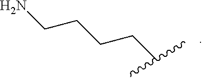 [SMILES: *CCCCN]

In some embodiments, the production method of the compound of formula (II), (IIa), (IIa′) or (IIa″) optionally comprises feeding of one or more halogenated tryptophan(s), preferably halogenated L-tryptophan(s), including, for example, commercially available 5-chloro-L-tryptophan, 7-chloro-L-tryptophan, 6-fluoro-L-tryptophan or 7-fluor-L-tryptophan, to the fermentation broth during cultivation. The one or more halogenated tryptophan(s) can be fed periodically in different concentrations known to the person skilled in the art, and total end concentration of the halogenated tryptophan(s) can, for example, be between 0.5 mM and 10 mM.

In some embodiments, the production method of the compound of formula (II), (IIa), (IIa′) or (IIa″) optionally comprises feeding of one or more halogenated tryptophan(s), preferably halogenated L-tryptophan(s), including, for example, commercially available 5-chloro-L-tryptophan, 7-chloro-L-tryptophan, 6-fluoro-L-tryptophan or 7-fluor-L-tryptophan, to the fermentation broth during cultivation. The one or more halogenated tryptophan(s) can be fed periodically in different concentrations known to the person skilled in the art, and total end concentration of the halogenated tryptophan(s) can, for example, be between 0.5 mM and 10 mM.

In some embodiments, the production method of the compound of formula (II), (IIa), (IIa′) or (IIa″) optionally comprises the cultivation of a host cell which contains vector pUC18-zeo-mx8-corP-trpAB described herein, and the combined feeding of serine (preferably L-serine) and one or more halogenated indol(s) to the fermentation broth during cultivation. In some embodiment, the production method of the compound of formula (II), (IIa), (IIa′) or (IIa″) optionally comprises the cultivation of a recombinant host which contains at least one synthetic or recombinant nucleic acid encoding a biosynthetic gene cluster (BGC) of the compound as described herein, wherein said BCG further includes the trpAB gene of pSTB7 37845 upstream of the darE gene in the BGC, and the feeding of serine (preferably L-serine) and one or more halogenated indol(s) to the fermentation broth during cultivation of the recombinant host. The serine and the one or more halogenated indol(s) can be fed periodically in different concentrations known to the person skilled in the art. For example, serine may be fed in a concentration of 0.5 mM and 15 mM (preferably 8-12 mM, especially 10 mM) and the halogenated indol may be fed in a concentration of 5-10 mM.

As used herein, the term “recombinant host” is intended to refer to a host, the genome of which has been augmented by at least one incorporated DNA sequence. Such DNA sequences include but are not limited to genes that are not naturally present, DNA sequences that are not normally transcribed into RNA or translated into a protein (“expressed”), and other genes or DNA sequences which one desires to introduce into the non-recombinant host. It will be appreciated that typically the genome of a recombinant host described herein is augmented through the stable introduction of one or more recombinant genes. However, autonomous or replicative plasmids or vectors can also be used within the scope of this invention. Moreover, the present invention can be practiced using a low copy number, e.g., a single copy, or high copy number (as exemplified herein) plasmid or vector. “Introduced,” or “augmented” as used herein, are known in the art to mean introduced or augmented by the hand of man.

Generally, the introduced DNA is not originally resident in the host that is the recipient of the DNA, but it is within the scope of the invention to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms such as bacteria (e.g., species of E. coli, Bacillus, Corynebacteria (e.g. C. glutamicum), Lactobacillus (e.g. Lactococcus lactis) or Streptomyces (e.g. S. albus, S. lividans), or a yeast such as Saccharomyces cerevisiae, Saccharomyces pombe, Pichia pastoris or Yarrowia lipolytica), as well as mammalian, insect or plant cell expression systems.

A synthetic or recombinant nucleic acid encoding a biosynthetic gene cluster (BGC) of the peptide compound described herein includes the coding sequence for that polypeptide, operably linked, in sense orientation, to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically includes at least a core (basal) promoter. A regulatory region also may include at least one control element, such as an enhancer sequence, an upstream element or an upstream activation region (UAR). The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region may be present, e.g., introns, enhancers, upstream activation regions, transcription terminators, and inducible elements.

In many cases, the coding sequence for a polypeptide described herein is identified in a species other than the recombinant host, i.e., is a heterologous nucleic acid. The term “heterologous nucleic acid” as used herein, refers to a nucleic acid introduced into a recombinant host, wherein said nucleic acid is not naturally present in said host. Thus, if the recombinant host is a microorganism, the coding sequence can be from other prokaryotic or eukaryotic microorganisms, from plants or from animals. In some case, however, the heterologous nucleic acid may contain a sequence (or parts thereof) that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

It will be appreciated that because of the degeneracy of the genetic code, a number of nucleic acids can encode a particular peptide compound; i.e., for many amino acids, there is more than one nucleotide triplet that serves as the codon for the amino acid. Thus, codons in the coding sequence for a given peptide compound can be modified such that optimal expression in a particular host is obtained, using appropriate codon bias tables for that host (e.g., microorganism). As isolated nucleic acids, these modified sequences can exist as purified molecules and can be incorporated into a vector or a virus for use in constructing modules for recombinant nucleic acid constructs.

Recombinant hosts described herein express darobactin derivatives, e.g. peptide compounds of formula (I), (Ia) or (Ia′) as described above. In one aspect, the present invention relates to a recombinant host harboring a heterologous nucleic acid encoding a darobactin derivative described herein. In particular, the recombinant host described herein harbors a heterologous nucleic acid encoding a peptide compound of formula (I), (Ia) or (Ia′), wherein said heterologous nucleic acid is operably linked to a regulatory region allowing expression in said recombinant host. In an embodiment, the heterologous nucleic acid is an artificial synthetic or recombinant BGC encoding said peptide compounds. Such artificial synthetic or recombinant BGC comprises darA (modified) and darE, which genes have the following predicted functions: modified darA variant (i.e. propeptide of darobactin derivative described herein) and darE: radical S-adenosyl methionine (SAM) methyltransferase (proposed to catalyze the cyclization reactions to connect W₃-K₅ and W₁-W₃). In one embodiment, the artificial synthetic or recombinant BGC further comprises darBCD: having the (predicted) function of an ABC-type trans envelope exporter.

The recombinant host, e.g. microorganism such as bacterial host cell or yeast cell, comprises at least one synthetic or recombinant nucleic acid sequence, which encodes such a synthetic or recombinant BGC of a compound of formula (1), (1a) or (Ia′), and thus being capable of synthesizing a compound according to formula (1), (1a) or (Ia′). The synthetic or recombinant nucleic acid sequence utilized in the production method of the present invention, may, in addition to the nucleic acid sequence encoding the BGC, also include regulatory sequences, such as promoter and translation initiation and termination sequences, and can further include sequences that facilitate stable maintenance in a host cell, i.e., sequences that provide the function of an origin of replication or facilitate integration into host cell chromosomal or other DNA by homologous recombination. The synthetic or recombinant nucleic acid sequence utilized in the production method of the present invention is a synthetic or recombinant nucleic acid sequence, which encodes a BGC of a compound of formula (1) (or of formula (1a), or of formula (1a′)), and has:
(i) a sequence identity to the full-length sequence of SEQ ID NO. 44 of at least 95%, 96%, 97%, 98%, 98.5%, 99%, or 99.5% to 100%, or
(ii) a sequence completely complementary to any nucleic acid sequence of (i).

The term “identity” as used herein refers to a property of sequences that measures their similarity or relationship. Identity is measured by dividing the number of identical residues by the total number of residues and multiplying the product by 100.

The phrases “nucleic acid” or “nucleic acid sequence” as used herein refer to an oligonucleotide, nucleotide, polynucleotide, or to a fragment of any of these, to DNA of genomic or synthetic origin, which may be single-stranded or double-stranded and may represent a sense or antisense strand, natural or synthetic in origin. “Oligonucleotide” includes either a single stranded polydeoxynucleotide or two complementary polydeoxynucleotide strands that may be chemically synthesized. Such synthetic oligonucleotides have no 5′ phosphate and thus will not ligate to another oligonucleotide without adding a phosphate with an ATP in the presence of a kinase. A synthetic oligonucleotide can ligate to a fragment that has not been dephosphorylated. A “coding sequence” of or a “nucleotide sequence encoding” a particular polypeptide or protein, is a nucleic acid sequence which is transcribed and translated into a polypeptide or protein when placed under the control of appropriate regulatory sequences. The nucleic acids used to practice this invention may be isolated from a variety of sources, genetically engineered, amplified, and/or expressed/generated recombinantly. Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, et al, Molecular Cloning: A Laboratory Manual, 2nd Ed., Cold Spring Harbor, N. Y., Vols. 1-3, (1989); Current Protocols in Molecular Biology, Ausubel, ed. John Wiley & Sons, Inc., New York (1997); Laboratory Techniques In Biochemistry And Molecular Biology: Hybridization With Nucleic Acid Probes, Part I. Theory and Nucleic Acid Preparation, Tijssen, ed. Elsevier, N.Y. (1993). A nucleic acid encoding a polypeptide of the invention is assembled in appropriate phase with a leader sequence capable of directing secretion of the translated polypeptide or fragment thereof.

The term “isolated” as used herein means that the material, e.g., a nucleic acid, a polypeptide, a vector, a cell, is removed from its original environment, e.g., the natural environment if it is naturally occurring. For example, a naturally-occurring polynucleotide or polypeptide present in a living animal is not isolated, but the same polynucleotide or polypeptide, separated from some or all of the coexisting materials in the natural system, is isolated. Such polynucleotides can be part of a vector and/or such polynucleotides or peptides can be part of a composition and still be isolated in that such vector or composition is not part of its natural environment.

The term “synthetic” as used herein means that the material, e.g., a nucleic acid, has been synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22: 1859.

The term “recombinant” means that the nucleic acid is adjacent to a “backbone” nucleic acid to which it is not adjacent in its natural environment. Backbone molecules according to the invention include nucleic acids such as cloning and expression vectors, e.g., plasmids, used to maintain or manipulate a nucleic acid insert of interest. Recombinant peptide compounds of the invention, generated from these nucleic acids can be individually isolated or cloned and tested for a desired activity. Any suitable recombinant expression system can be used, including bacterial expression systems.

Also provided is a vector comprising at least one heterologous nucleic acid, e.g., artificial synthetic or recombinant BGC encoding a peptide compound described herein. The vector may be a cloning vector, an expression vector or an artificial chromosome.

As used herein, the term “vector” refers to a nucleic acid molecule capable of transporting another nucleic acid to which it has been linked. Vectors, including cloning and expression vectors, comprise a heterologous nucleic acid described herein or a functional equivalent thereof. Heterologous nucleic acids described herein can be incorporated into a recombinant replicable vector, for example a cloning or expression vector. The vector may be used to replicate the nucleic acid in a compatible host cell. Thus, the invention also provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells are generally known to the person skilled in the art, and examples of suitable host cells are described herein. The vector into which the expression cassette or heterologous nucleic acid described herein is inserted may be any vector which may conveniently be subjected to recombinant DNA procedures, and the choice of the vector will often depend on the host cell into which it is to be introduced. A variety of cloning and expression vectors for use with prokaryotic and eukaryotic hosts are described by Sambrook, et al, Molecular Cloning: A Laboratory Manual, 2nd Ed., Cold Spring Harbor, N. Y., (1989).

A vector according to the invention may be an autonomously replicating vector, i.e. a vector which exists as an extra-chromosomal entity, the replication of which is independent of chromosomal replication, e. g. a plasmid. Also provided herein is an isolated and purified plasmid comprising at least one synthetic or recombinant nucleic acid sequence, which encodes an artificial biosynthetic gene cluster (BGC) of a compound of formula (Ic) (or of formula (Ic′), or of formula (Ic″)), and has:
(i) a sequence identity to the full-length sequence of SEQ ID NO. 1 of at least 95%, 96%, 97%, 98%, 98.5%, 99%, or 99.5% to 100%; or
(ii) a sequence completely complementary to any nucleic acid sequence of (i).

In one embodiment, provided herein is an isolated and purified plasmid comprising at least one synthetic or recombinant nucleic acid sequence, which encodes an artificial BGC encoding a peptide compound of formula (II) (or (IIa), (IIa′) or (IIa″)) described herein above, and has:
(i) a sequence identity to the full-length sequence of SEQ ID NO. 86 of at least 95%, 96%, 97%, 98%, 98.5%, 99%, or 99.5% to 100%; or
(ii) a sequence completely complementary to any nucleic acid sequence of (i).

Alternatively, the vector may be one which, when introduced into a host cell, is integrated into the host cell genome and replicated together with the chromosome(s) into which it has been integrated.

In a preferred embodiment, provided herein is an isolated and purified plasmid comprising at least one synthetic or recombinant nucleic acid sequence, which encodes an artificial BGC encoding a peptide compound of formula (1′) (or (1b), or (1b′)) described herein above, and has:
(i) a sequence identity to the full-length sequence of SEQ ID NO. 44 of at least 95%, 96%, 97%, 98%, 98.5%, 99%, or 99.5% to 100%; or
(ii) a sequence completely complementary to any nucleic acid sequence of (i).

Alternatively, the vector may be one which, when introduced into a host cell, is integrated into the host cell genome and replicated together with the chromosome(s) into which it has been integrated.

The term “plasmid” refers to a circular double stranded DNA loop into which additional DNA segments can be ligated. A plasmid may have a bacterial origin of replication, or may contain an autonomously replicating sequence (ARS) derived from a yeast chromosome, and thus be capable of autonomous replication in a bacterial, or yeast, host cell into which it is introduced. Alternatively, the plasmid may be one which, when introduced into a host cell, is integrated into the host cell genome and replicated together with the genomic DNA into which it has been integrated. The plasmid can be used to replicate the synthetic or recombinant nucleic acid utilized in the invention in a compatible host cell. The plasmid may be recovered from the host cell, and suitable host cells are described below. Moreover, the plasmid may be capable of directing the expression of genes to which it is operatively linked. The plasmid into which the synthetic or recombinant nucleic acid sequence utilized in the invention (expression cassette) is inserted may be any plasmid which may conveniently be subjected to recombinant DNA procedures, and the choice of the plasmid will often depend on the host cell into which it is to be introduced. A variety of cloning and expression plasmids for use with bacterial and yeast hosts are described by Sambrook, et al, Molecular Cloning: A Laboratory Manual, 2nd Ed., Cold Spring Harbor, N. Y., (1989). A plasmid according to the invention may be used in vitro, for example, to transfect or transform a host cell. The plasmid of the invention may comprise one, two or more, for example three, four or five, of the synthetic or recombinant nucleic acid sequences utilized in the production method of the present invention, for example for overexpression. The isolated and purified plasmid of the invention comprises a synthetic or recombinant nucleic acid sequence utilized in the production method of the present invention in a form suitable for expression of the nucleic acid in a bacterial host cell (e.g., bacterium of a species of E. coli, Bacillus, Corynebacteria (e.g. C. glutamicum), Lactobacillus (e.g. Lactococcus lactis) or Streptomyces (e.g. S. albus, S. lividans), or a yeast such as Saccharomyces cerevisiae, Saccharomyces pombe, Pichia pastoris or Yarrowia lipolytica), which means that the plasmid may include one or more regulatory sequences, selected on the basis of the host cells to be used for expression, which is operationally linked to the nucleic acid sequence to be expressed. Particularly useful examples of host cells include bacterial cells of the species E. coli, Bacillus, Corynebacteria, Lactobacillus or Streptomyces, such as E. coli BL21(DE3); and yeast cells of the species S. cerevisiae, Pichia pastoris or Yarrowia lipolytica, such as Pichia pastoris GS115, Pichia pastoris X-33, Pichia pastoris KM71 and Yarrowia lipolytica Po1h (CLIB 882). The plasmids of the invention described herein can be designed for expression of the BGC encoding the peptide compound or the peptide compound of interest in a host cell. Suitable host cells are discussed further in Goeddel, Gene Expression Technology: Methods in Enzymology 185, Academic Press, San Diego, CA (1990). Representative examples of appropriate hosts are generally known in the art, and examples thereof are described herein. Appropriate culture media and conditions for host cells are also known in the art.

Within a plasmid, such as the isolated and purified plasmid of the invention, “operationally linked” is intended to mean that the nucleic acid sequence of interest (i.e. the BGC) is linked to the regulatory sequence(s) in a manner which allows for expression of the nucleic acid sequence (e.g., in an in vitro transcription/translation system or in a host cell when the vector is introduced into the host cell), i.e. the term “operationally linked” refers to a juxtaposition wherein the components described are in a relationship permitting them to function in their intended manner. A regulatory sequence such as a promoter, enhancer or other expression regulation signal “operationally linked” to a coding sequence is positioned in such a way that expression of the coding sequence is achieved under condition compatible with the control sequences or the sequences are arranged so that they function in concert for their intended purpose, for example transcription initiates at a promoter and proceeds through the DNA sequence encoding the polypeptide.

The term “regulatory sequence” or “control sequence” is intended to include promoters, operators, enhancers, attenuators and other expression control elements (e.g., polyadenylation signal). Such regulatory sequences are described, for example, in Goeddel; Gene Expression Technology: Methods in Enzymology 185, Academic Press, San Diego, CA (1990). The term regulatory or control sequence includes those sequences, which direct constitutive expression of a nucleotide sequence in many types of host cells and those, which direct expression of the nucleotide sequence only in a certain host cell (e.g. genus-specific regulatory sequences).

A plasmid or expression construct for a given host cell may thus comprise the following elements operationally linked to each other in a consecutive order from the 5′-end to 3′-end relative to the coding strand of the sequence encoding a polypeptide for production by the method of the invention: (i) a promoter sequence capable of directing transcription of the nucleotide sequence encoding the polypeptide in the given host cell; (ii) optionally, a signal sequence capable of directing secretion of the polypeptide from the given host cell into a culture medium; (iii) optionally, a sequence encoding for a C-terminal, N-terminal or internal epitope tag sequence or a combination of the aforementioned allowing purification, detection or labeling of the polypeptide; (iv) a nucleic acid sequence of the invention encoding a polypeptide of the invention; and preferably also (v) a transcription termination region (terminator) capable of terminating transcription downstream of the nucleotide sequence encoding the polypeptide.

Particular named bacterial promoters include lad, ntpII, lacZ, pTuf, Eftu, T3, T7, SP6, K1F, tac, tet, gpt, p_{BAD}, lambda P_{R}, P_{L} and trp. Examples of Saccharomyces cerevisiae promoters include P_{ADH1}, P_{GAL1}, P_{CYC1}, P_{GPD} (also called P_{TDH3}), P_{ZEV} P_{ACT1} P_{HXK1}, P_{YGR243}, P_{HXT4}, P_{HXT7} P_{TEF1}, P_{TPI1}, P_{PGK1}, P_{TDH3} and P_{PYK1}. In a preferred embodiment, the S. cerevisiae promoter is P_{ADH1}. Examples of Pichia pastoris promoters include P_{AOX1}, P_{GAP}, P_{ADH3}, P_{PGK1}, P_{DAS}, P_{FLD1}, and P_{PEX8}; and preferred embodiments include P_{AOX1}, P_{GAP}, P_{ADH3}, and P_{PGK1}. Examples of Yarrowia lipolytica promoters include PmLEU2, pPOX2, pEYK1, pLIP2, pXPR2 and pLEU2; and preferred embodiments include PmLEU2. Examples of Streptomyces albus and Streptomyces lividans promoters include KasOp, psf ermEp, SF14p, PtipA, PkasO*_{R15}, and Ptac. In a preferred embodiment the S. albus and S. lividans promoters are KasOp, psf and ermEp.

Selection of the appropriate plasmid and promoter is well within the level of ordinary skill in the art. Downstream of the synthetic or recombinant nucleic acid sequence utilized in the production method of the present invention there may be a 3′ untranslated region containing one or more transcription termination sites (e. g. a terminator). The origin of the terminator is less critical. The terminator can, for example, be native to the DNA sequence encoding the peptide compound. Preferably, the terminator is endogenous to the host cell (in which the nucleotide sequence encoding the polypeptide is to be expressed). In the transcribed region, a ribosome binding site for translation may be present. The coding portion of the mature transcripts expressed by the constructs will include a translation initiating AUG (or TUG or GUG in prokaryotes) at the beginning and a termination codon appropriately positioned at the end of the peptide compound to be translated or processed.

Enhanced expression of a polynucleotide of the invention or a heterologous nucleic acid described herein may also be achieved by the selection of heterologous regulatory regions, e.g. promoter, secretion leader and/or terminator regions, which may serve to increase expression and, if desired, secretion levels of the protein of interest from the expression host and/or to provide for the inducible control of the expression of the peptide compound. It will be appreciated by those skilled in the art that the design of the plasmid can depend on such factors as the choice of the host cell to be transformed, the level of expression of protein desired, etc. The plasmids of the invention can be introduced into the recombinant host, e.g., microorganism (e.g., bacterial host cells) to thereby produce peptides, encoded by nucleic acids as described herein. Preferably, plasmids of the invention are capable of autonomous replication in a microorganism, such as a bacterium (e.g., bacterium of the species of E. coli, Bacillus, Corynebacteria (e.g. C. glutamicum), Lactobacillus (e.g. Lactococcus lactis) or Streptomyces (e.g. S. albus, S. lividans), or a yeast such as Saccharomyces cerevisiae, Saccharomyces pombe, Pichia pastoris or Yarrowia lipolytica). Preferably, plasmids of the invention are capable of autonomous replication in bacteria of the species Lactobacillus or E. coli, preferably E. coli.

The plasmids of the invention can be designed for expression of the BGC or the peptide compound of interest in bacterial cells such as E. coli or Bacillus strains, e.g., Lactobacillus. Suitable host cells are discussed further in Goeddel, Gene Expression Technology: Methods in Enzymology 185, Academic Press, San Diego, CA (1990). Representative examples of appropriate hosts are described hereafter. Appropriate culture media and conditions for the above-described host cells are known in the art.

As set out above, the term “control sequences” or “regulatory sequences” is defined herein to include at least any component, which may be necessary and/or advantageous for the expression of a peptide compound described herein. Any control sequence may be native or foreign to the nucleic acid sequences encoding a BGC for production of a peptide compound described herein. Such control sequences may include, but are not limited to, a promoter, a leader, optimal translation initiation sequences (as described in Kozak, 1991, J. Biol. Chem. 266:19867-19870), a secretion signal sequence, a pro-peptide sequence, a polyadenylation sequence, a transcription terminator. At a minimum, the control sequences typically include a promoter, and transcriptional and translational stop signals. A stably transformed microorganism is one that has had one or more DNA fragments introduced such that the introduced molecules are maintained, replicated and segregated in a growing culture. Stable transformation may be due to multiple or single chromosomal integration(s) or by (an) extrachromosomal element(s) such as (a) plasmid vector(s). A plasmid vector is capable of directing the expression of peptide compounds encoded by particular DNA fragments. Expression may be constitutive or regulated by inducible (or repressible) promoters that enable high levels of transcription of functionally associated DNA fragments encoding specific peptide compounds.

Plasmids of the invention may also include a selectable marker gene to allow for the selection of bacterial strains that have been transformed, e.g., genes, which render the bacteria resistant to drugs such as chloramphenicol, erythromycin, kanamycin, neomycin, apramycin, tetracycline, zeocin, hygromycin, as well as ampicillin and other penicillin derivatives like carbenicillin. Selectable markers can also include biosynthetic genes, such as those in the histidine, tryptophan and leucine biosynthetic pathways.

The appropriate polynucleotide sequence may be inserted into the plasmid by a variety of procedures. In general, the polynucleotide sequence is ligated to the desired position in the plasmid following digestion of the insert and the vector with appropriate restriction endonucleases. Alternatively, blunt ends in both the insert and the vector may be ligated. A variety of cloning techniques are disclosed in Ausubel et al. Current Protocols in Molecular Biology, John Wiley 503 Sons, Inc. 1997 and Sambrook et al, Molecular Cloning: A Laboratory Manual 2nd Ed., Cold Spring Harbor Laboratory Press (1989). The polynucleotide sequence may also be cloned using homologous recombination techniques including in vitro as well as in vivo recombination. Such procedures and others are deemed to be within the scope of those skilled in the art.

Further provided is a recombinant host comprising a heterologous nucleic acid encoding a darobactin derivative of the invention, i.e. a compound of formula (I), (Ia) or (Ia′) as described above. In an embodiment, the recombinant host is a microorganism, e.g., a bacterium such as a bacterium of the species of E. coli, Bacillus, Corynebacteria (e.g. C. glutamicum), Lactobacillus (e.g. Lactococcus lactis) or Streptomyces (e.g. S. albus, S. lividans), or a yeast such as Saccharomyces cerevisiae, Saccharomyces pombe, Pichia pastoris or Yarrowia lipolytica. The heterologous nucleic acid may be part of a vector, such as a plasmid, contained in the microorganism. The vector, e.g. an expression vector, such as a plasmid, can be introduced into the microorganism, e.g. a bacterial host cell or yeast host cell of a species described herein, by any of a variety of techniques known to those skilled in the art, including transformation or transfection. The invention also provides bacterial host cells, i.e. transformed cells comprising at least one synthetic or recombinant nucleic acid sequence, which encodes a biosynthetic gene cluster for production of a compound of formula (Ic) (or formula (Id) or (Ie)), or a plasmid of the invention. The host cell may be any bacterial host cell familiar to those skilled in the art, e.g. an Escherichia coli or Lactobacillus cell, preferably an E. coli cell, and more preferably an E. coli BL21(DE3) cell. The plasmid can be introduced into the bacterial host cell using any of a variety of techniques, including transformation or transfection. Particular methods include calcium phosphate transfection, DEAE-Dextran mediated transfection, lipofection, or electroporation (Davis, L., Dibner, M., Battey, I., Basic Methods in Molecular Biology, (1986)).

Where appropriate, the engineered host cells can be cultured in conventional nutrient media modified as appropriate for activating promoters, selecting transformants or amplifying the synthetic or recombinant nucleic acid utilized in the production method of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired peptide compound or derivatives thereof. The culture conditions such as temperature, pH and the like, are apparent to the ordinarily skilled artisan, and examples are described herein. The host cell, when cultured under suitable conditions, is capable of producing darobactin A or darobactin derivatives, i.e. peptide compounds according to formula (Ic) (or (Ic′), or (Ic″)) or any of the other compounds described herein (especially darobactin derivatives of the present invention), that it otherwise does not produce, or produces at a significantly lower level, in the absence of a modification according to the invention. Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed peptide compound or derivatives thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. If desired, high performance liquid chromatography (HPLC) can be employed for final purification steps.

The terms “protein”, “polypeptide”, “peptide” as used herein define an organic compound made of two or more amino acid residues arranged in a linear chain or cyclic structure, wherein individual amino acids in the organic compound are linked by peptide bonds, i.e. an amide bond formed between adjacent amino acid residues. By convention, the primary structure of a protein is reported starting from the amino-terminal (N) end to the carboxyl-terminal (C) end.

### BRIEF DESCRIPTION OF THE FIGURES

FIG. 1 graphically depicts the general structure of a darobactin expression vector, i.e. plasmid, of the invention. Table 1 below summarizes the restriction sites (R-sites) that are present in the expression vector for modification by cloning techniques.

TABLE 1
Restriction sites (R-sites) with appropriate enzyme and function

| R-site | Enzyme | Function |
|---|---|---|
| R₂ | MauBI | pNOSO cloning |
| R₃ | AgeI | Exchange/introduction of element(s) |
| R₄ | MreI | T7 promoter exchange |
| R₅ | XmaJI | T7 promoter exchange |
| R₆ | NdeI | T7 promoter & darA exchange |
| R₇ | PstI | darA exchange |
| R₈ | BamHI | darA exchange |
| R₉ | NotI | ntpII promoter exchange |
| R₁₀ | XhoI | ntpII promoter exchange |
| R₁₁ | SpeI | introduction of alternative and/or additional genes |
| R₁₂ | PacI | introduction of alternative and/or additional genes |
| R₁₃ | SgrDI | pNOSO cloning |

FIG. 2 graphically depicts a biosynthetic gene cluster (BGC) responsible for the production of darobactin A, and further shows the DNA sequence encoding the darobactin A propeptide core sequence, its corresponding linear propeptide amino acid sequence and the applied numbering of the amino acids.

### EXAMPLES

Generation of Expression Vector pNOSOdarA-E-DA

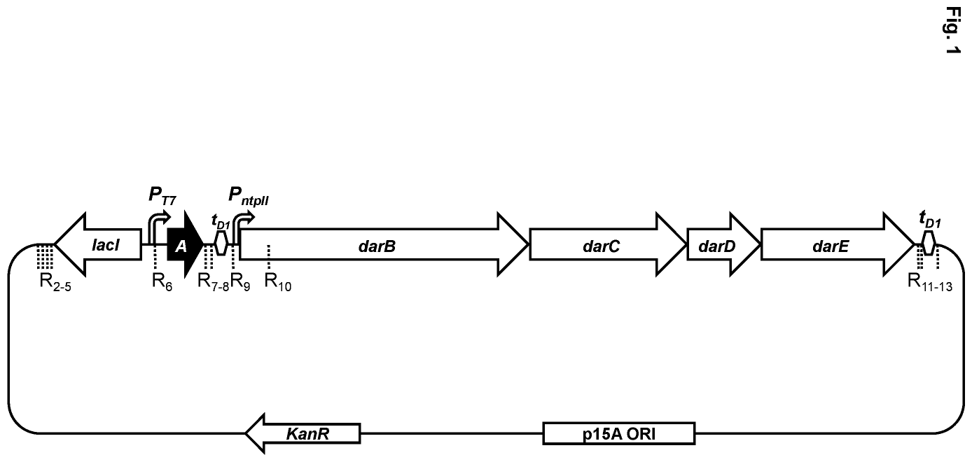

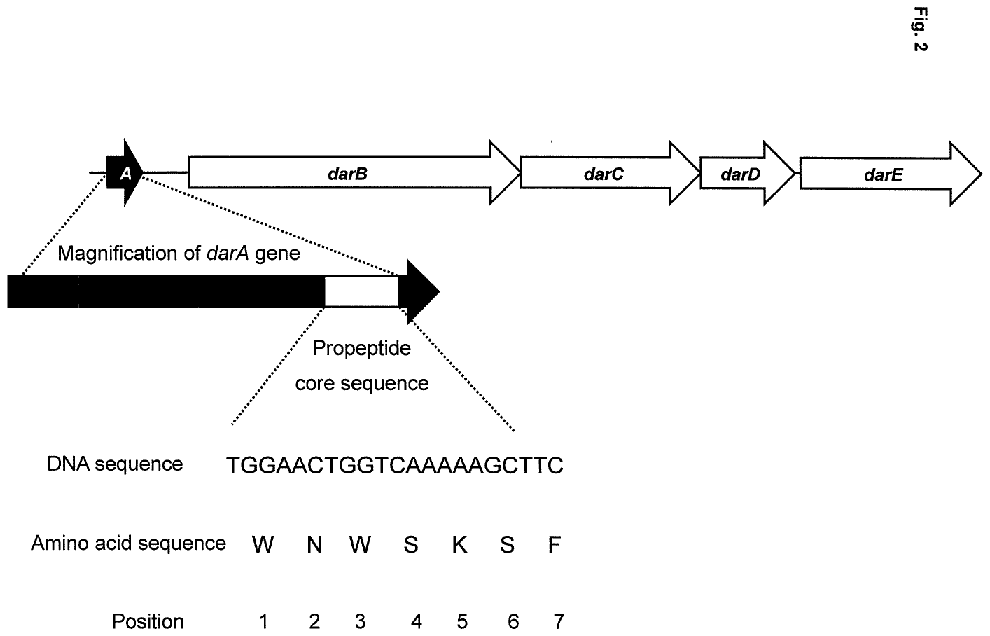

Plasmid expression vector pNOSOdarA-E-DA (deposited at DSMZ on 10 Feb. 2021; DSM 33801) was generated from pNOSO vector and an artificial synthetic sequence comprising a BGC for the production of darobactin A. Both, pNOSO and the synthetic sequence comprising the BGC, were designed in silico, then chemically synthesized and assembled using restriction/ligation-based cloning techniques. The pNOSO vector harbours p15A ori, kanamycin resistance cassette (KanR), CEN6/ARS4 (yeast ori) and URA3 (yeast counter selection marker). The artificial synthetic sequence comprises lactose-dependent repressor gene (lacI), T7 promoter (P_{T7}), constitutive promoter (P_{ntpII}), termination cassettes (t_{D1}) and a BGC of darobactin A. Additionally, point mutations were introduced into pNOSO and the synthetic BGC to generate unique R-sites for cloning purposes. After chemical synthesis of the vector pNOSO and the artificial synthetic sequence comprising the darobactin BGC, the pNOSOdarA-E-DA expression construct was generated by restriction/ligation-based cloning techniques. The CEN6/ARS4 sequence, the URA3 sequence and the relE gene were removed during generation of plasmid expression vector pNOSOdarA-E-DA. The detailed vector organization is graphically depicted in FIG. 1; for details of the R-sites see Table 1 above.

Generation of Artificial darA Gene Fragments and Plasmid Expression Vectors

For the generation of new artificial darobactin derivatives with altered amino acid sequences compared to darobactin A, the propeptide sequences in the darA gene were modified in silico by exchange of nucleotides and/or codons. Artificial darA gene fragments containing the modified propetide sequences were chemically synthesized and cloned into pNOSOdarA-E-DA by replacing darA with a modified darA gene fragment using restriction/ligation-based cloning techniques. Using this strategy, plasmid expression vectors with altered propeptide sequences in the darA gene were generated (e.g. pNOSOdarA-E-DB to pNOSOdarA-E-DE and pNOSOdarA-E-D1 to pNOSOdarA-E-D17). Table 2 shows modified propeptide DNA sequences of artificial darA gene fragments and their corresponding propetide amino acid sequence, which can be used for the production of darobactin derivatives.

TABLE 2
Propeptide DNA and protein sequences
of darobactin derivatives.

| Darobactin | DNA Propeptide | Propeptide amino |
|---|---|---|
| derivative | core sequence | acid sequence |
| B | TGGAACTGGACCAAACGATTC | WNWTKRF |
| C | TGGTCATGGTCAAGAAGCTTC | WSWSRSF |
| D | TGGAACTGGTCAAGAAGCTTC | WNWSRSF |
| E | TGGTCATGGTCAAAAAGCTTC | WSWSKSF |
| D1 | TGGTCATGGACCAAACGATTC | WSWTKRF |
| D2 | TGGTCATGGACCAGAAGCTTC | WSWTKRF |
| D4 | TGGAACTGGACCAAAAGCTTC | WNWTKSF |
| D5 | TGGTGCTGGTCAAAAAGCTTC | WCWSKSF |
| D6 | TGGAACTGGTGCAAAAGCTTC | WNWCKSF |
| D7 | TGGTGCTGGTGCAAAAGCTTC | WCWCKSF |
| D8 | TGGAACTGGTCAAAATGCTTC | WNWSKCF |
| D9 | TGGAACTGGTCAAAAAGCTGG | WNWSKSW |
| D10 | TGGAACTGGTCAAAAAGCTAC | WNWSKSY |
| D11 | TGGCAGTGGTCAAAAAGCTTC | WQWSKSF |
| D12 | TGGACCTGGTCAAAAAGCTTC | WTWSKSF |
| D13 | TGGGCATGGTCAAAAAGCTTC | WAWSKSF |
| D14 | TGGAACTGGGCAAAAAGCTTC | WNWAKSF |
| D15 | TGGAACTGGTCAGCAAGCTTC | WNWSASF |
| D16 | TGGAACTGGTCAAAAGCTTTC | WNWSKAF |
| D17 | TGGAACTGGTCAAAAAGCGCA | WNWSKSA |

The correctness of the generated plasmids was verified by restriction hydrolysis followed by agarose gel electrophoresis and DNA sequencing. Designation of the plasmid expression vectors corresponds to nomenclature of the darobactin derivatives, e.g., pNOSOdarA-E-D9 harbours the propeptide sequence of darobactin D9 (i.e. WNWSKSW) and pNOSOdarA-E-D17 harbours the propeptide sequence of darobactin derivative 17 (i.e. WNWSKSA); both of which have been deposited at DSMZ on 10 Feb. 2021; DSM 33802 (pNOSOdarA-E-D9) and DSM 33803 (pNOSOdarA-E-D17)).

### Heterologous Expression and Production

For heterologous production of darobactin A (DA) and darobactin derivatives, a plasmid expression vector as described above was transformed into E. coli BL21 (DE3) cells by electroporation, and the respective transformed host cells were grown under controlled conditions as, for example, described below.

### Fermentation/Cultivation of Host Cells

### Day 1

Inoculation of a single clone of E. coli BL21 (DE3) cells harbouring plasmid pNOSOdarA-E-DA or pNOSOdarA-E with modified darA gene in 10 ml LB medium including 30 μg/ml Kanamycin (Kan30)

### Day 2

Inoculation of 50 ml FM medium (1.254% K2HPO4, 0.231% KH2PO4, 0.4% D(+)-glucose, 0.1% NH₄Cl, 1.2% yeast extract, 0.5% NaCl and 0.0492% MgSO4(*7H2O); pH 7.1) including Kan30 and 1 mg/l VitaminB12 with 1% (v/v) of preculture
Incubation for 3 days at 30° C., 180 rpm (Infors HT)

### Separation and Retaining of Compounds

### Day 5

Harvest supernatant by centrifugation at 8,000 g for 10 min at 4° C.
Incubation of supernatant with 0.05% XAD16N resin (Sigma Aldrich) (w/v) under agitation for 2 h
Harvest of XAD16N resin and elution of bound compounds from XAD16N resin with 10 ml 80% Methanol under shaking for 1.5 h
Drying of eluate in a rotary evaporator
Solubilization of evaporated extract in 0.5 ml 50/50 methanol/water mixture
HPLC-MS analysis to verify the production of peptide compound of interest, e.g., darobactin A or darobactin derivative; characteristic ions for darobactins in tandem MS mode are m/z 160.075 and a prominent [M-H₂O+2H]²⁺ ion.

In case of production of compounds for antimicrobial testing with the minimum inhibitory concentration assays described below, no antibiotic was added to the expression cultures as a selection marker.

HPLC purification was carried out on a Waters Autopurifier (Eschborn, Germany) high pressure gradient system, equipped with 2545 binary gradient module, SFO system fluidics organizer, 2767 sample manager and a 2998 photodiode array detector coupled to a 3100 single quadrupole mass spectrometer operated in positive ion mode. Source and voltage settings for the MS were as follows: mass range, m/z 300-1000; scan duration, 1 s; points per Dalton, 4; capillary voltage, 3.5 kV; cone voltage, 30 V; extractor voltage 3 V; RF lens, 0.1 V; source temperature 120° C., desolvation temperature, 250° C.; desolvation gas flow, 400 L/h; cone gas flow, 50 L/h; ion counting threshold, 30. Separation was carried out on a Waters X-Bridge prep C-18 5 μm ODB, 150×19 mm column using ACN+0.1% FA as B and H₂O+0.1% FA as A and a flow rate of 25 ml/min. Compound detection was carried out by detection of the [M+2H]²⁺. Second purification step was carried out on the same instrument on an Phenomenex Kinetex 5u Biphenyl 100A 250×21.2 mm using ACN+0.1% FA as B and H₂O+0.1% FA as A and a flow rate of 25 ml/min. Where necessary, a Dionex Ultimate 3000 SDLC low pressure gradient system on a Waters CSH Phenyl-Hexyl 250 mm×10 mm 5 μm dp column with the eluents H₂O+0.1% FA as A and ACN+0.1% FA as B, a flow rate of 5 ml/min and a column thermostated at 30° C. was used for further purification of peptide compounds of interest. Peptide compounds of interest, e.g. darobactin A and darobactin derivatives, can be detected by UV absorption at 254 nm.

Following the above protocol, darobactin A was, for example, heterologously produced by expression of a modified artificial plasmid expression vector comprising propeptide sequence A (see, e.g., FIG. 2) in E. coli BL21(DE3) cells, and obtained as a slightly yellow solid.

### Example Compounds

Exemplified example compounds obtained by heterologous expression of the modified BGCs according to the present invention are shown in Table 3 below.

TABLE 3
Produced example compounds

| Darobactin/ | | |
|---|---|---|
| Darobactin | | Mass (m/z)* |
| derivative | Chemical structure based on exact mass and MS2 fragmentation pattern | [M + 2H]² |
| A | 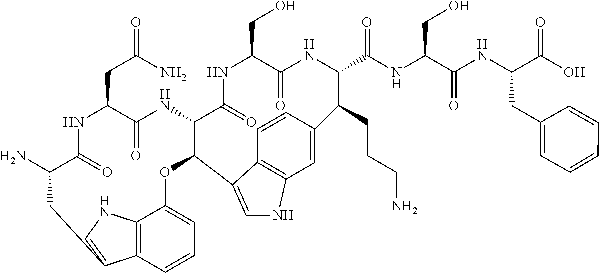 [SMILES: NCCC[C@H]1c2ccc3c(c[nH]c3c2)[C@H]2Oc3cccc4c(c[nH]c34)C[C@H](N)C(=O)N[C@@H](CC(N)=O)C(=O)N[C@@H]2C(=O)N[C@@H](CO)C(=O)N[C@@H]1C(=O)N[C@@H](CO)C(=O)N[C@@H](Cc1ccccc1)C(=O)O] | 483.70 |
| B | 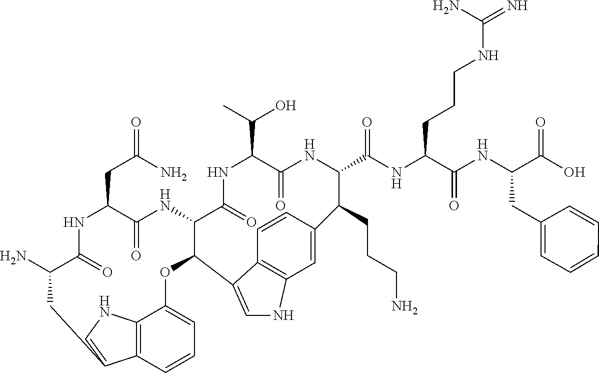 [SMILES: CC(O)[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CCCNC(=N)N)C(=O)N[C@@H](Cc2ccccc2)C(=O)O)NC1=O] | 525.25 |
| B* | 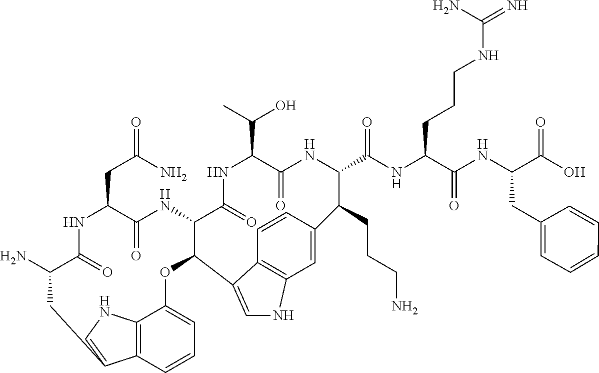 [SMILES: CC(O)[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CCCNC(=N)N)C(=O)N[C@@H](Cc2ccccc2)C(=O)O)NC1=O] | 525.25 |
| D | 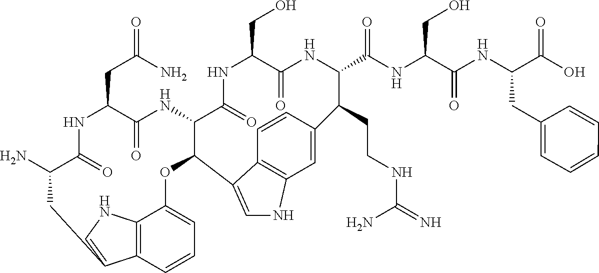 [SMILES: N=C(N)NCC[C@H]1c2ccc3c(c[nH]c3c2)[C@H]2Oc3cccc4c(c[nH]c34)C[C@H](N)C(=O)N[C@@H](CC(N)=O)C(=O)N[C@@H]2C(=O)N[C@@H](CO)C(=O)N[C@@H]1C(=O)N[C@@H](CO)C(=O)N[C@@H](Cc1ccccc1)C(=O)O] | 497.71 |
| E | 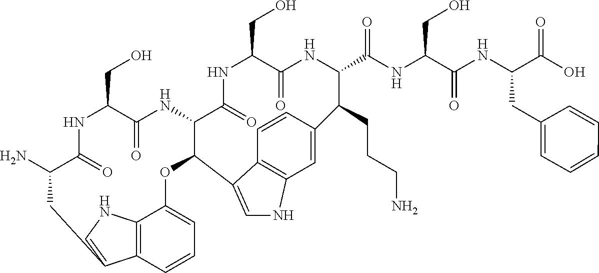 [SMILES: NCCC[C@H]1c2ccc3c(c[nH]c3c2)[C@H]2Oc3cccc4c(c[nH]c34)C[C@H](N)C(=O)N[C@@H](CO)C(=O)N[C@@H]2C(=O)N[C@@H](CO)C(=O)N[C@@H]1C(=O)N[C@@H](CO)C(=O)N[C@@H](Cc1ccccc1)C(=O)O] | 470.20 |
| D1 | 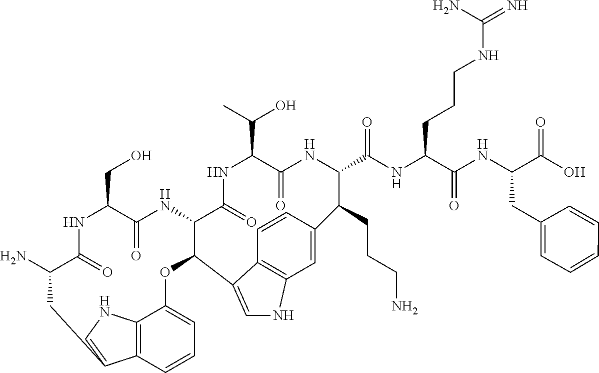 [SMILES: CC(O)[C@@H]1NC(=O)[C@H]2NC(=O)[C@H](CO)NC(=O)[C@@H](N)Cc3c[nH]c4c(cccc34)O[C@@H]2c2c[nH]c3cc(ccc23)[C@H](CCCN)[C@@H](C(=O)N[C@@H](CCCNC(=N)N)C(=O)N[C@@H](Cc2ccccc2)C(=O)O)NC1=O] | 511.75 |
| D1* | 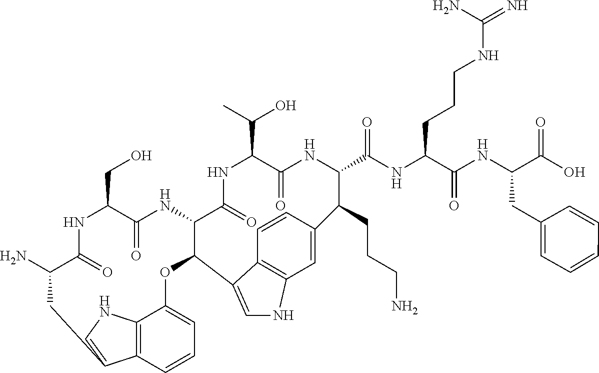 [SMILES: CC(O)[C@@H]1NC(=O)[C@H]2NC(=O)[C@H](CO)NC(=O)[C@@H](N)Cc3c[nH]c4c(cccc34)O[C@@H]2c2c[nH]c3cc(ccc23)[C@H](CCCN)[C@@H](C(=O)N[C@@H](CCCNC(=N)N)C(=O)N[C@@H](Cc2ccccc2)C(=O)O)NC1=O] | 511.75 |
| D2 | 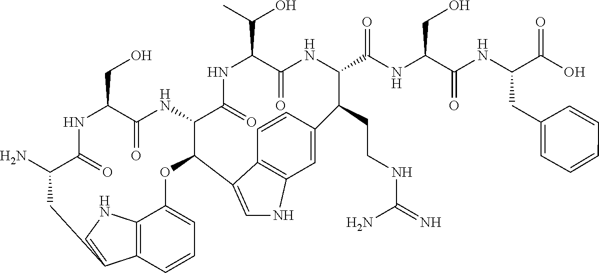 [SMILES: CC(O)[C@@H]1NC(=O)[C@H]2NC(=O)[C@H](CO)NC(=O)[C@@H](N)Cc3c[nH]c4c(cccc34)O[C@@H]2c2c[nH]c3cc(ccc23)[C@H](CCNC(=N)N)[C@@H](C(=O)N[C@@H](CO)C(=O)N[C@@H](Cc2ccccc2)C(=O)O)NC1=O] | 491.21 |
| D2* | 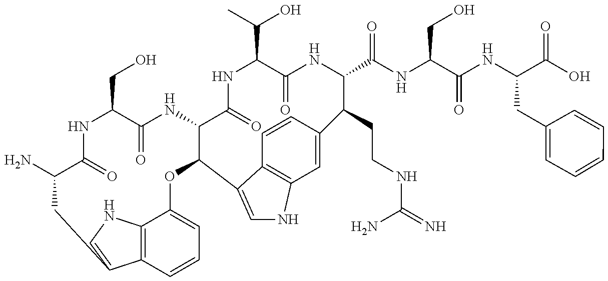 [SMILES: CC(O)[C@@H]1NC(=O)[C@H]2NC(=O)[C@H](CO)NC(=O)[C@@H](N)Cc3c[nH]c4c(cccc34)O[C@@H]2c2c[nH]c3cc(ccc23)[C@H](CCNC(=N)N)[C@@H](C(=O)N[C@@H](CO)C(=O)N[C@@H](Cc2ccccc2)C(=O)O)NC1=O] | 491.21 |
| D4 | 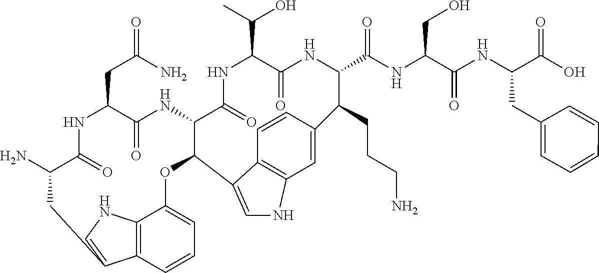 [SMILES: CC(O)[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CO)C(=O)N[C@@H](Cc2ccccc2)C(=O)O)NC1=O] | 490.71 |
| D4* | 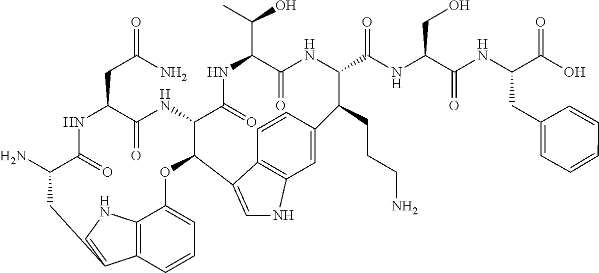 [SMILES: C[C@@H](O)[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CO)C(=O)N[C@@H](Cc2ccccc2)C(=O)O)NC1=O] | 490.71 |
| D9 | 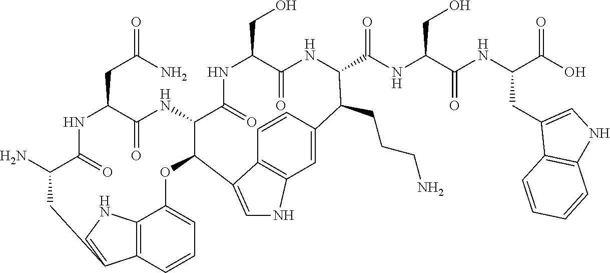 [SMILES: NCCC[C@H]1c2ccc3c(c[nH]c3c2)[C@H]2Oc3cccc4c(c[nH]c34)C[C@H](N)C(=O)N[C@@H](CC(N)=O)C(=O)N[C@@H]2C(=O)N[C@@H](CO)C(=O)N[C@@H]1C(=O)N[C@@H](CO)C(=O)N[C@@H](Cc1c[nH]c2ccccc12)C(=O)O] | 503.21 |
| D10 | 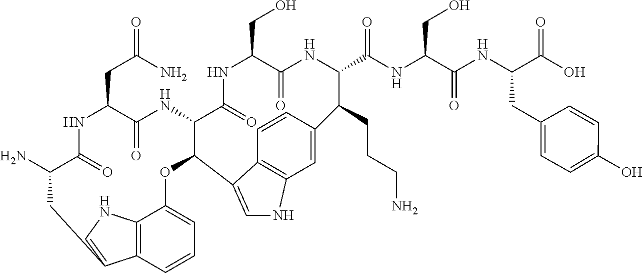 [SMILES: NCCC[C@H]1c2ccc3c(c[nH]c3c2)[C@H]2Oc3cccc4c(c[nH]c34)C[C@H](N)C(=O)N[C@@H](CC(N)=O)C(=O)N[C@@H]2C(=O)N[C@@H](CO)C(=O)N[C@@H]1C(=O)N[C@@H](CO)C(=O)N[C@@H](Cc1ccc(O)cc1)C(=O)O] | 491.70 |
| D11 | 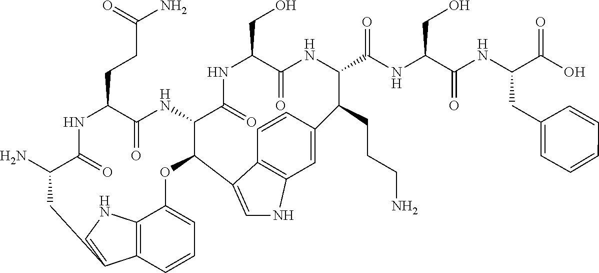 [SMILES: NCCC[C@H]1c2ccc3c(c[nH]c3c2)[C@H]2Oc3cccc4c(c[nH]c34)C[C@H](N)C(=O)N[C@@H](CCC(N)=O)C(=O)N[C@@H]2C(=O)N[C@@H](CO)C(=O)N[C@@H]1C(=O)N[C@@H](CO)C(=O)N[C@@H](Cc1ccccc1)C(=O)O] | 490.71 |
| D12 | 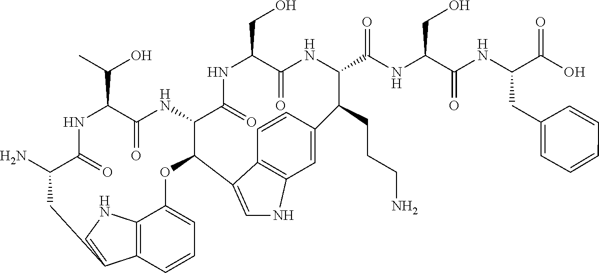 [SMILES: CC(O)[C@@H]1NC(=O)[C@@H](N)Cc2c[nH]c3c(cccc23)O[C@@H]2c3c[nH]c4cc(ccc34)[C@H](CCCN)[C@@H](C(=O)N[C@@H](CO)C(=O)N[C@@H](Cc3ccccc3)C(=O)O)NC(=O)[C@H](CO)NC(=O)[C@H]2NC1=O] | 477.21 |
| D12* | 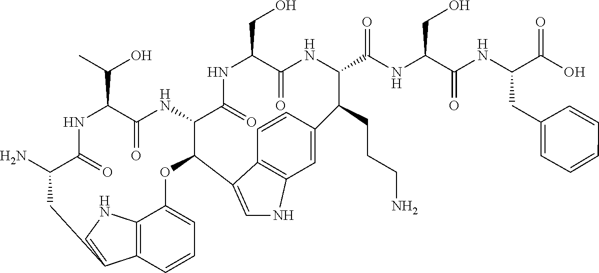 [SMILES: CC(O)[C@@H]1NC(=O)[C@@H](N)Cc2c[nH]c3c(cccc23)O[C@@H]2c3c[nH]c4cc(ccc34)[C@H](CCCN)[C@@H](C(=O)N[C@@H](CO)C(=O)N[C@@H](Cc3ccccc3)C(=O)O)NC(=O)[C@H](CO)NC(=O)[C@H]2NC1=O] | 477.21 |
| D14 | 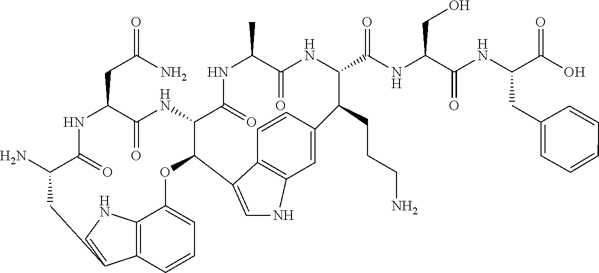 [SMILES: C[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CO)C(=O)N[C@@H](Cc2ccccc2)C(=O)O)NC1=O] | 475.71 |
| D15 | 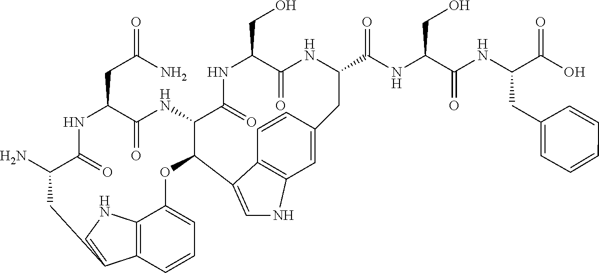 [SMILES: NC(=O)C[C@@H]1NC(=O)[C@@H](N)Cc2c[nH]c3c(cccc23)O[C@@H]2c3c[nH]c4cc(ccc34)C[C@@H](C(=O)N[C@@H](CO)C(=O)N[C@@H](Cc3ccccc3)C(=O)O)NC(=O)[C@H](CO)NC(=O)[C@H]2NC1=O] | 455.17 |
| D16 | 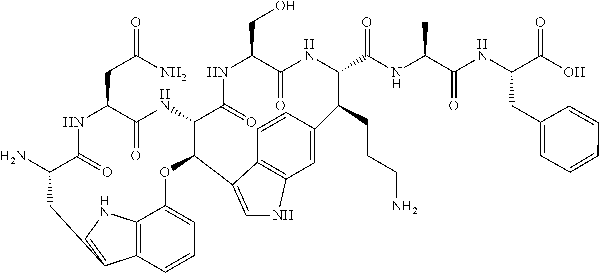 [SMILES: C[C@H](NC(=O)[C@H]1NC(=O)[C@H](CO)NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@@H]1CCCN)C(=O)N[C@@H](Cc1ccccc1)C(=O)O] | 475.71 |
| D17 | 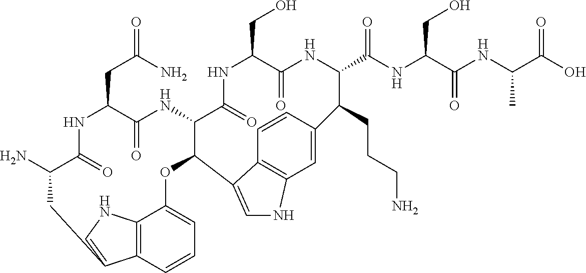 [SMILES: C[C@H](NC(=O)[C@H](CO)NC(=O)[C@H]1NC(=O)[C@H](CO)NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@@H]1CCCN)C(=O)O] | 445.69 |

*All observed [M + 2H]²⁺ masses correlate with the expected [M + 2H]²⁺ masses.

### Assessment of Antimicrobial Activity of Darobactin A and Darobactin Derivative 9

All microorganisms were handled according to standard procedures as recommended by the depositor. Strains were obtained from the German Collection of Cell Cultures and Microorganisms (Klebsiella pneumoniae DSM-30104, Acinetobacter baumannii DSM-30008), the American Type Culture Collection (Staphylococcus aureus ATCC-29213, Escherichia coli ATCC-25922, Enterococcus faecalis ATCC-29212), or were part of our internal strain collection (Pseudomonas aeruginosa PAO1).

All samples were tested according to standard procedures to establish minimum inhibitory concentration (MIC) by the microbroth dilution method. Bacteria were grown on solid media (CASO agar) and single colonies were collected with sterile cotton swabs and resuspended in saline solution (0.9% NaCl) to obtain McFarland 0.5. The bacterial suspension was diluted 1:100 in cation-adjusted Mueller-Hinton broth to obtain a final inoculum of approximately 10⁶ CFU/mL. Serial dilutions of the test samples were prepared in cation-adjusted Mueller-Hinton broth in sterile 96-well microtiter plates in a total volume of 75 μL. The prepared cell suspension was added (75 μL) and the plates were incubated at either 30° C. or 37° C. for at least 24 h under static conditions. Plates were then checked visually and MIC was determined as lowest concentration where no visible growth was observed. An extract of sterile fermentation medium (FM medium) and E. coli BL21 (DE3) cells harbouring plasmid expression vector pNOSO without BGC were used as negative controls. Results of darobactin A and darobactin derivative 9 produced in accordance with the invention compared to data published in Imai et al. (loc. cit.) are summarized in Table 4 below. Negative controls showed no inhibitory effect on the tested pathogen strains (data not shown) thereby confirming that antimicrobial activity is attributable to the tested compounds.

TABLE 4
Antimicrobial activity of darobactin A and darobactin derivative 9 produced in accordance with
the method of the invention compared to darobactin A activity data published in Imai et al.

| | K. pneumoniae | P. aeruginosa | E. coli | A. baumannii |
|---|---|---|---|---|
| | DSM-30104 | PA01 | ATCC25922 | DSM-30008 |
| MIC [μg/ml] of darobactin A | 4 | 4 | 2 | 4 |
| (produced by method of invention) | | | | |
| MIC [μg/ml] of darobactin D9 | 8 | 0.5 | 2 | 0.5 |
| (produced by method of invention) | | | | |
| MIC [μg/ml] of darobactin A | 4 | 2 | 2 | 4 |
| (Imai et al. 2019) | | | | |

As demonstrated above, darobactin A produced by the method of the invention has at least the same antimicrobial activity against Gram-negative bacteria as reported by Imai et al. Consequently, the production method of the invention provides a viable and efficient way to produce active compounds of interest selectively in high yields. Moreover, the novel darobactin D9 has comparable or even higher activity against the tested Gram-negative bacteria than Darobactin A.

### Assessment of Antimicrobial Activity of Darobactin Derivatives

Crude extracts of darobactin derivatives obtained by the heterologous expression and separation protocol described above were tested for their activity against exemplified Gram-negative bacteria with a minimum inhibitory concentration (MIC) assay. The presence of darobactin derivatives in the crude extracts was verified by checking the expected masses via LC-MS.

For the MIC assay, 10 μl of crude extract of each test sample, diluted in 50% methanol/H₂O, was added each into a well of the first row of a 96 well plate. After evaporation of the methanol/H₂O solvent, until only about 4 μl solvent was remaining in each well of the first row, 71 μl cation-adjusted Mueller-Hinton broth medium was added to each of said wells, so that each well in the first row of the 96 well plate had a volume of 75 μl. Then, 75 μl of cation-adjusted Mueller-Hinton broth medium was added to each well and the samples contained in the first row were mixed with the 75 μl medium/extract mixture. Starting from the 150 μl mixtures in the first row, serial dilutions of the respective mixtures were prepared in corresponding subjacent rows of the 96 well plate. Bacterial suspensions of each strain to be tested were diluted 1:100 in cation-adjusted Mueller-Hinton broth to obtain a final inoculum of approximately 10⁶ CFU/mL. 75 μl of each bacterial suspension to be tested was added to the respective crude extract samples in the wells of the 96 well plate and the plates were incubated at either 30° C. or 37° C. for at least 24 h under static conditions. Plates were then checked visually and MIC was determined as lowest concentration where no visible growth was observed. In order to quantify the activity of the obtained darobactin derivatives, their relative activity in relation to the activity of darobactin A was determined. The amount of darobactin derivative present in the extract test sample was calculated based on the area under the curve (AUC) of the mass peak as automatically calculated by software DataAnalysis 4.2 and extracted from the LC-MS measurements of each of the crude extract samples. Using the AUC of the mass peak of darobactin A (DA) as calibration standard, i.e. ratio between AUC of darobactin derivative and darobactin A was correlated to amount of compound present in sample. The results of activities of tested darobactin derivatives against exemplified Gram-negative bacteria as compared to the activity of darobactin A are summarized in Table 5 below. A value of ‘+++’ indicates that the derivative has the same antimicrobial activity as darobactin A, a value of 0 (no activity) to ‘++’ indicates a lower activity than darobactin A, and a value of ‘++++’ to ‘++++++’ indicates a higher activity than darobactin A.

TABLE 5
Relative activity of tested compounds against exemplified Gram-negative bacteria

| Darobactin / | Klebsiella | Pseudomonas | | Acinetobacter |
|---|---|---|---|---|
| Darobactin | pneumoniae | aeruginosa | E. coli | baumannii |
| Derivative | DSM-30104 | PA01 | ATCC25922 | DSM-30008 |
| A | +++ | +++ | +++ | +++ |
| B | ++++++ | ++++++ | ++++++ | ++++++ |
| B* | ++++++ | ++++++ | ++++++ | ++++++ |
| D | 0 | ++++ | ++++ | ++++ |
| 2 | 0 | ++++ | ++++ | ++++ |
| 2* | 0 | ++++ | ++++ | ++++ |
| 4 | ++ | +++ | +++ | +++ |
| 4* | ++ | +++ | +++ | +++ |
| 6 | ++++ | +++++ | +++++ | +++++ |
| 8 | 0 | ++++++ | ++++++ | ++++++ |
| 9 | +++ | +++ | ++++ | ++++ |
| 10 | 0 | + | 0 | 0 |
| 11 | ++++ | +++++ | +++++ | +++++ |
| 12 | 0 | +++++ | +++++ | +++++ |
| 12* | 0 | +++++ | +++++ | +++++ |
| 14 | +++ | ++ | +++ | ++ |
| 16 | + | + | + | + |

As demonstrated above, darobactin derivatives have an excellent antimicrobial activity against Gram-negative bacteria, and some show selectivity against certain pathogens.

### Artificial Darobactin 9 (D9) Derivatives

For the generation of new artificial darobactin 9 (D9) derivatives with altered amino acid sequences, the propeptide sequences in the darA gene were modified in silico by exchange of certain nucleotides and/or codons. Artificial darA gene fragments containing the modified propetide sequences were prepared by overlap extension polymerase chain reaction (OE-PCR) using plasmid expression vector pNOSOdarA-E-D9 (DSM 33802), which harbours the propeptide sequence of D9 (i.e. WNWSKSW), as template. To insert the required mutations into the propeptide sequence, specific reverse primers (denoted as pr2darXX_rv with XX indicating the number of the designated darobactin derivative; e.g., pr2dar22_rv includes substitutions (mismatches compared to template sequence) required for the DNA propeptide core sequence of darobactin derivative 22), which included the substitutions and a complementary sequence with primer pr3dartrp_fw at their 5′ end, were designed. A first DNA fragment was generated using primer pr1dar_fw and one of pr2darXX_rv. Primer pr1dar_fw binds to a region located in the T7 lac promoter of the plasmid expression vector pNOSOdarA-E-D9 (DSM 33802), and primer pr2darXX_rv binds to the core region of darA, contains the required substitutions encoding the desired darobactin derivative and carries a region of 13 base pairs that overlaps with primer pr3dartrp_fw. A second DNA fragment was generated using primers pr3dartrp_fw and pr4dar_rv. Primer pr4dar_rv binds in front of the termination region at the end of the intergenic region between darA and darB of the plasmid expression vector pNOSOdarA-E-D9, and primer pr3dartrp_fw binds to the core region of darA. The respective first DNA fragment (containing the mutations) was then combined with the second DNA fragment, and a third PCR reaction with primers pr1dar_fw and pr4dar_rv was performed to thereby generate a fusion fragment, due to PCR based overlap extension, harbouring the modified darA propeptide core sequence and unique restriction sites on the 3′ and 5′ end, respectively. Primer sequences are listed in Table 6; substitutions are shown in bold.

TABLE 6
Oligonucleotide primers used for OE-PCR

| Primer | Sequence |
|---|---|
| pr1dar_fw | GGTGATGTCGGCGATATAGG |
| pr3dartrp_fw | TGGCAGGAAATTTAAAGCTTATCCCAT |
| pr4dar_rv | TGGGGATCCTCAGGACTGCAG |
| pr2dar22_rv | AAATTTCCTGCCACCGTTTAGTCCAGTTCC |
| pr2dar23_rv | AAATTTCCTGCCAATGTTTAGTCCAGTTCC |
| pr2dar24_rv | AAATTTCCTGCCATGATTTAGTCCAGTTCC |
| pr2dar25_rv | AAATTTCCTGCCACGCTTTAGTCCAGTTCC |
| pr2dar30_rv | AAATTTCCTGCCATGATTTCGCCCAGTTCC |
| pr2dar31_rv | AAATTTCCTGCCACCGTTTCGCCCAGTTCC |
| pr2dar32_rv | AAATTTCCTGCCACCGTTTACACCAGTTCC |
| pr2dar33_rv | AAATTTCCTGCCATGATTTACACCAGTTCC |
| pr2dar34_rv | AAATTTCCTGCCACGCTTTACACCAGTTCC |
| pr2dar36_rv | AAATTTCCTGCCATTTTTTAGTCCAGTTCC |
| pr2dar37_rv | AAATTTCCTGCCAAGTTTTTGACCAGTTCC |
| pr2dar38_rv | AAATTTCCTGCCAAGTTTTAGTCCAGTTCC |
| pr2dar39_rv | AAATTTCCTGCCACCGCCGAGTCCAGTTCCA |

The product of the ‘overlap extension’ PCR reaction was gel-purified and the obtained DNA fragment was cloned into plasmid pNOSOdarA-E-D9 by replacing the sequence encoding darobactin derivative D9 with the modified darA gene fragment using restriction/ligation-based cloning techniques. Using this strategy, plasmid expression vectors with altered propeptide sequences in the darA gene were generated (e.g. pNOSOdarA-E-D22 to pNOSOdarA-E-D25, pNOSOdarA-E-D30 to pNOSOdarA-E-D34 and pNOSOdarA-E-D36 to pNOSOdarA-E-D39). Table 7 shows modified propeptide DNA sequences of modified darA gene fragments and their corresponding propetide amino acid core sequence. The nucleotide substitutions compared to D9-encoding darA core sequence are shown in bold and the designation of the plasmid expression vectors corresponds to the nomenclature of the darobactin derivatives, e.g., pNOSOdarA-E-D22 harbours the propeptide sequence of darobactin 22 (D22). Correctness of the plasmid sequences was verified by restriction hydrolysis followed by agarose gel electrophoresis and sanger sequencing at LGS genomics.

TABLE 7
Propeptide DNA and protein sequences
of darobactin derivatives.

| | | Propeptide |
|---|---|---|
| Darobactin | DNA propeptide | amino acid |
| derivative | core sequence | sequence |
| 22 | TGG AAC TGG ACT AAA CGG TGG | WNWTKRW |
| 23 | TGG AAC TGG ACT AAA CAT TGG | WNWTKHW |
| 24 | TGG AAC TGG ACT AAA TCA TGG | WNWTKSW |
| 25 | TGG AAC TGG ACT AAA GCG TGG | WNWTKAW |
| 30 | TGG AAC TGG GCG AAA TCA TGG | WNWAKSW |
| 31 | TGG AAC TGG GCG AAA CGG TGG | WNWAKRW |
| 32 | TGG AAC TGG TGT AAA CGG TGG | WNWCKRW |
| 33 | TGG AAC TGG TGT AAA TCA TGG | WNWCKSW |
| 34 | TGG AAC TGG TGT AAA GCG TGG | WNWCKAW |
| 36 | TGG AAC TGG ACT AAA AAA TGG | WNWTKKW |
| 37 | TGG AAC TGG TCA AAA ACT TGG | WNWSKTW |
| 38 | TGG AAC TGG ACT AAA ACT TGG | WNWTKTW |
| 39 | TGG AAC TGG ACT CGG CGG TGG | WNWTRRW |

### Heterologous Expression and Production

For heterologous production of the darobactin derivatives, one of the plasmid expression vectors described above, i.e. pNOSOdarA-E-D22 to pNOSOdarA-E-D25, pNOSOdarA-E-D30 to pNOSOdarA-E-D34 and pNOSOdarA-E-D36 to pNOSOdarA-E-D39, were separately transformed into E. coli BL21 (DE3) cells by electroporation, and the respective recombinant strains were cultivated to investigate their darobactin production profile as described below.

### Fermentation/Cultivation of Host Cells

### Day 1

Inoculation of a single clone of E. coli BL21 (DE3) cells harbouring plasmid pNOSOdarA-E with modified darA gene in 10 ml LB medium including 30 μg/ml Kanamycin (Kan30) and incubation overnight at 30° C., 180 rpm (Infors HT)

### Day 2

Inoculation of 50 ml FM medium (1.254% K2HPO4, 0.231% KH2PO4, 0.4% D(+)-glucose, 0.1% NH₄Cl, 1.2% yeast extract, 0.5% NaCl and 0.0492% MgSO4(*7H2O), pH 7.1) including Kan30 and 1 mg/l VitaminB12 with 1% (v/v) of preculture
Incubation for 3 days at 30° C., 180 rpm (Infors HT)

### Separation and Retaining of Compounds

### Day 5

Harvest supernatant by centrifugation at 8,000 g for 10 min at 4° C.
Incubation of supernatant with 0.05% XAD16N resin (Sigma Aldrich) (w/v) under agitation for 2 h
Harvest of XAD16N resin and elution of bound compounds from XAD16N resin with 10 ml 80% Methanol under shaking for 1.5 h
Drying of eluate in a rotary evaporator
Solubilization of evaporated extract in 0.5 ml 50/50 methanol/water mixture; or in 0.5 ml 100% water
HPLC-MS analysis to verify the production of peptide compound of interest, e.g., darobactin A or darobactin derivative; characteristic ions for darobactins in tandem MS mode are m/z 160.075 and a prominent [M-H₂O+2H]²⁺ ion.

In case of production of compounds for antimicrobial testing with the minimum inhibitory concentration assays described below, no antibiotic was added to the expression cultures as a selection marker.

HPLC purification was carried out on a Waters Autopurifier (Eschborn, Germany) high pressure gradient system, equipped with 2545 binary gradient module, SFO system fluidics organizer, 2767 sample manager and a 2998 photodiode array detector coupled to a 3100 single quadrupole mass spectrometer operated in positive ion mode. Source and voltage settings for the MS were as follows: mass range, m/z 300-1000; scan duration, 1 s; points per Dalton, 4; capillary voltage, 3.5 kV; cone voltage, 30 V; extractor voltage 3 V; RF lens, 0.1 V; source temperature 120° C., desolvation temperature, 250° C.; desolvation gas flow, 400 L/h; cone gas flow, 50 L/h; ion counting threshold, 30. Separation was carried out on a Waters X-Bridge prep C-18 5 μm ODB, 150×19 mm column using ACN+0.1% FA as B and H₂O+0.1% FA as A and a flow rate of 25 ml/min. Compound detection was carried out by detection of the [M+2H]²⁺. Second purification step was carried out on the same instrument on an Phenomenex Kinetex 5u Biphenyl 100A 250×21.2 mm using ACN+0.1% FA as B and H₂O+0.1% FA as A and a flow rate of 25 ml/min. Where necessary, a Dionex Ultimate 3000 SDLC low pressure gradient system on a Waters CSH Phenyl-Hexyl 250 mm×10 mm 5 μm dp column with the eluents H₂O+0.1% FA as A and ACN+0.1% FA as B, a flow rate of 5 ml/min and a column thermostated at 30° C. was used for further purification of peptide compounds of interest. Peptide compounds of interest, e.g. darobactin A and darobactin derivatives, can be detected by UV absorption at 254 nm.

Following the above protocol, darobactin 22 was, for example, heterologously produced by expression of a modified artificial plasmid expression vector pNOSOdarA-E-D22 comprising propeptide sequence D22 (see, Table 2 above) in E. coli BL21(DE3) cells.

### Example Compounds

Exemplified compounds obtained by heterologous expression of the modified BGCs according to the present invention are shown in Tables 8 and 9 below. It was found that darobactin derivatives with sulfur-containing amino acids carry a protection group (proved via Nuclear Magnetic Resonance (NMR) spectroscopy).

TABLE 8
Produced example compounds D22-D25*, D30, D31, and D36-D39

| | | Observed |
|---|---|---|
| Darobactin | Chemical structure based on amino acid sequence of core | mass* (m/z) |
| derivative | peptide, mass and MS2 fragmentation pattern | [M + 2H]⁺² |
| D22 | 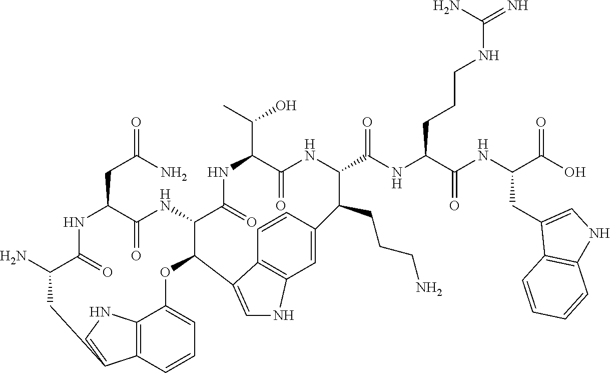 [SMILES: C[C@H](O)[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CCCNC(=N)N)C(=O)N[C@@H](Cc2c[nH]c3ccccc23)C(=O)O)NC1=O] | 544.76 |
| D22* | 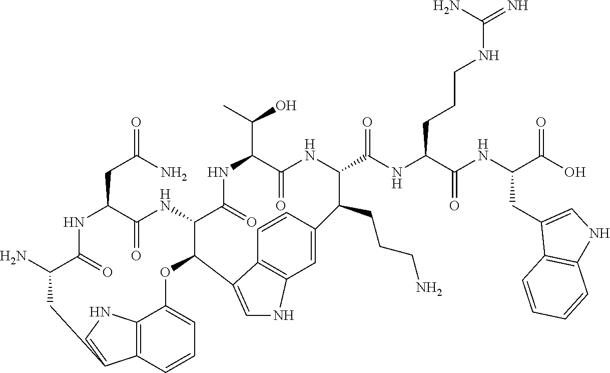 [SMILES: C[C@@H](O)[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CCCNC(=N)N)C(=O)N[C@@H](Cc2c[nH]c3ccccc23)C(=O)O)NC1=O] | 544.76 |
| D23 | 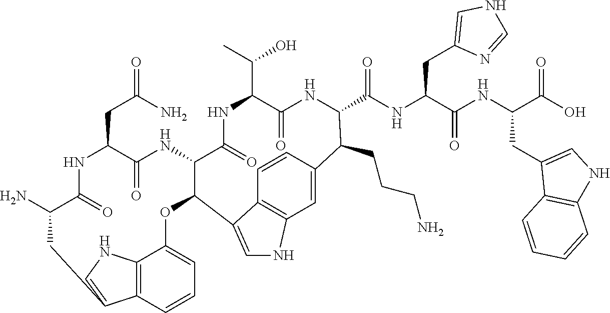 [SMILES: C[C@H](O)[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](Cc2c[nH]cn2)C(=O)N[C@@H](Cc2c[nH]c3ccccc23)C(=O)O)NC1=O] | 535.23 |
| D23* | 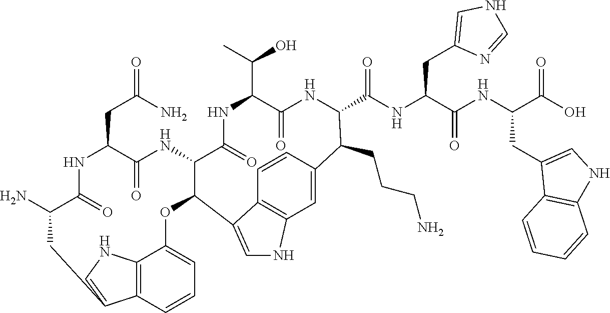 [SMILES: C[C@@H](O)[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](Cc2c[nH]cn2)C(=O)N[C@@H](Cc2c[nH]c3ccccc23)C(=O)O)NC1=O] | 535.23 |
| D24 | 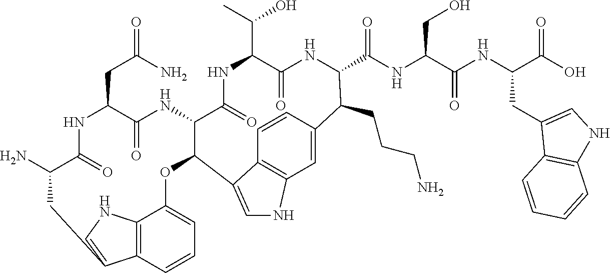 [SMILES: C[C@H](O)[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CO)C(=O)N[C@@H](Cc2c[nH]c3ccccc23)C(=O)O)NC1=O] | 510.22 |
| D24* | 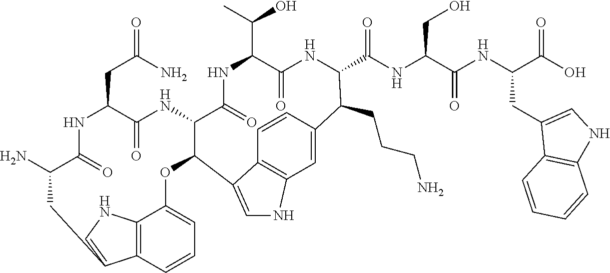 [SMILES: C[C@@H](O)[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CO)C(=O)N[C@@H](Cc2c[nH]c3ccccc23)C(=O)O)NC1=O] | 510.22 |
| D25 | 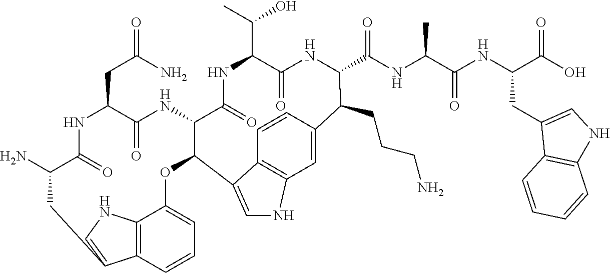 [SMILES: C[C@H](NC(=O)[C@H]1NC(=O)[C@H]([C@H](C)O)NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@@H]1CCCN)C(=O)N[C@@H](Cc1c[nH]c2ccccc12)C(=O)O] | 502.22 |
| D25* | 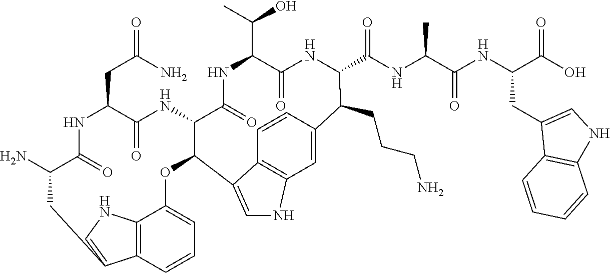 [SMILES: C[C@H](NC(=O)[C@H]1NC(=O)[C@H]([C@@H](C)O)NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@@H]1CCCN)C(=O)N[C@@H](Cc1c[nH]c2ccccc12)C(=O)O] | 502.22 |
| D30 | 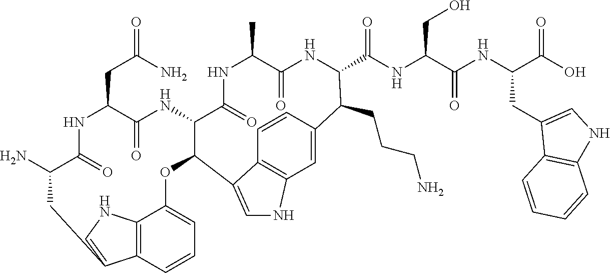 [SMILES: C[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CO)C(=O)N[C@@H](Cc2c[nH]c3ccccc23)C(=O)O)NC1=O] | 495.21 |
| D31 | 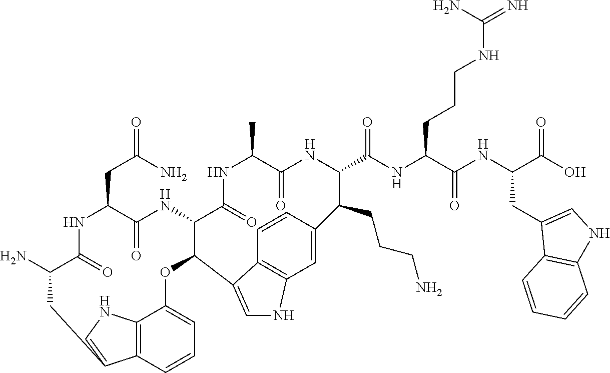 [SMILES: C[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CCCNC(=N)N)C(=O)N[C@@H](Cc2c[nH]c3ccccc23)C(=O)O)NC1=O] | 529.75 |
| D36 | 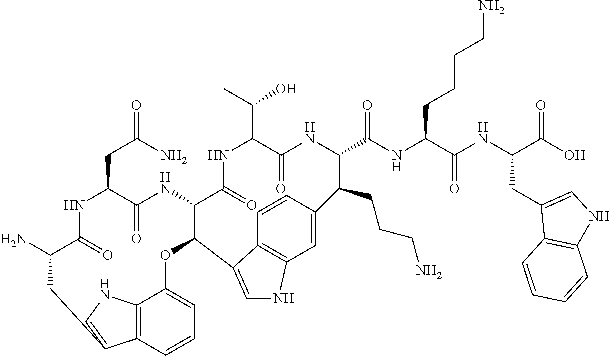 [SMILES: C[C@H](O)C1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CCCCN)C(=O)N[C@@H](Cc2c[nH]c3ccccc23)C(=O)O)NC1=O] | 530.75 |
| D36* | 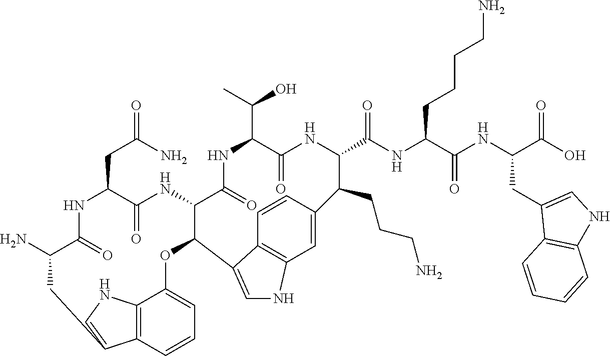 [SMILES: C[C@@H](O)[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CCCCN)C(=O)N[C@@H](Cc2c[nH]c3ccccc23)C(=O)O)NC1=O] | 530.75 |
| D37 | 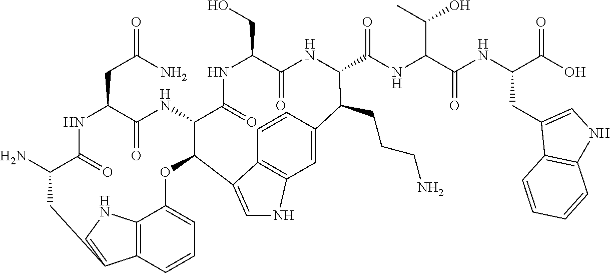 [SMILES: C[C@H](O)C(NC(=O)[C@H]1NC(=O)[C@H](CO)NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@@H]1CCCN)C(=O)N[C@@H](Cc1c[nH]c2ccccc12)C(=O)O] | 510.22 |
| D37* | 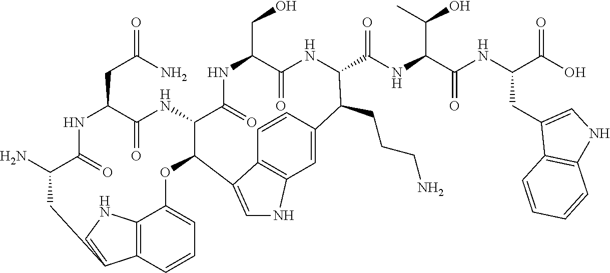 | 510.22 |
| D38 | 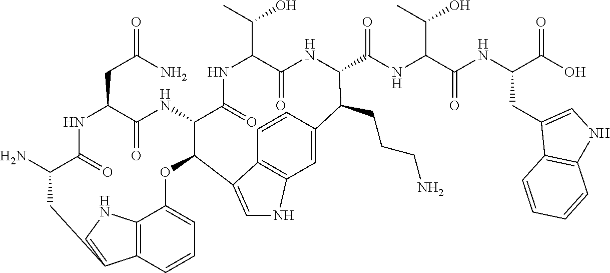 [SMILES: C[C@H](O)C1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)NC(C(=O)N[C@@H](Cc2c[nH]c3ccccc23)C(=O)O)[C@H](C)O)NC1=O] | 517.22 |
| D38* | 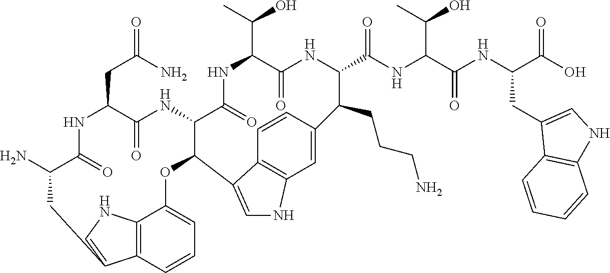 [SMILES: C[C@@H](O)C(NC(=O)[C@H]1NC(=O)[C@H]([C@@H](C)O)NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@@H]1CCCN)C(=O)N[C@@H](Cc1c[nH]c2ccccc12)C(=O)O] | 517.22 |
| D39 | 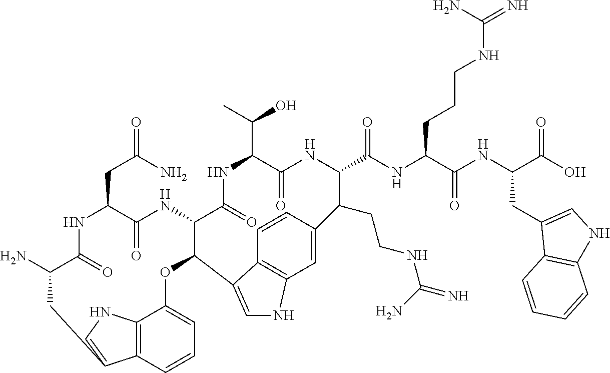 [SMILES: C[C@@H](O)[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)C(CCNC(=N)N)[C@@H](C(=O)N[C@@H](CCCNC(=N)N)C(=O)N[C@@H](Cc2c[nH]c3ccccc23)C(=O)O)NC1=O] | 558.75 |

*All observed [M + 2H]²⁺ masses correlate with the expected [M + 2H]²⁺ masses.

TABLE 9A
Produced example compounds PD32, PD33 and PD34 and PD32*,
PD33* and PD34* with sulfur-containing amino acid

| | | Observed |
|---|---|---|
| Darobactin | Chemical structure based on amino acid sequence of core | mass* (m/z) |
| derivative | peptide, mass, MS2 fragmentation pattern and NMR | [M + 2H]²⁺ |
| PD32 | 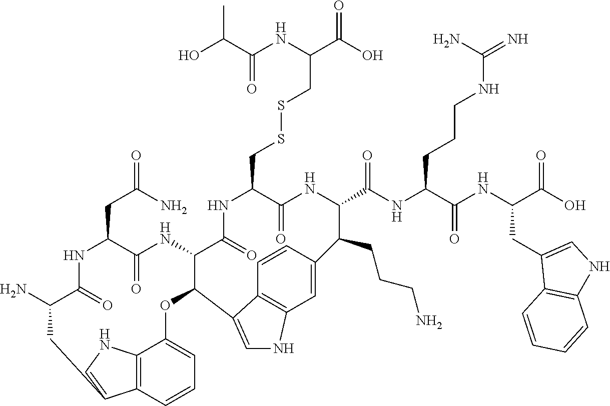 [SMILES: CC(O)C(=O)NC(CSSC[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CCCNC(=N)N)C(=O)N[C@@H](Cc2c[nH]c3ccccc23)C(=O)O)NC1=O)C(=O)O] | 641.25 |
| PD32* | 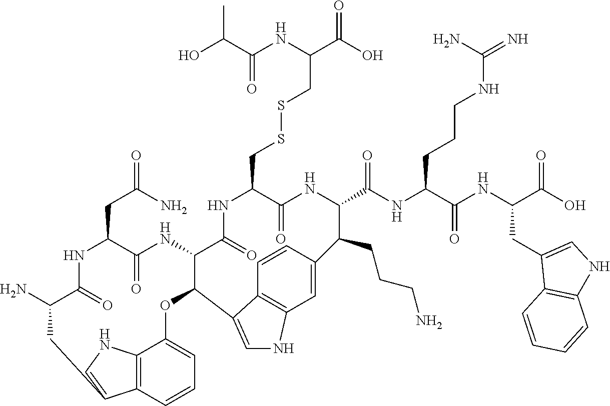 [SMILES: CC(O)C(=O)NC(CSSC[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CCCNC(=N)N)C(=O)N[C@@H](Cc2c[nH]c3ccccc23)C(=O)O)NC1=O)C(=O)O] | 641.25 |
| PD33 | 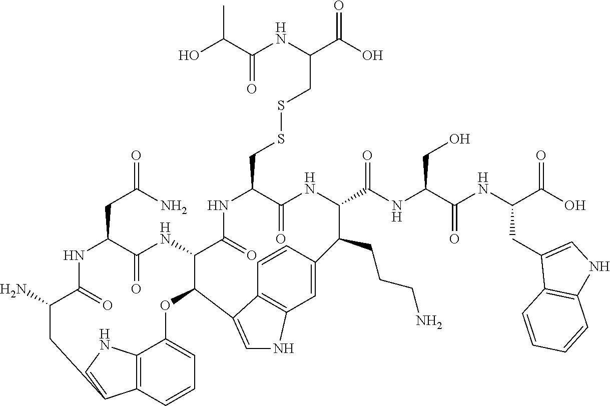 [SMILES: CC(O)C(=O)NC(CSSC[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CO)C(=O)N[C@@H](Cc2c[nH]c3ccccc23)C(=O)O)NC1=O)C(=O)O] | 606.72 |
| PD33* | 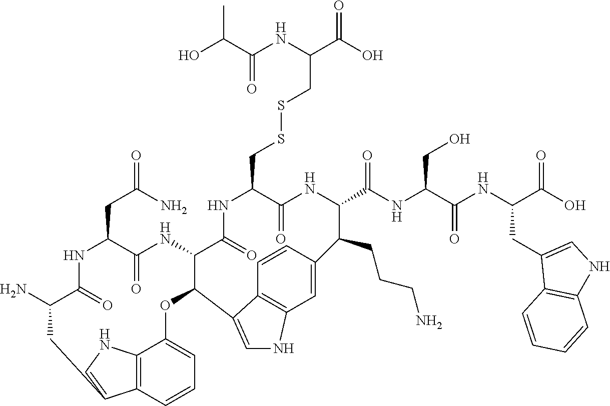 [SMILES: CC(O)C(=O)NC(CSSC[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](CO)C(=O)N[C@@H](Cc2c[nH]c3ccccc23)C(=O)O)NC1=O)C(=O)O] | 606.72 |
| PD34 | 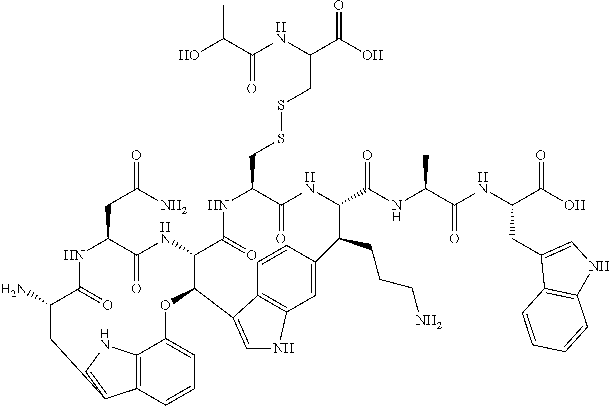 [SMILES: CC(O)C(=O)NC(CSSC[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](C)C(=O)N[C@@H](Cc2c[nH]c3ccccc23)C(=O)O)NC1=O)C(=O)O] | 598.72 |
| PD34* | 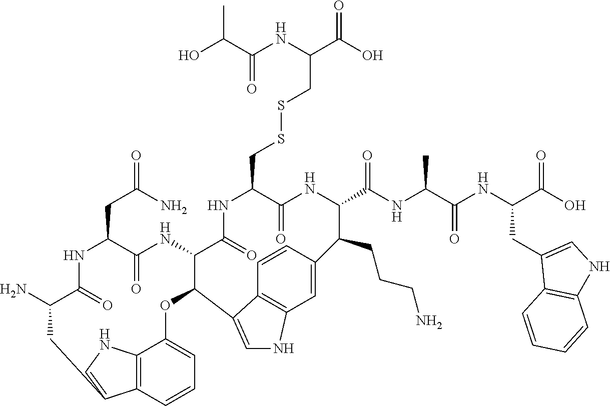 [SMILES: CC(O)C(=O)NC(CSSC[C@@H]1NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@H](CCCN)[C@@H](C(=O)N[C@@H](C)C(=O)N[C@@H](Cc2c[nH]c3ccccc23)C(=O)O)NC1=O)C(=O)O] | 598.72 |

Darobactins D32 to D34 and D32* to D34* shown in Table 9B can be prepared from the protected compounds PD32 to PD34* by following standard protocols for the cleavage of disulfide bonds using dithiobutylamine (DTBA), tris (2-carboxyethyl) phosphine hydrochloride (TCEP-HCl) or mercaptoethanole or dithiothreitol (DTT). Such standard procedures are generally known in the art, and have, for example, been described by Kirley, Terence L. et al. (2016), Selective disulfide reduction for labeling and enhancement of Fab antibody fragments, Biochemical and Biophysical Research Communications 480 (4), pp. 752-757 (DOI: 10.1016/j.bbrc.2016.10.128)-Lukesh, John C. et al. (2012), A Potent, Versatile Disulfide-Reducing Agent from Aspartic Acid, Journal of the American Chemical Society 134 (9), pp. 4057-4059 (DOI: 10.1021/ja211931f); and Yu, Shukun et al. (2020), Comparative study of protease hydrolysis reaction demonstrating Normalized Peptide Bond Cleavage Frequency and Protease Substrate Broadness Index, PLoS ONE 15 (9), e0239080 (DOI: 10.1371/journal.pone.0239080)

TABLE 9B
Compounds D32-D34 and D32*-D34*

| | | Observed |
|---|---|---|
| Darobactin | Chemical structure based on amino acid sequence of core | mass* (m/z) |
| derivative | peptide, mass and MS2 fragmentation pattern | [M + 2H]²⁺ |
| D32 | 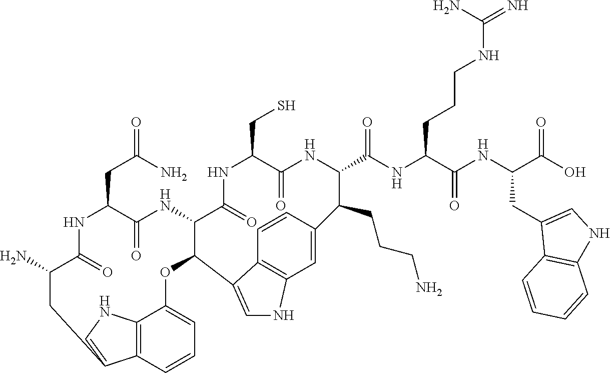 [SMILES: N=C(N)NCCC[C@H](NC(=O)[C@H]1NC(=O)[C@H](CS)NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@@H]1CCCN)C(=O)N[C@@H](Cc1c[nH]c2ccccc12)C(=O)O] | 545.73 |
| D32* | 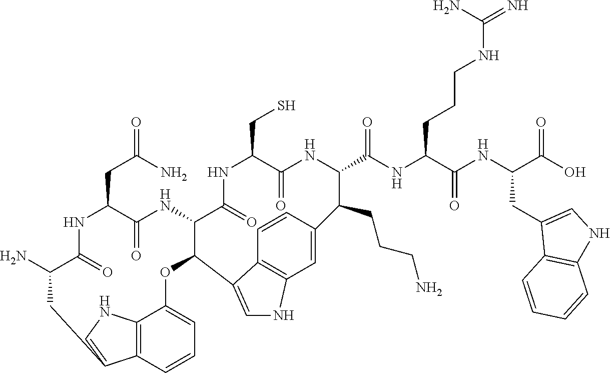 [SMILES: N=C(N)NCCC[C@H](NC(=O)[C@H]1NC(=O)[C@H](CS)NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@@H]1CCCN)C(=O)N[C@@H](Cc1c[nH]c2ccccc12)C(=O)O] | 545.73 |
| D33 | 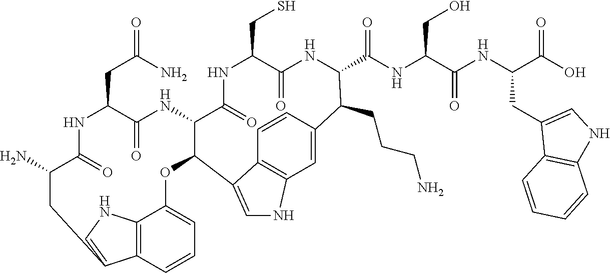 [SMILES: NCCC[C@H]1c2ccc3c(c[nH]c3c2)[C@H]2Oc3cccc4c(c[nH]c34)C[C@H](N)C(=O)N[C@@H](CC(N)=O)C(=O)N[C@@H]2C(=O)N[C@@H](CS)C(=O)N[C@@H]1C(=O)N[C@@H](CO)C(=O)N[C@@H](Cc1c[nH]c2ccccc12)C(=O)O] | 511.20 |
| D33* | 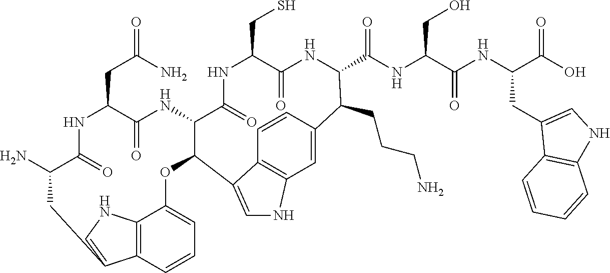 [SMILES: NCCC[C@H]1c2ccc3c(c[nH]c3c2)[C@H]2Oc3cccc4c(c[nH]c34)C[C@H](N)C(=O)N[C@@H](CC(N)=O)C(=O)N[C@@H]2C(=O)N[C@@H](CS)C(=O)N[C@@H]1C(=O)N[C@@H](CO)C(=O)N[C@@H](Cc1c[nH]c2ccccc12)C(=O)O] | 511.20 |
| D34 | 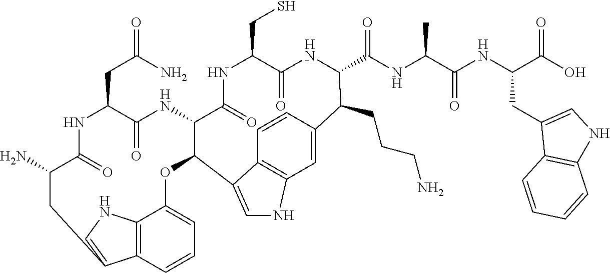 [SMILES: C[C@H](NC(=O)[C@H]1NC(=O)[C@H](CS)NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@@H]1CCCN)C(=O)N[C@@H](Cc1c[nH]c2ccccc12)C(=O)O] | 503.20 |
| D34* | 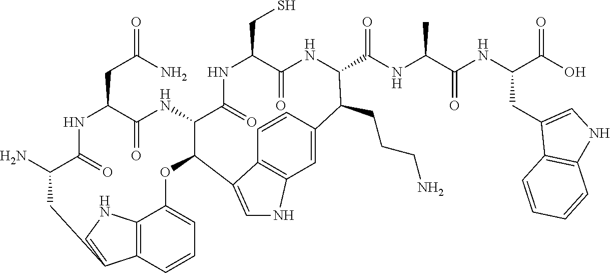 [SMILES: C[C@H](NC(=O)[C@H]1NC(=O)[C@H](CS)NC(=O)[C@H]2NC(=O)[C@H](CC(N)=O)NC(=O)[C@@H](N)Cc3c[nH]c4c(cccc34)O[C@@H]2c2c[nH]c3cc(ccc23)[C@@H]1CCCN)C(=O)N[C@@H](Cc1c[nH]c2ccccc12)C(=O)O] | 503.20 |

### Generation of Halogenated Darobactin Derivatives by Feeding Halogenated Tryptophan During Heterologous Expression and Production

For heterologous production of halogenated darobactin derivatives, for example commercially available 5-chloro-L-tryptophan, 7-chloro-L-tryptophan, 6-fluoro-L-tryptophan or 7-fluor-L-tryptophan can be fed periodically in different concentrations (total end concentration can be for example between 0.5 mM and 10 mM) to the fermentation broth during cultivation of E. coli BL21-Gold (DE3) (Agilent Technologies). E. coli was previously transformed with pNOSOdarA-E-DX, where X stands for any generated expression plasmid. The cultivation and feeding periods were performed as described below:

### Fermentation/Cultivation of Host Cells

### Day 1

Inoculation of a single clone of E. coli BL21-Gold (DE3) (Agilent Technologies) cells harbouring plasmid pNOSOdarA-E-DX with modified darA gene, in 10 ml LB medium including 30 μg/ml Kanamycin (Kan30) and incubation overnight at 30° C., 180 rpm (Infors HT)

### Day 2

Inoculation of 20 ml FM medium (1.254% K2HPO4, 0.231% KH2PO4, 0.4% D(+)-glucose, 0.1% NH₄Cl, 1.2% yeast extract, 0.5% NaCl and 0.0492% MgSO4(*7H2O), pH 7.1) including Kan30 and 1 mg/l VitaminB12 with 1% (v/v) of preculture
Preparation of halogenated tryptophan stocks by dissolving appropriate mass in dH₂O. For example: Dissolving of 10 mg 6-fluoro-L-tryptophan in 1.2 ml dH₂O to reach an end concentration of approximately 2.1 mM in 20 mL of production culture and storage of the solution at 4° C.
Addition of 0.4 μM IPTG (end concentration) to induce T7/ac promoter at OD₆₀₀ of 0.8 to 1.2 and first feeding of 1/6 (v/v) of prepared halogenated tryptohan stock
2 h after first feeding, second feeding with 1/6 (v/v) of prepared halogenated tryptophan stock

### Day 3

18 h after last feeding, 1/6 (v/v) of prepared halogenated tryptophan stock was fed
6 h after last feeding, 1/6 (v/v) of prepared halogenated tryptophan stock was fed

### Day 4

18 h after last feeding, 1/6 (v/v) of prepared halogenated tryptophan stock was fed
6 h after last feeding, 1/6 (v/v) of prepared halogenated tryptophan stock was fed to reach total concentration of for example 0.5 mM to 10 mM, depending of chosen stock concentration.
Complete cultivation performed at 30° C. and 180 rpm (Infors HT)

Alternative for use of commercially available halogenated tryptophan for feeding to generate halogenated darobactin is the use of E. coli pUC18-zeo-mx8-corP-trpAB to produce halogenated tryptophan out of serine and halogenated indole. Vector pUC18-zeo-mx8-corP-trpAB was generated out of pUC18-zeo-mx8-corP (Pogorevc, D. et al. Production optimization and biosynthesis revision of corallopyronin A, a potent anti-filarial antibiotic. Metab. Eng. 55, 201-211; 10.1016/j.ymben.2019.07.010 (2019)) and trpAB gene of pSTB7 37845 (ATCC) after restriction digestion with NsiI and NdeI and followed ligation according to standard molecular biology techniques. Co-expression of pNOSOdarA-E-DX and pUC18-zeo-mx8-corP-trpAB in E. coli BL21-Gold (DE3) (Agilent Technologies) lead to production of halogenated tryptophan through feeding of for example 10 mM serine and 5 to 10 mM desired indole. This system is flexible and the trpAB gene can also be cloned upstream darE into the BGC of Darobactin via unique restriction sites SpeI and PacI (R₁₁ and R₁₂, shown in Table 1). The trpAB gene, encodes tryptophan synthase enzymes TrpA and TrpB, catalyzing generation of tryptophan out of serine and indole. This allows modifications of darobactin with all commercially available or synthetically generated indoles that can be incorporated into darobactin during translation.

### Separation and Retaining of Compounds

### Day 5

In theory, after feeding with halogenated tryptophan, the appropriate halogenated tryptophan could be incorporated at each position where a tryptophan is assumed according to genetic code. For example, derivatives of Darobactin 9 with a 6-fluoro-L-tryptophan can carry the 6-fluoro-L-tryptophan at position 1, 3 or 7, at position 1 and 3, 1 and 7, 3 and 7, or at position 1, 3 and 7 of the respective core peptide amino acid (see FIG. 2). The naming of these halogenated compounds follows the pattern “darobactin derivative” (e.g. darobactin 9)-“position of fluorine on tryptophan according to IUPAC” (e.g. 6F for 6-fluoro-L-tryptophan”), followed by the position of the halogenated tryptophan in the amino acid of the core peptide (e.g. 1, 3 or 7). As an example, darobactin 9-6F1 denotes Darobactin 9 with a 6-fluoro-L-tryptophan at position 1. In case of multiple halogenated tryptophans, positions are separated by hyphenation, e.g. darobactin 9-6F1-7 denoting Darobactin 9 with a 6-fluoro-L-tryptophan at positions 1 and 7. To check which new derivatives were produced, compounds were extracted and analyzed with HPLC-MS and HPLC-MS-MS detailed described below:
Harvest supernatant by centrifugation at 8,000 g for 10 min at 4° C.
Incubation of supernatant with 2 ml XAD16N resin (Sigma Aldrich) (w/v) under agitation for 2 h
Harvest of XAD16N resin and elution of bound compounds from XAD16N resin with 10 ml 80% Methanol under shaking for 1.5 h
Drying of eluate in a rotary evaporator
Solubilization of evaporated extract in 0.2 ml 50/50 methanol/water mixture; or in 0.2 ml 100% water
HPLC-MS analysis to verify the production of peptide compound of interest, e.g., darobactin 9-F1 or darobactin derivative; characteristic ions for darobactins in tandem MS mode are m/z 160.075 and a prominent [M-H₂O+2H]²⁺ ion.

As an example, production of darobactin 9-6F derivatives was performed according to described protocol by feeding 6-fluoro-L-tryptohan during heterologous expression and production. According to HPLC-MS and MS-MS analysis, darobactin 9-6F1, darobactin 9-6F7 and darobactin 9-6F1-7 were produced in high amounts. In some embodiments, darobactin 9-6F1 and 9-6F7 were preferably produced, especially darobactin 9-6F1. Further examples for the production of halogenated darobactin derivatives described herein are darobactin 9-7F1, darobactin 9-7F7, darobactin 9-5CI1 and 9-5CI7. Confirmation of production was achieved by mass, isotope pattern and MS-MS fragmentation pattern, showing incorporation of halogenated tryptophans, after or before purification according to described procedure.

### Purification of Halogenated Darobactins

HPLC purification was carried out on a Waters Autopurifier (Eschborn, Germany) high pressure gradient system, equipped with 2545 binary gradient module, SFO system fluidics organizer, 2767 sample manager and a 2998 photodiode array detector coupled to a 3100 single quadrupole mass spectrometer operated in positive ion mode. Source and voltage settings for the MS were as follows: mass range, m/z 300-1000; scan duration, 1 s; points per Dalton, 4; capillary voltage, 3.5 kV; cone voltage, 30 V; extractor voltage 3 V; RF lens, 0.1 V; source temperature 120° C., desolvation temperature, 250° C.; desolvation gas flow, 400 L/h; cone gas flow, 50 L/h; ion counting threshold, 30. Separation was carried out on a Waters XBridge® Prep C-18 5 μm ODB™, 150×19 mm column using ACN+0.1% FA as B and H₂O+0.1% FA as A and a flow rate of 25 ml/min. Compound detection was carried out by detection of the [M+2H]²⁺.

Second purification step was carried out on a Thermo Scientific™ Dionex™ UltiMate™ 3000 SDLC low pressure gradient system on a Waters XSelect® Peptide CSH C18 OBD™ Prep 130 Å, 5 μm, 10 mm×250 mm column. Used eluents were H₂O+0.1% FA as A and ACN+0.1% FA as B, a flow rate of 6 ml/min and a column thermostated at 45° C. was used for further purification of peptide compounds of interest. Peptide compounds of interest, e.g. darobactin 9-6F1 can be detected by UV absorption at 254 nm or 280 nm. The two isomers darobactin 9-6F1 and darobactin 9-6F7 elute very close together, the first eluting isomer is the 6F7 variant, which is produced in less amounts, followed by darobactin 9-6F1, which is the main isomer. Exemplified compounds obtained by heterologous expression and production of the modified BGCs according to the present invention are shown in Table 10 below.

TABLE 10
Produced halogenated example compounds D9-6F1, D9-6F7, D9-6F1-
7, D9-7F7, D9-5Cl1 and D9-5Cl7

| | | Observed |
|---|---|---|
| Darobactin | Chemical structure based on amino acid sequence of core | mass* (m/z) |
| derivative | peptide, mass and MS2 fragmentation pattern | [M + 2H]²⁺ |
| D9-6F1 | 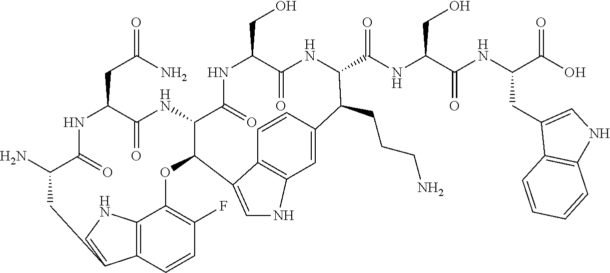 [SMILES: NCCC[C@H]1c2ccc3c(c[nH]c3c2)[C@H]2Oc3c(F)ccc4c(c[nH]c34)C[C@H](N)C(=O)N[C@@H](CC(N)=O)C(=O)N[C@@H]2C(=O)N[C@@H](CO)C(=O)N[C@@H]1C(=O)N[C@@H](CO)C(=O)N[C@@H](Cc1c[nH]c2ccccc12)C(=O)O] | 512.21 |
| D9-6F7 | 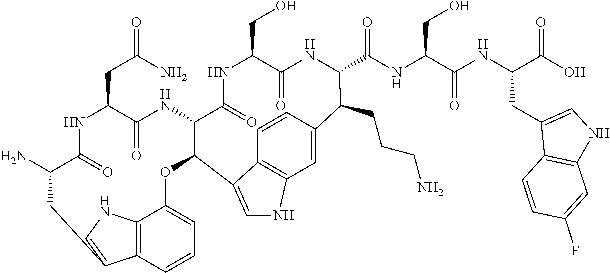 [SMILES: NCCC[C@H]1c2ccc3c(c[nH]c3c2)[C@H]2Oc3cccc4c(c[nH]c34)C[C@H](N)C(=O)N[C@@H](CC(N)=O)C(=O)N[C@@H]2C(=O)N[C@@H](CO)C(=O)N[C@@H]1C(=O)N[C@@H](CO)C(=O)N[C@@H](Cc1c[nH]c2cc(F)ccc12)C(=O)O] | 512.21 |
| D9-6F1-7 | 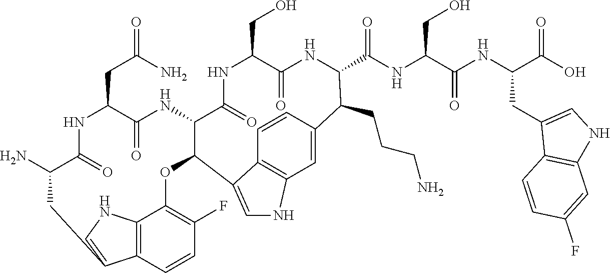 [SMILES: NCCC[C@H]1c2ccc3c(c[nH]c3c2)[C@H]2Oc3c(F)ccc4c(c[nH]c34)C[C@H](N)C(=O)N[C@@H](CC(N)=O)C(=O)N[C@@H]2C(=O)N[C@@H](CO)C(=O)N[C@@H]1C(=O)N[C@@H](CO)C(=O)N[C@@H](Cc1c[nH]c2cc(F)ccc12)C(=O)O] | 521.21 |
| D9-7F7 | 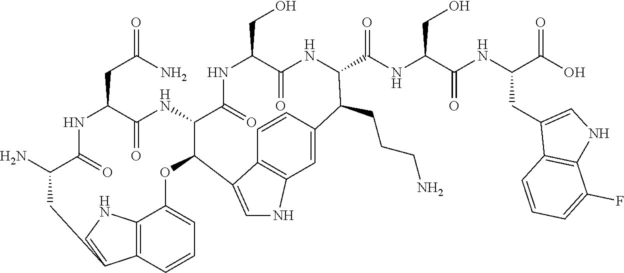 [SMILES: NCCC[C@H]1c2ccc3c(c[nH]c3c2)[C@H]2Oc3cccc4c(c[nH]c34)C[C@H](N)C(=O)N[C@@H](CC(N)=O)C(=O)N[C@@H]2C(=O)N[C@@H](CO)C(=O)N[C@@H]1C(=O)N[C@@H](CO)C(=O)N[C@@H](Cc1c[nH]c2c(F)cccc12)C(=O)O] | 512.21 |
| D9-5Cl1 | 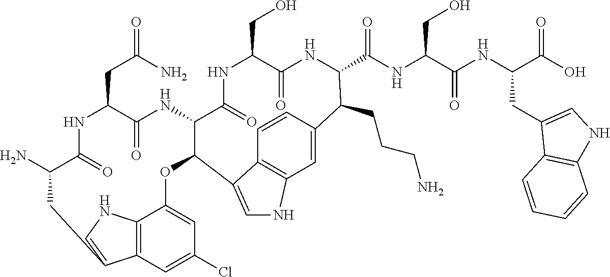 [SMILES: NCCC[C@H]1c2ccc3c(c[nH]c3c2)[C@H]2Oc3cc(Cl)cc4c(c[nH]c34)C[C@H](N)C(=O)N[C@@H](CC(N)=O)C(=O)N[C@@H]2C(=O)N[C@@H](CO)C(=O)N[C@@H]1C(=O)N[C@@H](CO)C(=O)N[C@@H](Cc1c[nH]c2ccccc12)C(=O)O] | 520.19 |
| D9-5Cl7 | 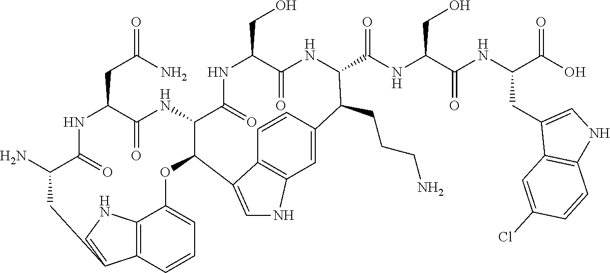 [SMILES: NCCC[C@H]1c2ccc3c(c[nH]c3c2)[C@H]2Oc3cccc4c(c[nH]c34)C[C@H](N)C(=O)N[C@@H](CC(N)=O)C(=O)N[C@@H]2C(=O)N[C@@H](CO)C(=O)N[C@@H]1C(=O)N[C@@H](CO)C(=O)N[C@@H](Cc1c[nH]c2ccc(Cl)cc12)C(=O)O] | 520.19 |

*All observed [M + 2H]²⁺ masses correlate with the expected [M + 2H]²⁺ masses.

### Assessment of Antimicrobial Activity of Darobactin Derivatives

Crude extracts containing the respective darobactin derivatives obtained by the heterologous expression and separation protocol described above were tested for their activity against exemplified Gram-negative bacteria in standard microbroth dilution assays. The presence of darobactin derivatives in the crude extracts was verified by checking the expected masses via LC-HRMS.

All microorganisms were handled according to standard procedures as recommended by the depositor. Strains were obtained from the German Collection of Cell Cultures and Microorganisms (Klebsiella pneumoniae DSM-30104, Acinetobacter baumannii DSM-30007 and A. baumannii DSM-30008), the American Type Culture Collection (Escherichia coli ATCC-25922), or were part of our internal strain collection (Pseudomonas aeruginosa PAO1, PA14 and PA14ΔmexAB).

For the MIC assay, 10 μl of crude extract of each test sample, diluted in 50% methanol/H₂O, was added each into a well of the first row of a 96 well plate. After evaporation of the methanol/H₂O solvent, until only about 4 μl solvent was remaining in each well of the first row, 71 μl cation-adjusted Mueller-Hinton broth medium was added to each of said wells, so that each well in the first row of the 96 well plate had a volume of 75 μl. Then, 75 μl of cation-adjusted Mueller-Hinton broth medium was added to each well and the samples contained in the first row were mixed with the 75 μl medium/extract mixture. Starting from the 150 μl mixtures in the first row, serial dilutions of the respective mixtures were prepared in corresponding subjacent rows of the 96 well plate. Bacterial suspensions of each strain to be tested were diluted 1:100 in cation-adjusted Mueller-Hinton broth to obtain a final inoculum of approximately 10⁶ CFU/mL. 75 μl of each bacterial suspension to be tested was added to the respective crude extract samples in the wells of the 96 well plate and the plates were incubated at either 30° C. or 37° C. for at least 24 h under static conditions. Plates were then checked visually and MIC was determined as lowest concentration where no visible growth was observed. In order to quantify the activity of the obtained darobactin derivatives, their relative activity in relation to the activity of darobactin A was determined. The amount of darobactin derivative present in the extract test sample was calculated based on the area under the curve (AUC) of the mass peak as automatically calculated by software Data Analysis 4.2 and extracted from the LC-MS measurements of each of the crude extract samples. Concentration factor of the tested extract was calculated by division of the concentration (100× caused by the extraction process from 50 ml culture to 0.5 ml extract) by the dilution factor in which visible growth of the respective strain was no longer observed. For example, if the darobactin derivative-containing crude extract in column 1 (of a standard 96 well plate) resulted in growth inhibition of the respective strain from wells A1 to E1, but not anymore in F1, the concentration factor score 0.42× was assigned to the darobactin derivative. If the darobactin derivative-containing crude extract in column 2 resulted in growth inhibition only from wells A2 to B2, but not anymore in C2, we assigned the concentration factor score 3.34×, meaning that the crude extract in column 1 showed stronger antibacterial activity (assuming that the compound concentration is similar). The results of activities of tested darobactin derivatives against exemplified Gram-negative bacteria as compared to the activity of darobactin A and D9 are summarized in Table 11 below.

TABLE 11
Activity of crude extracts of tested compounds
against exemplified Gram negative bacteria

| | Concentration factor of crude extract at | | | | AUC |
|---|---|---|---|---|---|
| Darobactin | which full growth inhibition is observed | | | | ratio |
| in crude | K. pneumoniae | P. aeruginosa | E. coli | A. baumannii | (derivative/ |
| extract | DSM-30104 | PAO1 | ATCC25922 | DMS-30008 | D9) |
| 9 | 0.42x | 0.11x | 0.42x | 0.21x | 1 |
| 22 | 0.83x | 0.05x | 0.21x | 0.05x | 0.33 |
| 22* | 0.83x | 0.05x | 0.21x | 0.05x | 0.33 |
| 23 | 0.83x | 0.11x | 0.21x | 0.21x | 0.38 |
| 23* | 0.83x | 0.11x | 0.21x | 0.21x | 0.38 |
| 24 | 0.83x | 0.05x | 0.21x | 0.21x | 1.44 |
| 24* | 0.83x | 0.05x | 0.21x | 0.21x | 1.44 |
| 25 | 3.34x | 0.05x | 3.34x | 0.42x | 1.89 |
| 25* | 3.34x | 0.05x | 3.34x | 0.42x | 1.89 |
| 30 | 1.67x | 0.11x | 0.83x | 0.83x | 0.29 |
| 31 | 0.83x | 0.05x | 0.21x | 0.05x | 0.41 |
| 32 | — | 1.67x | 6.67x | 6.67x | 0.02 |
| 32* | — | 1.67x | 6.67x | 6.67x | 0.02 |
| 33 | 6.67x | 0.83x | 1.67x | 1.67x | 0.14 |
| 33* | 6.67x | 0.83x | 1.67x | 1.67x | 0.14 |
| 34 | — | 1.67x | 3.34x | 6.67x | 0.13 |
| 34* | — | 1.67x | 3.34x | 6.67x | 0.13 |
| P32 | — | 1.67x | 6.67x | 6.67x | 0.02 |
| P32* | — | 1.67x | 6.67x | 6.67x | 0.02 |
| P33 | 6.67x | 0.83x | 1.67x | 1.67x | 0.14 |
| P33* | 6.67x | 0.83x | 1.67x | 1.67x | 0.14 |
| P34 | — | 1.67x | 3.34x | 6.67x | 0.13 |
| P34* | — | 1.67x | 3.34x | 6.67x | 0.13 |
| 36 | 3.34x | 0.05 | 0.83x | 0.11x | 0.31 |
| 36* | 3.34x | 0.05 | 0.83x | 0.11x | 0.31 |
| 37 | 1.67x | 0.11x | 0.42x | 0.42x | 1.43 |
| 37* | 1.67x | 0.11x | 0.42x | 0.42x | 1.43 |
| 38 | 1.67x | 0.11x | 0.42x | 0.42x | 1.98 |
| 38* | 1.67x | 0.11x | 0.42x | 0.42x | 1.98 |
| 39 | 0.42x | 0.11x | 0.42x | 0.05x | 0.52x |

Selected examples (darobactins 9, 22, 23, 22*, 23*) were tested for their antibacterial activity in microbroth dilution assays as described above. Compounds were tested in serial dilution (0.03-64 μg/mL) and determined MICs are given in Table 12, which demonstrate superior activity of the new derivatives against Acinetobacter baumannii and Pseudomonas aeruginosa.

TABLE 12
Antibacterial activity (MIC) of pure darobactin derivatives
9, 22, 23, 22* and 23* in comparison to darobactin A

| | Darobactin derivatives; MIC [μg/mL] | | | | | |
|---|---|---|---|---|---|---|
| Bacterium | A | 9 | 22 | 22* | 23 | 23* |
| A. baumannii | 4 | 1-2 | 0.25 | 0.25 | 1-2 | 1-2 |
| DSM-30008 | | | | | | |
| A. baumannii | 16 | 2 | 2 | 2 | 4 | 4 |
| DSM-30007 | | | | | | |
| E. coli ATCC 25922 | 2 | 1-2 | 1-2 | 1-2 | 8 | 8 |
| K. pneumoniae | 2-4 | 1-2 | 2-4 | 2-4 | 4-8 | 4-8 |
| DSM-30104 | | | | | | |
| P. aeruginosa PAO1 | 0.5-1 | 0.125 | 0.5 | 0.5 | 1 | 1 |
| P. aeruginosa PA14 | 16 | 2 | 2 | 2 | 4 | 4 |
| P. aeruginosa | 4 | 1 | 1-2 | 1-2 | 2-4 | 2-4 |
| PA14ΔmexAB | | | | | | |

Activity data of exemplified darobactin derivatives 31, 32, 36 to 38, 32*, 36*-38*, 9-6F1, and 9-6F7 are summarized in Table 13 below. The compounds were tested for their antibacterial activity in microbroth dilution assays as described above. In particular, compounds were tested in serial dilution (0.03-64 μg/mL) and the determined MICs demonstrate comparable or superior microbial activity of the new derivatives.

TABLE 13
Antibacterial activity (MIC) of pure darobactin derivatives 31, 32, 36
to 38, 32*, 36*-38*, 9-6F1, and 9-6F7 in comparison to darobactin A

| | Darobactin derivatives; MIC [μg/mL] | | | | | | | | | | | |
|---|---|---|---|---|---|---|---|---|---|---|---|---|
| Bacterium | A | 31 | 32 | 36 | 37 | 38 | 32* | 36* | 37* | 38* | 9-6F1 | 9-6F7 |
| A. baumannii | 4 | 0.5-1 | 1 | 0.5 | 4-8 | 4 | 1 | 0.5 | 4-8 | 4 | 1 | 1 |
| DSM-30008 | | | | | | | | | | | | |
| A. baumannii | 16 | 2 | 1-2 | 2-4 | 16 | 8 | 1-2 | 2-4 | 16 | 8 | 2-4 | 2-4 |
| DSM-30007 | | | | | | | | | | | | |
| E. coli ATCC | 2 | 1 | 2 | 2-4 | 4-8 | 8 | 2 | 2-4 | 4-8 | 8 | 1 | 0.5 |
| 25922 | | | | | | | | | | | | |
| K. pneumoniae | 2-4 | 2-4 | 8 | 16 | 16-32 | 16-32 | 8 | 16 | 16-32 | 16-32 | 1 | 0.5-1 |
| DSM-30104 | | | | | | | | | | | | |
| P. aeruginosa | 0.5-1 | 0.25 | 2 | 0.25-0.5 | 2 | 1 | 2 | 0.25-0.5 | 2 | 1 | 1 | 0.5 |
| PAO1 | | | | | | | | | | | | |
| P. aeruginosa | 16 | 2-4 | 8 | 2-4 | 8-16 | 8-16 | 8 | 2-4 | 8-16 | 8-16 | 8 | 8 |
| PA14 DSM- | | | | | | | | | | | | |
| 19882 | | | | | | | | | | | | |
| P. aeruginosa | 4 | 1-2 | 4 | 2-4 | 8-16 | 8 | 4 | 2-4 | 8-16 | 8 | 4 | 4-8 |
| PA14ΔmexAB | | | | | | | | | | | | |

As demonstrated above, darobactin derivatives described herein have an excellent antimicrobial activity against Gram-negative bacteria, and some show selectivity against certain pathogens. Antibacterial activity of certain darobactin derivatives is superior against clinical isolates of A. baumannii, shown in Table 14 below.

TABLE 14
Antibacterial activity (MIC) of pure darobactin derivatives
9 and 22* in comparison to darobactin A and globomycin

| | Darobactin derivatives; MIC [μg/mL] | | | | |
|---|---|---|---|---|---|
| Bacterium | DA | D9 | D22 | D22* | Globomycin |
| A. baumannii 038 (OXA-23) | 16 | 4 | 0.5 | 0.5 | 32 |
| A. baumannii 045 (OXA-58) | 16-32 | 8 | 0.5 | 0.5 | 64 |
| A. baumannii 046 (OXA-40) | 8-16 | 4 | 0.5 | 0.5 | 32 |
| A. baumannii 047 (OXA-235) | 16-32 | 4-8 | 0.25 | 0.25 | 64 |
| A. baumannii 070 (NDM-1) | 8 | 2 | 0.0625 | 0.0625 | 16 |
| A. baumannii 054 (OXA-51-ISAba1) | 16-32 | 8 | 0.125 | 0.125 | >64 |

1. Compound of formula (I) or a salt thereof: 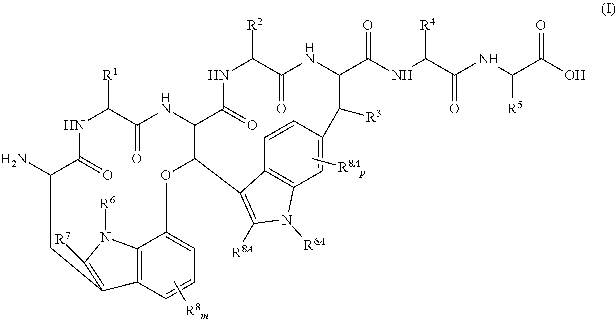
wherein
R¹, R², and R⁴ are independently selected from H, CH₃, CH₂OH, one of the following groups: 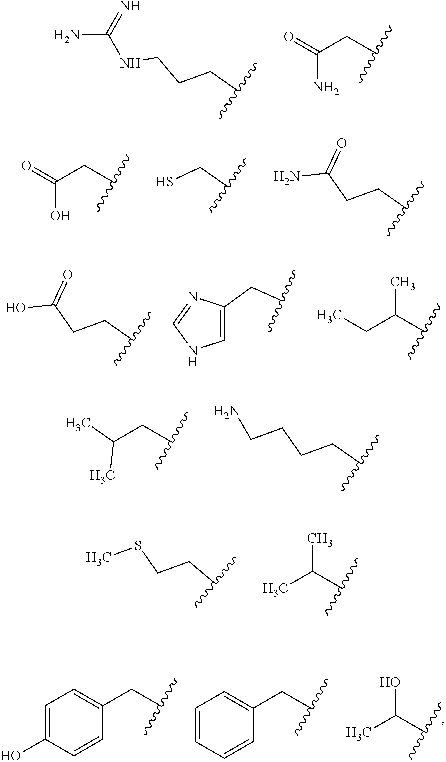
—CH₂—SR^{1A}, wherein R^{1A} is independently selected from an alkyl, an alkenyl, an alkynyl, a heteroalkyl, a cycloalkyl, a heterocycloalkyl, an alkylcycloalkyl, a heteroalkylcycloalkyl, an aryl, a heteroaryl, an aralkyl or a heteroaralkyl group, all of which groups may optionally be substituted; or —CH₂-Ind, wherein Ind is an optionally substituted indole group;
R³ is selected from H, OH, SH, COOH, CONH₂, one of the following groups: 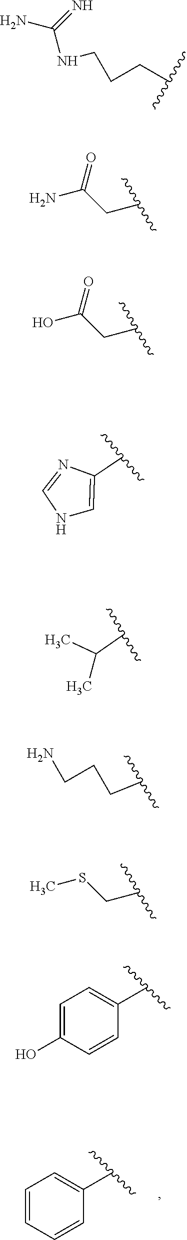
—SR^{3A}, wherein R^{3A} is selected from an alkyl, an alkenyl, an alkynyl, a heteroalkyl, a cycloalkyl, a heterocycloalkyl, an alkylcycloalkyl, a heteroalkylcycloalkyl, an aryl, a heteroaryl, an aralkyl or a heteroaralkyl group, all of which groups may optionally be substituted;
or -Ind, wherein Ind is an optionally substituted indole group;
R⁵ is a group of formula —CH₂-Ind, wherein Ind is an optionally substituted indole group
R⁶ is a hydrogen atom or a methyl group (especially a hydrogen atom);
R⁷ is a hydrogen atom, a halogen atom, a hydroxy group or a methoxy group (especially a hydrogen atom);
R⁸ is, at each occasion, independently selected from a halogen atom, a hydroxy group or a methoxy group; and
m is an integer of from 0 to 3 (especially 0 or 1; preferably 0);
R^{6A} is a hydrogen atom or a methyl group (especially a hydrogen atom);
R^{7A} is a hydrogen atom, a halogen atom, a hydroxy group or a methoxy group (especially a hydrogen atom);
R^{8A} is, at each occasion, independently selected from a halogen atom, a hydroxy group or a methoxy group; and
p is an integer of from 0 to 3 (especially 0 or 1; preferably 0).

2. The compound or a salt thereof according to claim 1, wherein the group of formula —CH₂-Ind is a group of the following formula: 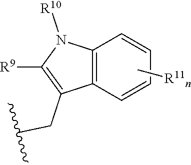
wherein R⁹ is a hydrogen atom, a halogen atom, a hydroxy group or a methoxy group (especially a hydrogen atom); R¹⁰ is a hydrogen atom or a methyl group (especially a hydrogen atom); R¹¹ is, at each occasion, independently selected from a halogen atom, a hydroxy group or a methoxy group (especially a halogen atom, preferably fluor or chlor) and n is an integer of from 0 to 4 (especially 0 or 1; preferably 0).

3. The compound or a salt thereof according to claim 1, wherein the group of formula —CH₂—SR^{1A} is:

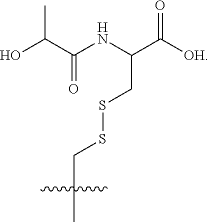 [SMILES: *CSSCC(NC(=O)C(C)O)C(=O)O]

4. The compound or a salt thereof according to claim 1, wherein R¹ is selected from the following groups: —CH₂—CONH₂, —CH₂—OH, —CH₂—CH₂—CONH₂ and —CH₂—CH(CH₃)—OH (especially wherein R¹ is a group of —CH₂—CONH₂).

5. The compound or a salt thereof according to claim 1, wherein R² is selected from the following groups: —CH₂—OH, —CH₂—CH(CH₃)—OH and —CH₃, or one of the following groups: 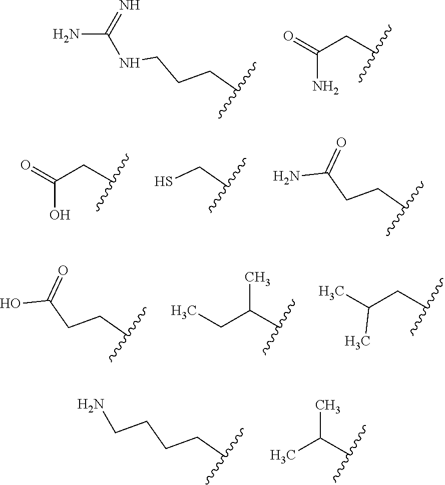
(especially wherein R² is selected from the following groups: —CH₃, —CH₂—OH, —CH₂—SH, —CH₂—CONH₂, —CH₂—CH₂—CONH₂, —CH₂—CH₂—CH₂—NH—C(═NH)—NH₂, and —CH₂—CH(CH₃)—OH).

6. The compound or a salt thereof according to claim 1, wherein R³ is a hydrogen atom or selected from the following groups: —CH₂—CH₂—CH₂—NH₂ and —CH₂—CH₂—NH—C(═NH)—NH₂ (especially wherein R³ is the group: —CH₂—CH₂—CH₂—NH₂).

7. The compound or a salt thereof according to claim 1, wherein R⁴ is selected from the following groups: —CH₂—OH, —CH₃ and —CH₂—CH₂—CH₂—NH—C(═NH)—NH₂; one of the following groups: 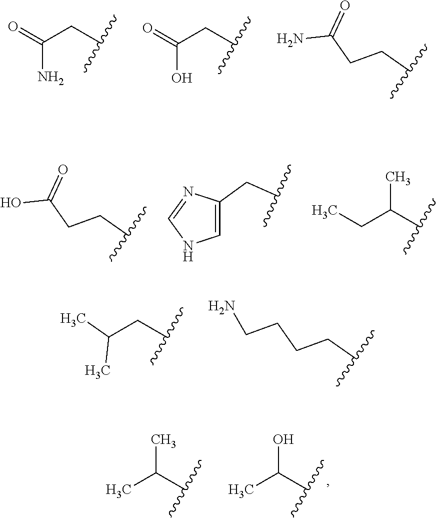
or
wherein R⁴ is a group of formula —CH₂—SR^{4A}, wherein R^{4A} is selected from a hydrogen atom, an alkyl, an alkenyl, an alkynyl, a heteroalkyl, a cycloalkyl, a heterocycloalkyl, an alkylcycloalkyl, a heteroalkylcycloalkyl, an aryl, a heteroaryl, an aralkyl or a heteroaralkyl group, all of which groups may optionally be substituted (especially wherein R⁴ is selected from the following groups: CH₃, CH₂OH, 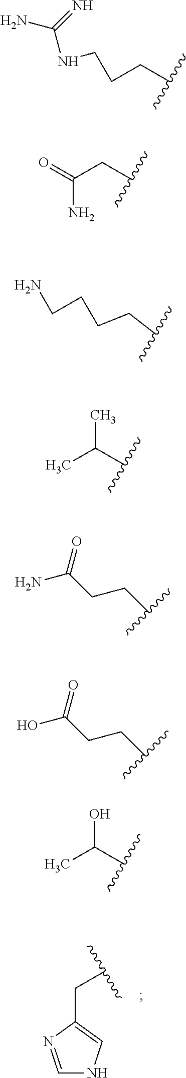
preferably R⁴ is selected from —CH₂—OH, —CH₃ and —CH₂—CH₂—CH₂—NH—C(═NH)—NH₂).

8. The compound or a salt thereof according to claim 1, wherein R⁵ is a group of the following formula: 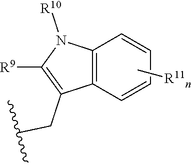
wherein R⁹ is a hydrogen atom; R¹⁰ is a hydrogen atom; R¹¹ is, at each occasion, independently selected from a halogen atom, a hydroxy group or a methoxy group (especially a halogen atom, preferably fluor or chlor) and n is an integer of from 0 to 4 (especially 0 or 1; preferably 0).

9. The compound or a salt thereof according to claim 1, wherein each of R⁶, R^{6A}, R⁷, and R^{7A} is a hydrogen atom.

10. The compound or a salt thereof according to claim 1, wherein R⁸ and/or R^{8A} is a halogen atom (especially a fluor or chlor atom, preferably a fluor atom).

11. The compound or a salt thereof according to claim 1, wherein the compound has structural formula (Ia): 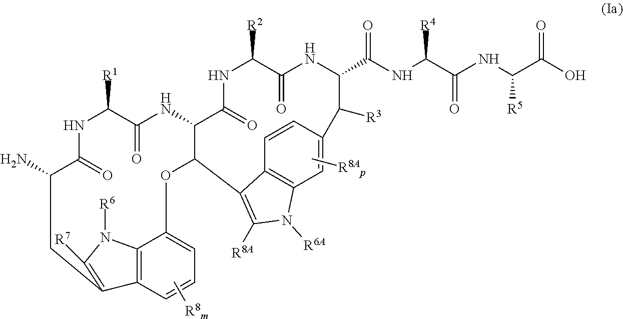
wherein R¹, R², R³, R⁴, R⁵, R⁶, R⁷, R⁸, R^{6A}, R^{7A}, R^{8A}, m and p are as defined in any one of the preceding claims.

12. Pharmaceutical composition comprising a compound or a salt thereof according to claim 1 and optionally i) one or more carrier substances and/or ii) one or more adjuvants and/or iii) one or more further active pharmaceutical ingredient(s).

13. (canceled)

14. A method of producing a compound according to claim 1, wherein the compound is a compound of formula (II): 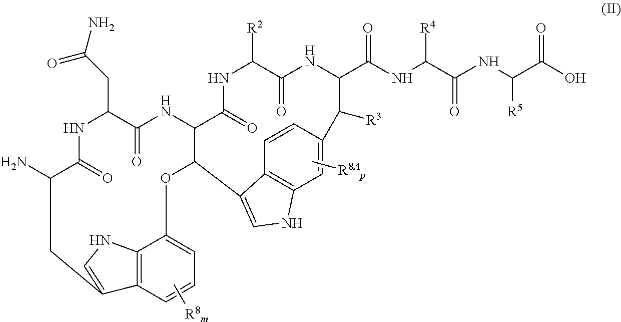
wherein
R² and R⁴ each independently represents H, CH₃, CH₂OH or a group: 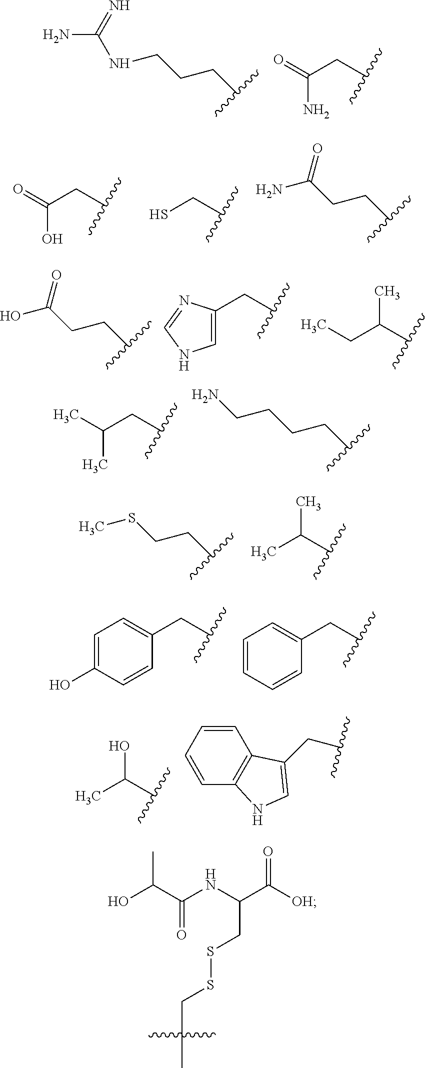
R³ is a group: 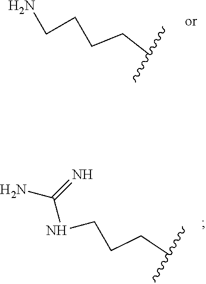 [SMILES: *CCCCN ; *CCCNC(=N)N]
R⁵ is a group of the following formula: 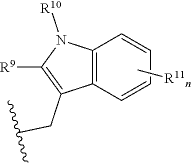
wherein R⁹ is a hydrogen or halogen atom (especially a hydrogen atom); R¹⁰ is a hydrogen atom or a methyl group (especially a hydrogen atom); R¹¹ is, at each occasion, independently selected from a halogen atom (especially fluor or chlor) and n is an integer of from 0 to 4 (especially 0 or 1; preferably 0)
and
wherein the method comprises the steps of:
(a) providing a recombinant host capable of producing said compound of formula (II) (or (IIa), (IIa′) or (IIa″)), wherein said recombinant host harbors at least one synthetic or recombinant nucleic acid encoding a biosynthetic gene cluster (BGC) of the compound, which BGC has:
(i) a sequence identity to the full-length sequence of SEQ ID NO. 86 of at least 95%, 96%, 97%, 98%, 98.5%, 99%, or 99.5% to 100%; or
(ii) a sequence completely complementary to any nucleic acid sequence of (i);
(b) cultivating said recombinant host for a time sufficient for said recombinant host to produce the compound of formula (II) (or (IIa), (IIa′) or (IIa″));
(c) isolating the compound from said recombinant host or from cultivation supernatant, thereby producing the compound of formula (II) (or (IIa), (IIa′) or (IIa″)).

15. A recombinant host comprising a heterologous nucleic acid encoding a compound of formula (II) as defined in claim 14.

16. The recombinant host of claim 15, wherein the recombinant host is a microorganism, preferably a bacterium or yeast, such as a bacterial cell of the species E. coli, such as E. coli BL21(DE3) (especially DSM 33798 and/or DSM 33799); Corynebacteria, such as C. glutamicum; Bacillus; Lactobacillus, such as Lactococcus lactis; or Streptomyces, such as S. albus and S. lividans; or a yeast cell of the species Saccharomyces cerevisiae, Saccharomyces pombe, Pichia pastoris or Yarrowia lipolytica.

17. A vector comprising at least one nucleic acid as defined in claim 14, such as a vector capable of autonomous replication in bacteria or yeast, preferably an isolated and purified plasmid capable of autonomous replication in bacteria; preferably capable of autonomous replication in bacteria of the species Lactobacillus, such as Lactococcus actis, or E. coli, more preferably capable of autonomous replication in bacteria of the species E. coli, especially DSM 33802 and/or DSM 33803.

18.-19. (canceled)

20. A method of treating a subject suffering from or susceptible to a bacterial infection comprising:
administering to the subject an effective amount of compound of claim 1.

21. The method of claim 20 wherein the bacterial infection is caused by Gram-negative bacteria.

22. The method of claim 20 wherein the subject is identified as suffering from or susceptible to a bacterial infection, and the compound is administered to the identified subject.

23. The method of claim 21 wherein the subject is identified as suffering from or susceptible to a bacterial infection, and the compound is administered to the identified subject
